(19) United States
(12) Patent Application Publication
(10) Pub. No.: US 2023/0180599 A1
(43) Pub. Date: Jun. 8, 2023

(54) LIGHT-EMITTING DEVICE

(71) Applicant: Samsung Display Co., Ltd., Yongin-si (KR)

(72) Inventors: Jeongmin Lee, Yongin-si (KR); Minji Kim, Yongin-si (KR); Hyunbin Park, Yongin-si (KR); Eunjae Jeong, Yongin-si (KR); Jiyong Choi, Yongin-si (KR); Sanghyun Han, Yongin-si (KR)

(21) Appl. No.: 18/060,915

(22) Filed: Dec. 1, 2022

(30) Foreign Application Priority Data
Dec. 6, 2021 (KR) 10-2021-0173062

(51) Int. Cl. H01L 51/00; C09K 11/06; C07C 211/61; C07D 307/91

(52) U.S. Cl. CPC H01L 51/006; C07C 211/61; C07D 307/91; C09K 11/06; H01L 51/0061; C07C 2602/42; C07C 2603/18; C07C 2603/97; C09K 2211/1007; C09K 2211/1011; C09K 2211/1014; C09K 2211/1018; H01L 27/3244; H01L 51/0056; H01L 51/0058; H01L 51/0073; H01L 51/5056

(57) ABSTRACT

Provided is a light-emitting device including a first electrode; a second electrode facing the first electrode; and an interlayer between the first electrode and the second electrode and including a layer including a compound represented by Formula 1.

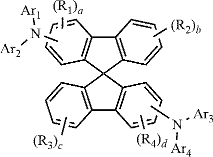

### CROSS-REFERENCE TO RELATED APPLICATION

This application claims priority to and the benefit of Korean Patent Application No. 10-2021-0173062, filed on Dec. 6, 2021, in the Korean Intellectual Property Office, the entire content of which is hereby incorporated by reference.

### BACKGROUND

### 1. Field

One or more embodiments of the present disclosure relate to a light-emitting device.

### 2. Description of the Related Art

Organic light-emitting devices are self-emissive devices that have wide viewing angles, high contrast ratios, short response times, and excellent characteristics in terms of luminance, driving voltage, and response speed, compared to other devices in the art.

Organic light-emitting devices may include a first electrode on a substrate, and a hole transport region, an emission layer, an electron transport region, and a second electrode sequentially stacked on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. These excitons transition from an excited state to a ground state, thereby generating light.

### SUMMARY

One or more embodiments of the present disclosure include a light-emitting device having improved efficiency and lifespan.

Additional aspects of embodiments will be set forth in part in the description which follows and, in part, will be apparent from the description, or may be learned by practice of the presented embodiments of the disclosure.

According to one or more embodiments, provided is a light-emitting device including:
a first electrode,
a second electrode facing the first electrode, and
an interlayer between the first electrode and the second electrode and including an emission layer,
wherein the interlayer includes a layer including a compound represented by Formula 1.

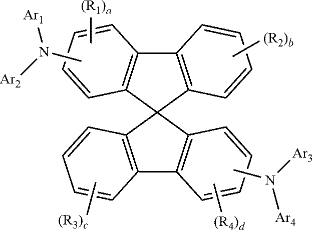

In Formula 1,
Ar₁ to Ar₄ and R₁ to R₄ may each independently be selected from hydrogen, deuterium, —F, —Cl., —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₈-C₆₀ monovalent non-aromatic condensed polycyclic group unsubstituted or substituted with at least one R₁₀ₐ, a C₁-C₆₀ monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), and —P(═S)(Q₁)(Q₂),
a and d may each independently be an integer from 1 to 3,
b and c may each independently be an integer from 1 to 4,
the sum of a and d may be an integer of 1 or more, and a case in which each of R₁ and R₄ is hydrogen or deuterium may be excluded,
R₁₀ₐ may be:
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, -Si(Q₁₁)(Q₁₂)(Q₁₃), -N(Q₁₁)(Q₁₂), -B(Q₁₁)(Q₁₂), —C(═O)(Q₁₁), —S(═O)₂(Q₁₁), —P(═O)(O₁₁)(O₁₂), or any combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, -Si(Q₂₁)(Q₂₂)(Q₂₃), -N(Q₂₁)(Q₂₂), -B(Q₂₁)(Q₂₂), —C(═O)(Q₂₁), —S(═O)₂(Q₂₁), —P(═O)(Q₂₁)(Q₂₂), or any combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments,

provided is an electronic apparatus including the light-emitting device.

According to one or more embodiments,

provided is the compound represented by Formula 1.

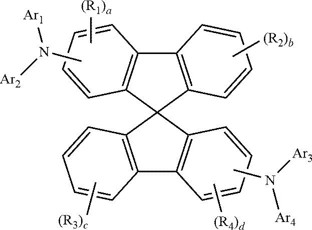

In Formula 1, substituents and variables are respectively the same as those described above.

### BRIEF DESCRIPTION OF THE DRAWINGS

The above and other aspects and features of certain embodiments of the present disclosure will be more apparent from the following description taken in conjunction with the accompanying drawings, in which:

FIG. 1 is a schematic view of a light-emitting device according to an embodiment;

FIG. 2 is a cross-sectional view of an electronic apparatus according to an embodiment; and

FIG. 3 is a cross-sectional view of an electronic apparatus according to another embodiment.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of embodiments of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

According to one or more embodiments, a light-emitting device may include:
a first electrode;
a second electrode facing the first electrode; and
an interlayer between the first electrode and the second electrode and including an emission layer,
wherein the interlayer may include a layer including a compound represented by Formula 1:

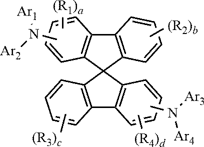

wherein, in Formula 1,
Ar₁ to Ar₄ and R₁ to R₄ may each independently be selected from hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₈-C₆₀ monovalent non-aromatic condensed polycyclic group unsubstituted or substituted with at least one R₁₀ₐ, a C₁-C₆₀ monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), and —P(═S)(Q₁)(Q₂),
a and d may each independently be an integer from 1 to 3,
b and c may each independently be an integer from 1 to 4,
the sum of a and d may be an integer of 1 or more, and a case in which each of R₁ and R₄ is hydrogen or deuterium may be excluded,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, -Si(Q₁₁)(Q₁₂)(Q₁₃), -N(Q₁₁)(Q₁₂), -B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, -Si(Q₂₁)(Q₂₂)(Q₂₃), -N(Q₂₁)(Q₂₂), -B(Q₂₁)(Q₂₂), —C(═O)(Q₂₁), —S(═O)₂(Q₂₁), —P(═O)(Q₂₁)(Q₂₂), or any combination thereof; or
-Si(Q₃₁)(Q₃₂)(Q₃₃), -N(Q₃₁)(Q₃₂), -B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, in the light-emitting device, the first electrode may be an anode, and the second electrode may be a cathode, and

the interlayer may further include an electron transport region between the second electrode and the emission layer and including a hole blocking layer, an electron injection layer, an electron transport layer, or any combination thereof.

In an embodiment, in the light-emitting device, the first electrode may be an anode, and the second electrode may be a cathode, and

the interlayer may further include a hole transport region between the first electrode and the emission layer and including a hole transport layer; and an electron blocking layer, a hole injection layer, or any combination thereof.

In an embodiment, in Formula 1, moiety —NAr₁Ar₂ and R₁ may be adjacent to each other.

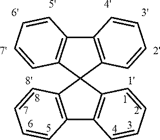 [SMILES: c1ccc2c(c1)-c1ccccc1C21c2ccccc2-c2ccccc21]

A core of Formula 1 having numbered carbon atom positions is shown above for reference. In the above core of Formula 1, for example, moiety —NAr₁Ar₂ may be present at position 8′, and R₁ may be present at position 7′. For example, moiety —NAr₁Ar₂ may be present at position 7′, and R₁ may be present at position 6′. For example, moiety —NAr₁Ar₂ may be present at position 6′, and R₁ may be present at position 5′. For example, moiety —NAr₁Ar₂ may be present at position 5′, and R₁ may be present at position 6′.

In an embodiment, in Formula 1, moiety —NAr₃Ar₄ and R₄ may be adjacent to each other.

In the above core of Formula 1, for example, moiety —NAr₃Ar₄ may be present at position 1, and R₄ may be present at position 2. For example, moiety —NAr₃Ar₄ may be present at position 2, and R₄ may be present at position 3. For example, moiety —NAr₃Ar₄ may be present at position 3, and R₄ may be present at position 4. For example, moiety —NAr₃Ar₄ may be present at position 4, and R₄ may be present at position 3.

In Formula 1, the sum of a and d may be an integer of 1 or more, and a case in which each of R₁ and R₄ is hydrogen or deuterium may be excluded. For example, the sum of a and d may be an integer from 1 to 4.

For example, in the core of Formula 1, R₁ adjacent to moiety —NAr₁Ar₂ may be present, and/or R₄ adjacent to moiety —NAr₃Ar₄ may be present.

Due to the presence of a substituent adjacent to moiety —NAr₁Ar₂ and/or moiety —NAr₃Ar₄ in the core, the light-emitting device including the compound represented by Formula 1 of the present disclosure may exhibit improved efficiency and lifespan.

In an embodiment, Formula 1 may be represented by one selected from Formulae 2-1 to 2-4:

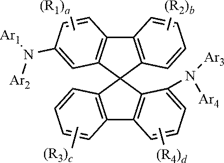

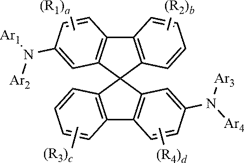

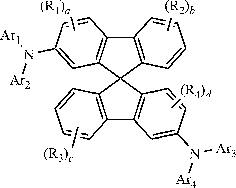

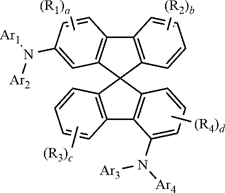

wherein, in Formulae 2-1 to 2-4, substituents and variables are respectively the same as those described with respect to Formula 1.

In an embodiment, Formula 1 may be represented by one of Formulae 3-1 or 3-2:

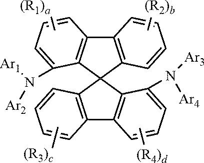

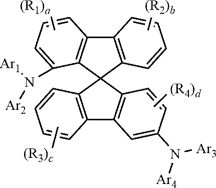

wherein, in Formulae 3-1 and 3-2, substituents and variables are respectively the same as those described with respect to Formula 1.

In an embodiment, Formula 1 may be represented by one of Formulae 4-1 or 4-2:

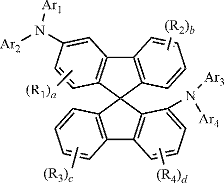

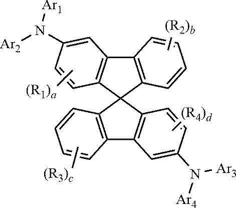

wherein, in Formulae 4-1 and 4-2, substituents and variables are respectively the same as those described with respect to Formula 1.

In an embodiment, Formula 1 may be represented by one selected from Formulae 5-1 to 5-3:

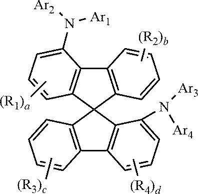

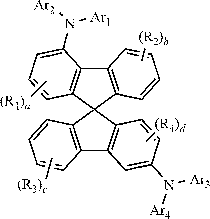

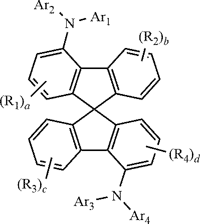

wherein, in Formulae 5-1 to 5-3, substituents and variables are respectively the same as those described with respect to in Formula 1.

In an embodiment, Ar₁ to Ar₄ may each independently be selected from groups represented by Formulae 6-1 to 6-3:

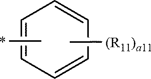

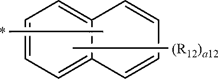

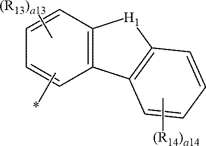

wherein, in Formulae 6-1 to 6-3, H₁ may represent O, S, NR₂₁, or CR₂₂R₂₃, and R₁₁ to R₁₄ and R₂₁ to R₂₃ may each independently be selected from hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), and —P(═O)(Q₁)(Q₂),
a11 may be an integer from 1 to 5,
a12 may be an integer from 1 to 7,
a13 may be an integer from 1 to 3,
a14 may be an integer from 1 to 4, and
Q₁ to Q₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, R₁ to R₄ may each independently be selected from a C₁-C₆₀ alkyl group and groups represented by Formulae 7-1 to 7-4:

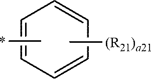

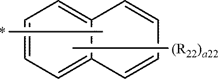

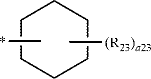

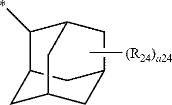

wherein, in Formulae 7-1 to 7-4, R₂₁ to R₂₄ may each independently be selected from hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), and —P(═O)(Q₁)(Q₂),
a21 and a23 may each independently be an integer from 1 to 5,
a22 may be an integer from 1 to 7,
a24 may be an integer from 1 to 9, and
Q₁ to Q₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In Formula 7-4, R₂₄ may be present at any position other than adamantane’s binding site to the core.

In an embodiment, the compound represented by Formula 1 may be selected from the following compounds:

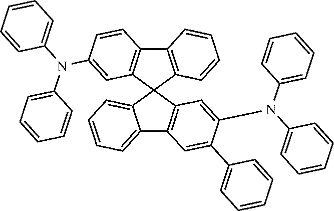 [SMILES: c1ccc(-c2cc3c(cc2N(c2ccccc2)c2ccccc2)C2(c4ccccc4-c4ccc(N(c5ccccc5)c5ccccc5)cc42)c2ccccc2-3)cc1]

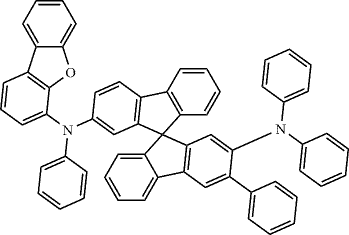 [SMILES: c1ccc(-c2cc3c(cc2N(c2ccccc2)c2ccccc2)C2(c4ccccc4-c4ccc(N(c5ccccc5)c5cccc6c5oc5ccccc56)cc42)c2ccccc2-3)cc1]

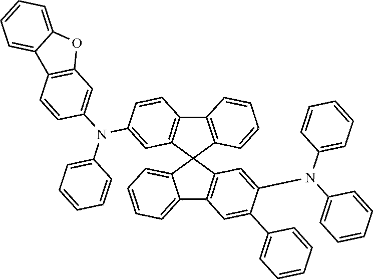 [SMILES: c1ccc(-c2cc3c(cc2N(c2ccccc2)c2ccccc2)C2(c4ccccc4-c4ccc(N(c5ccccc5)c5ccc6c(c5)oc5ccccc56)cc42)c2ccccc2-3)cc1]

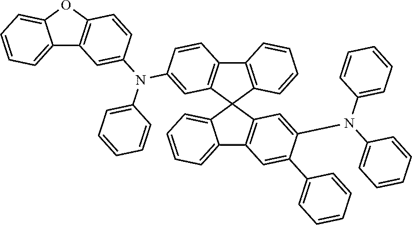 [SMILES: c1ccc(-c2cc3c(cc2N(c2ccccc2)c2ccccc2)C2(c4ccccc4-c4ccc(N(c5ccccc5)c5ccc6oc7ccccc7c6c5)cc42)c2ccccc2-3)cc1]

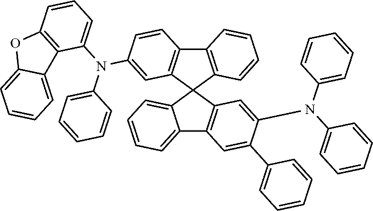 [SMILES: c1ccc(-c2cc3c(cc2N(c2ccccc2)c2ccccc2)C2(c4ccccc4-c4ccc(N(c5ccccc5)c5cccc6oc7ccccc7c56)cc42)c2ccccc2-3)cc1]

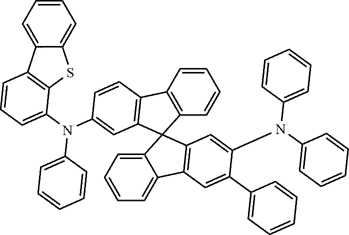 [SMILES: c1ccc(-c2cc3c(cc2N(c2ccccc2)c2ccccc2)C2(c4ccccc4-c4ccc(N(c5ccccc5)c5cccc6c5sc5ccccc56)cc42)c2ccccc2-3)cc1]

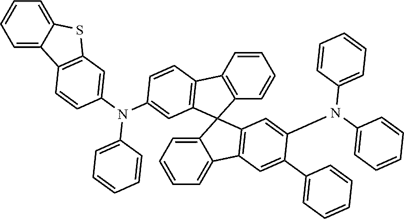 [SMILES: c1ccc(-c2cc3c(cc2N(c2ccccc2)c2ccccc2)C2(c4ccccc4-c4ccc(N(c5ccccc5)c5ccc6c(c5)sc5ccccc56)cc42)c2ccccc2-3)cc1]

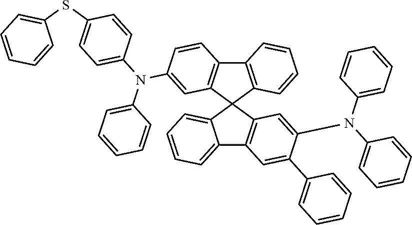 [SMILES: c1ccc(Sc2ccc(N(c3ccccc3)c3ccc4c(c3)C3(c5ccccc5-4)c4ccccc4-c4cc(-c5ccccc5)c(N(c5ccccc5)c5ccccc5)cc43)cc2)cc1]

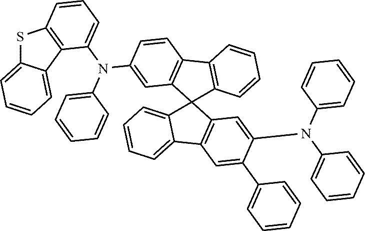 [SMILES: c1ccc(-c2cc3c(cc2N(c2ccccc2)c2ccccc2)C2(c4ccccc4-c4ccc(N(c5ccccc5)c5cccc6sc7ccccc7c56)cc42)c2ccccc2-3)cc1]

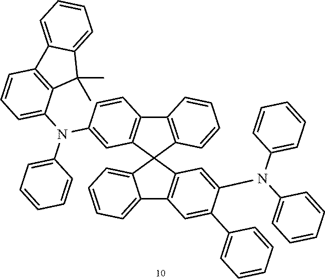

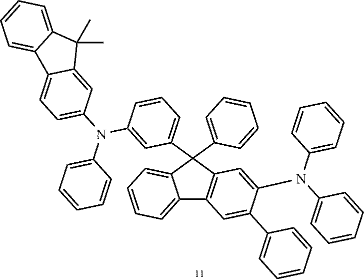

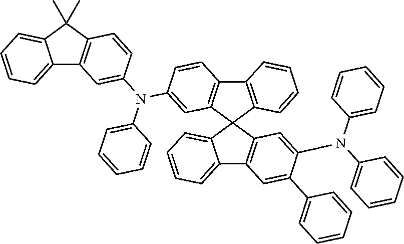 [SMILES: CC1(C)c2ccccc2-c2cc(N(c3ccccc3)c3ccc4c(c3)C3(c5ccccc5-4)c4ccccc4-c4cc(-c5ccccc5)c(N(c5ccccc5)c5ccccc5)cc43)ccc21]

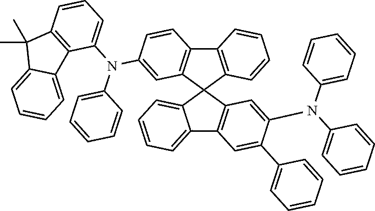 [SMILES: CC1(C)c2ccccc2-c2c(N(c3ccccc3)c3ccc4c(c3)C3(c5ccccc5-4)c4ccccc4-c4cc(-c5ccccc5)c(N(c5ccccc5)c5ccccc5)cc43)cccc21]

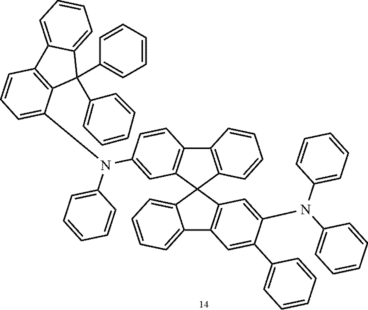

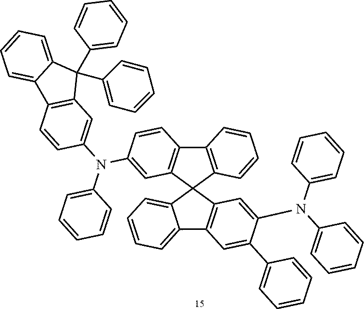

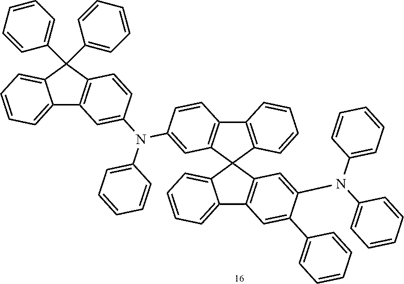

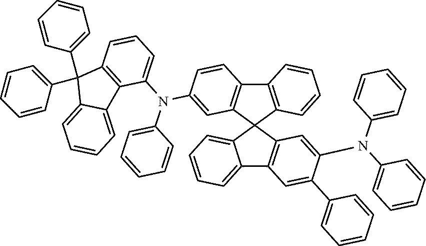 [SMILES: c1ccc(-c2cc3c(cc2N(c2ccccc2)c2ccccc2)C2(c4ccccc4-c4ccc(N(c5ccccc5)c5cccc6c5-c5ccccc5C6(c5ccccc5)c5ccccc5)cc42)c2ccccc2-3)cc1]

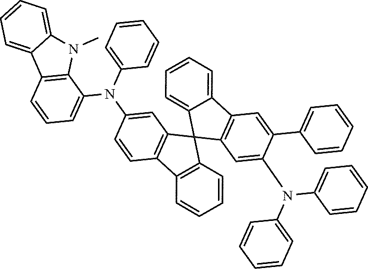 [SMILES: Cn1c2ccccc2c2cccc(N(c3ccccc3)c3ccc4c(c3)C3(c5ccccc5-4)c4ccccc4-c4cc(-c5ccccc5)c(N(c5ccccc5)c5ccccc5)cc43)c21]

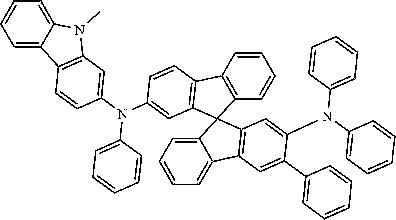 [SMILES: Cn1c2ccccc2c2ccc(N(c3ccccc3)c3ccc4c(c3)C3(c5ccccc5-4)c4ccccc4-c4cc(-c5ccccc5)c(N(c5ccccc5)c5ccccc5)cc43)cc21]

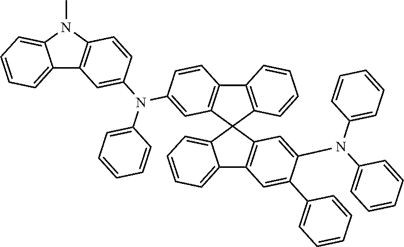 [SMILES: Cn1c2ccccc2c2cc(N(c3ccccc3)c3ccc4c(c3)C3(c5ccccc5-4)c4ccccc4-c4cc(-c5ccccc5)c(N(c5ccccc5)c5ccccc5)cc43)ccc21]

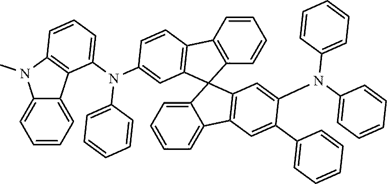 [SMILES: Cn1c2ccccc2c2c(N(c3ccccc3)c3ccc4c(c3)C3(c5ccccc5-4)c4ccccc4-c4cc(-c5ccccc5)c(N(c5ccccc5)c5ccccc5)cc43)cccc21]

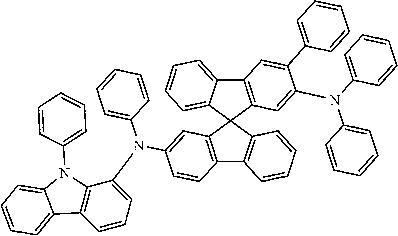 [SMILES: c1ccc(-c2cc3c(cc2N(c2ccccc2)c2ccccc2)C2(c4ccccc4-c4ccc(N(c5ccccc5)c5cccc6c7ccccc7n(-c7ccccc7)c56)cc42)c2ccccc2-3)cc1]

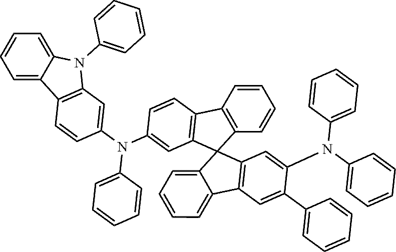 [SMILES: c1ccc(-c2cc3c(cc2N(c2ccccc2)c2ccccc2)C2(c4ccccc4-c4ccc(N(c5ccccc5)c5ccc6c7ccccc7n(-c7ccccc7)c6c5)cc42)c2ccccc2-3)cc1]

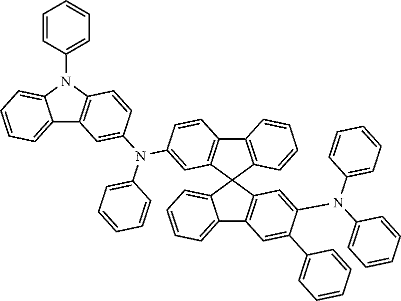 [SMILES: c1ccc(-c2cc3c(cc2N(c2ccccc2)c2ccccc2)C2(c4ccccc4-c4ccc(N(c5ccccc5)c5ccc6c(c5)c5ccccc5n6-c5ccccc5)cc42)c2ccccc2-3)cc1]

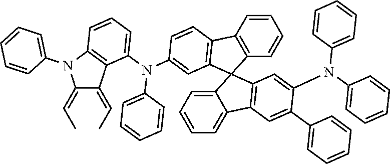 [SMILES: C/C=c1\c(=C/C)n(-c2ccccc2)c2cccc(N(c3ccccc3)c3ccc4c(c3)C3(c5ccccc5-4)c4ccccc4-c4cc(-c5ccccc5)c(N(c5ccccc5)c5ccccc5)cc43)c12]

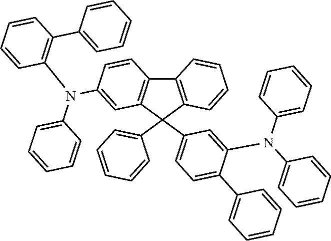 [SMILES: c1ccc(-c2ccccc2N(c2ccccc2)c2ccc3c(c2)C(c2ccccc2)(c2ccc(-c4ccccc4)c(N(c4ccccc4)c4ccccc4)c2)c2ccccc2-3)cc1]

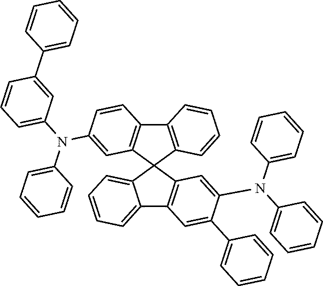 [SMILES: c1ccc(-c2cccc(N(c3ccccc3)c3ccc4c(c3)C3(c5ccccc5-4)c4ccccc4-c4cc(-c5ccccc5)c(N(c5ccccc5)c5ccccc5)cc43)c2)cc1]

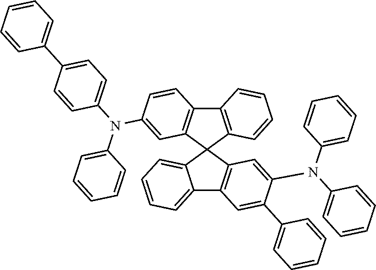 [SMILES: c1ccc(-c2ccc(N(c3ccccc3)c3ccc4c(c3)C3(c5ccccc5-4)c4ccccc4-c4cc(-c5ccccc5)c(N(c5ccccc5)c5ccccc5)cc43)cc2)cc1]

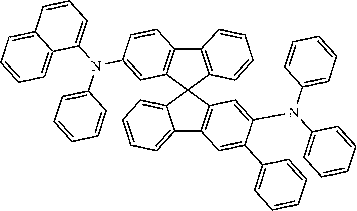 [SMILES: c1ccc(-c2cc3c(cc2N(c2ccccc2)c2ccccc2)C2(c4ccccc4-c4ccc(N(c5ccccc5)c5cccc6ccccc56)cc42)c2ccccc2-3)cc1]

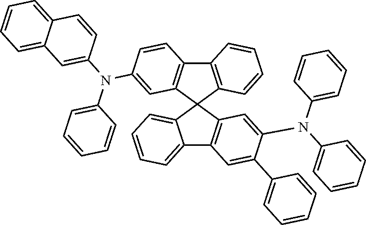 [SMILES: c1ccc(-c2cc3c(cc2N(c2ccccc2)c2ccccc2)C2(c4ccccc4-c4ccc(N(c5ccccc5)c5ccc6ccccc6c5)cc42)c2ccccc2-3)cc1]

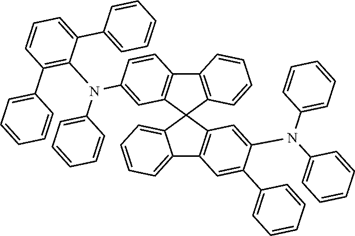 [SMILES: c1ccc(-c2cc3c(cc2N(c2ccccc2)c2ccccc2)C2(c4ccccc4-c4ccc(N(c5ccccc5)c5c(-c6ccccc6)cccc5-c5ccccc5)cc42)c2ccccc2-3)cc1]

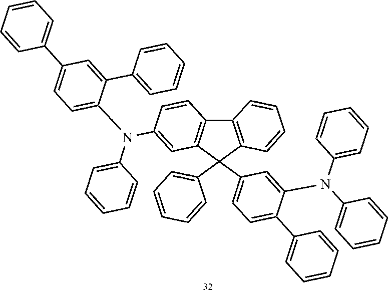

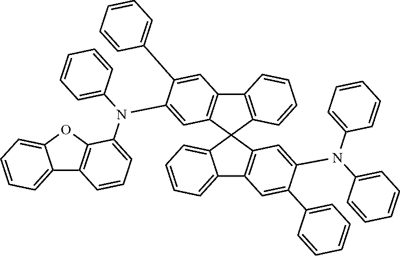 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4cccc5c4oc4ccccc45)cc32)cc1]

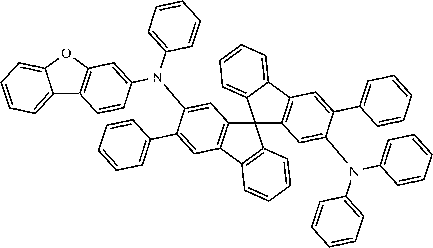 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5c(c4)oc4ccccc45)cc32)cc1]

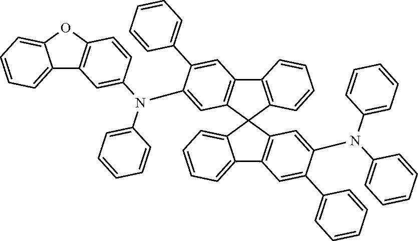 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5oc6ccccc6c5c4)cc32)cc1]

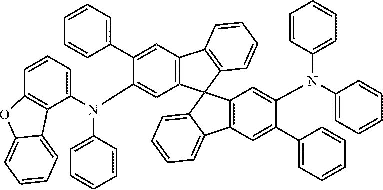 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4cccc5oc6ccccc6c45)cc32)cc1]

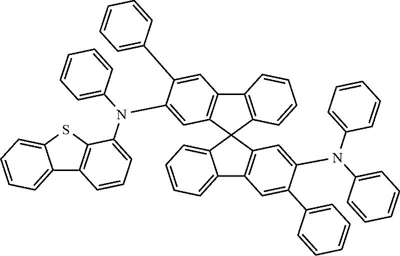 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4cccc5c4sc4ccccc45)cc32)cc1]

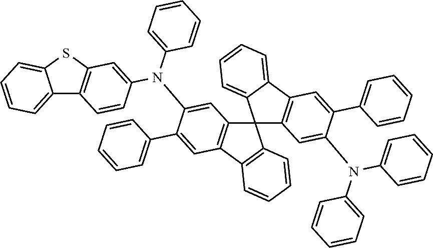 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5c(c4)sc4ccccc45)cc32)cc1]

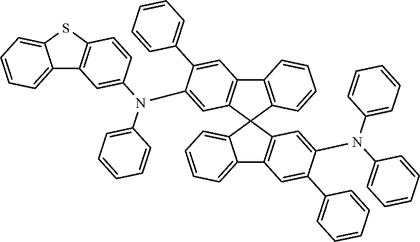 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5sc6ccccc6c5c4)cc32)cc1]

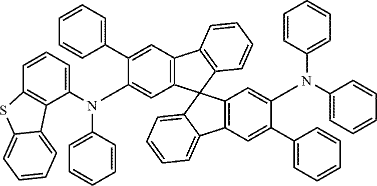 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4cccc5sc6ccccc6c45)cc32)cc1]

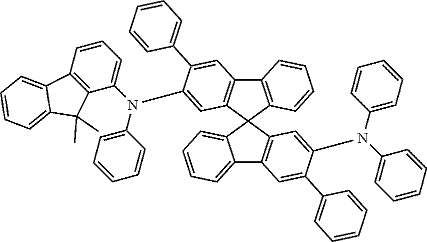 [SMILES: CC1(C)c2ccccc2-c2cccc(N(c3ccccc3)c3cc4c(cc3-c3ccccc3)-c3ccccc3C43c4ccccc4-c4cc(-c5ccccc5)c(N(c5ccccc5)c5ccccc5)cc43)c21]

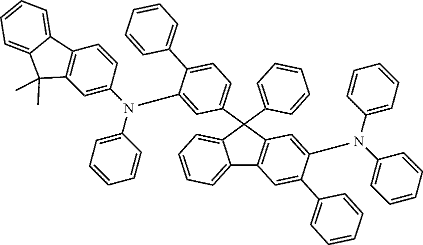 [SMILES: CC1(C)c2ccccc2-c2ccc(N(c3ccccc3)c3cc(C4(c5ccccc5)c5ccccc5-c5cc(-c6ccccc6)c(N(c6ccccc6)c6ccccc6)cc54)ccc3-c3ccccc3)cc21]

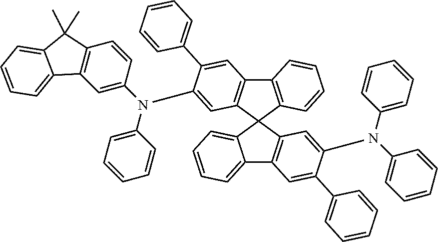 [SMILES: CC1(C)c2ccccc2-c2cc(N(c3ccccc3)c3cc4c(cc3-c3ccccc3)-c3ccccc3C43c4ccccc4-c4cc(-c5ccccc5)c(N(c5ccccc5)c5ccccc5)cc43)ccc21]

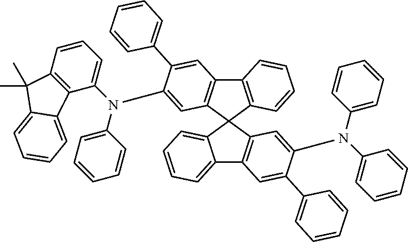 [SMILES: CC1(C)c2ccccc2-c2c(N(c3ccccc3)c3cc4c(cc3-c3ccccc3)-c3ccccc3C43c4ccccc4-c4cc(-c5ccccc5)c(N(c5ccccc5)c5ccccc5)cc43)cccc21]

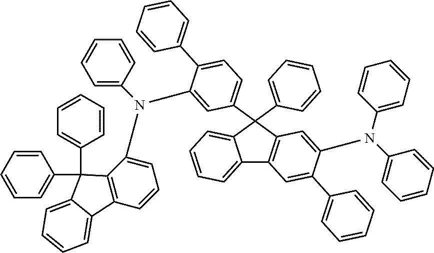 [SMILES: c1ccc(-c2cc3c(cc2N(c2ccccc2)c2ccccc2)C(c2ccccc2)(c2ccc(-c4ccccc4)c(N(c4ccccc4)c4cccc5c4C(c4ccccc4)(c4ccccc4)c4ccccc4-5)c2)c2ccccc2-3)cc1]

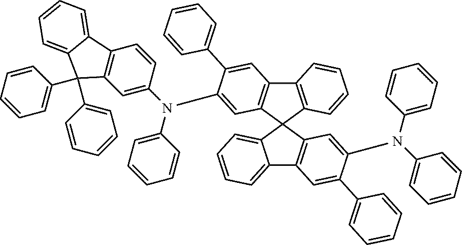 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5c(c4)C(c4ccccc4)(c4ccccc4)c4ccccc4-5)cc32)cc1]

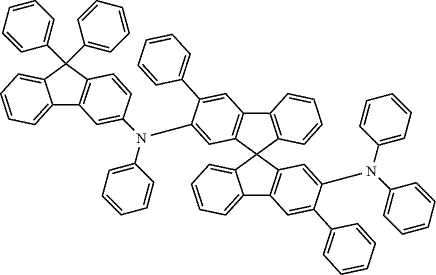 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5c(c4)-c4ccccc4C5(c4ccccc4)c4ccccc4)cc32)cc1]

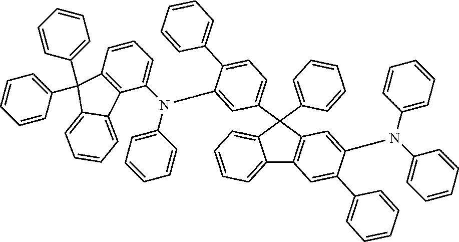 [SMILES: c1ccc(-c2cc3c(cc2N(c2ccccc2)c2ccccc2)C(c2ccccc2)(c2ccc(-c4ccccc4)c(N(c4ccccc4)c4cccc5c4-c4ccccc4C5(c4ccccc4)c4ccccc4)c2)c2ccccc2-3)cc1]

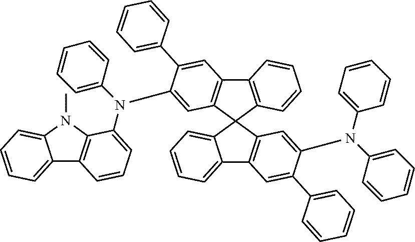 [SMILES: Cn1c2ccccc2c2cccc(N(c3ccccc3)c3cc4c(cc3-c3ccccc3)-c3ccccc3C43c4ccccc4-c4cc(-c5ccccc5)c(N(c5ccccc5)c5ccccc5)cc43)c21]

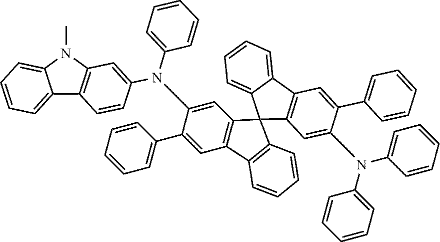 [SMILES: Cn1c2ccccc2c2ccc(N(c3ccccc3)c3cc4c(cc3-c3ccccc3)-c3ccccc3C43c4ccccc4-c4cc(-c5ccccc5)c(N(c5ccccc5)c5ccccc5)cc43)cc21]

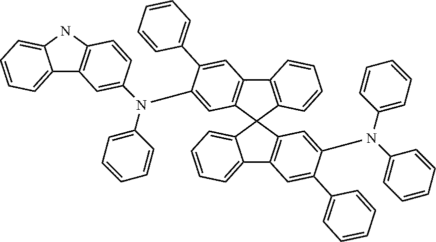 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5[nH]c6ccccc6c5c4)cc32)cc1]

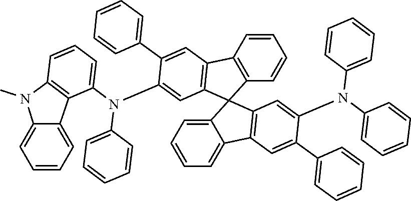 [SMILES: Cn1c2ccccc2c2c(N(c3ccccc3)c3cc4c(cc3-c3ccccc3)-c3ccccc3C43c4ccccc4-c4cc(-c5ccccc5)c(N(c5ccccc5)c5ccccc5)cc43)cccc21]

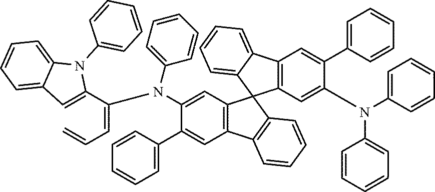 [SMILES: C=C/C=C(\c1cc2ccccc2n1-c1ccccc1)N(c1ccccc1)c1cc2c(cc1-c1ccccc1)-c1ccccc1C21c2ccccc2-c2cc(-c3ccccc3)c(N(c3ccccc3)c3ccccc3)cc21]

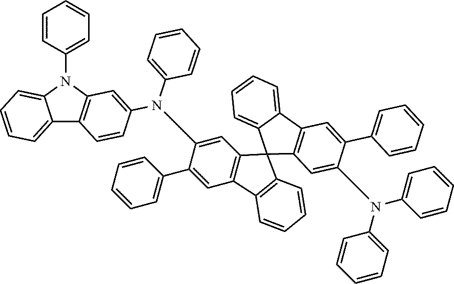 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5c6ccccc6n(-c6ccccc6)c5c4)cc32)cc1]

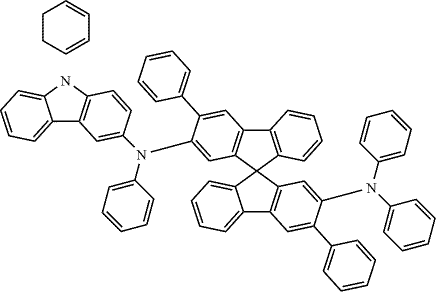

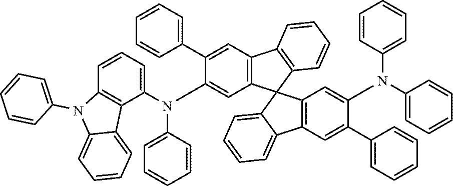 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4cccc5c4c4ccccc4n5-c4ccccc4)cc32)cc1]

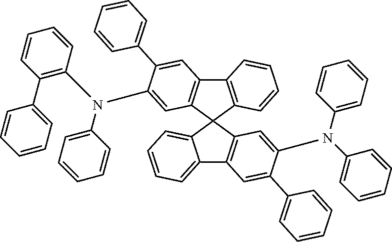 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccccc4-c4ccccc4)cc32)cc1]

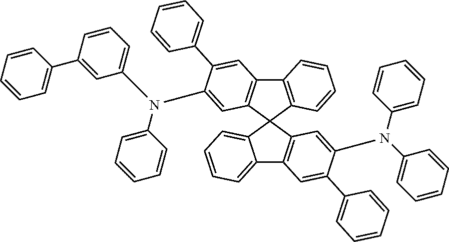 [SMILES: c1ccc(-c2cccc(N(c3ccccc3)c3cc4c(cc3-c3ccccc3)-c3ccccc3C43c4ccccc4-c4cc(-c5ccccc5)c(N(c5ccccc5)c5ccccc5)cc43)c2)cc1]

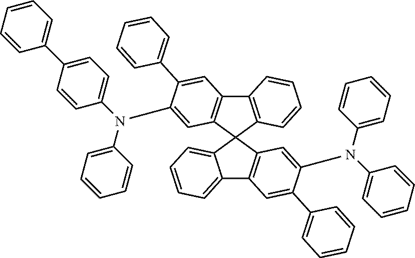 [SMILES: c1ccc(-c2ccc(N(c3ccccc3)c3cc4c(cc3-c3ccccc3)-c3ccccc3C43c4ccccc4-c4cc(-c5ccccc5)c(N(c5ccccc5)c5ccccc5)cc43)cc2)cc1]

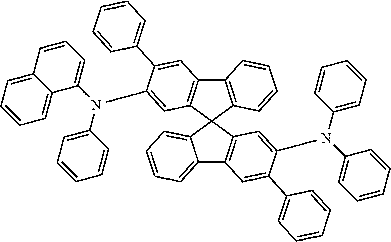 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4cccc5ccccc45)cc32)cc1]

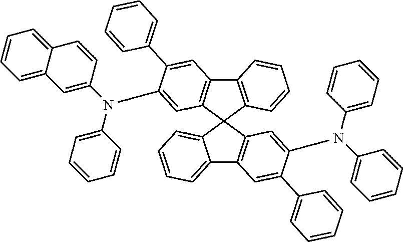 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5ccccc5c4)cc32)cc1]

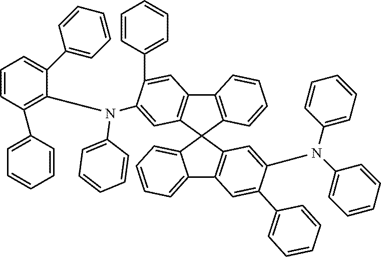 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4c(-c5ccccc5)cccc4-c4ccccc4)cc32)cc1]

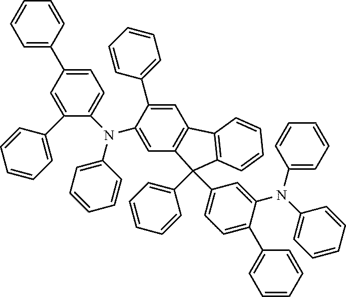 [SMILES: c1ccc(-c2ccc(N(c3ccccc3)c3cc4c(cc3-c3ccccc3)-c3ccccc3C4(c3ccccc3)c3ccc(-c4ccccc4)c(N(c4ccccc4)c4ccccc4)c3)c(-c3ccccc3)c2)cc1]

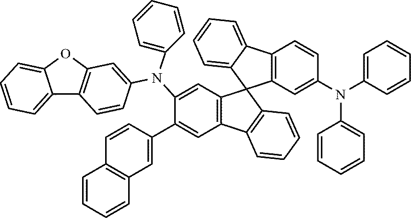 [SMILES: c1ccc(N(c2ccccc2)c2ccc3c(c2)C2(c4ccccc4-3)c3ccccc3-c3cc(-c4ccc5ccccc5c4)c(N(c4ccccc4)c4ccc5c(c4)oc4ccccc45)cc32)cc1]

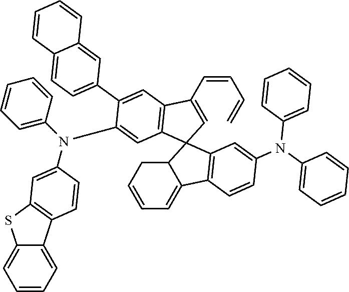 [SMILES: C=C/C=C\C1=CC2(c3cc(N(c4ccccc4)c4ccc5c(c4)sc4ccccc45)c(-c4ccc5ccccc5c4)cc31)c1cc(N(c3ccccc3)c3ccccc3)ccc1C1=CC=CCC12]

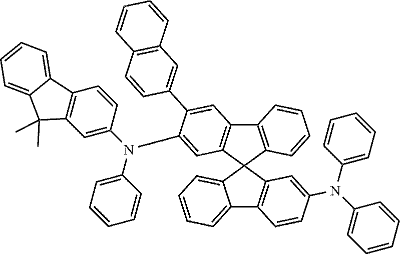 [SMILES: CC1(C)c2ccccc2-c2ccc(N(c3ccccc3)c3cc4c(cc3-c3ccc5ccccc5c3)-c3ccccc3C43c4ccccc4-c4ccc(N(c5ccccc5)c5ccccc5)cc43)cc21]

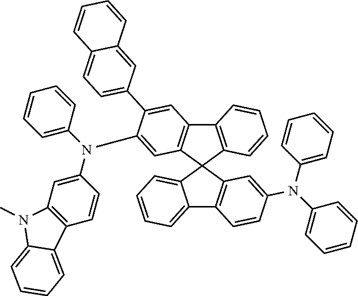 [SMILES: Cn1c2ccccc2c2ccc(N(c3ccccc3)c3cc4c(cc3-c3ccc5ccccc5c3)-c3ccccc3C43c4ccccc4-c4ccc(N(c5ccccc5)c5ccccc5)cc43)cc21]

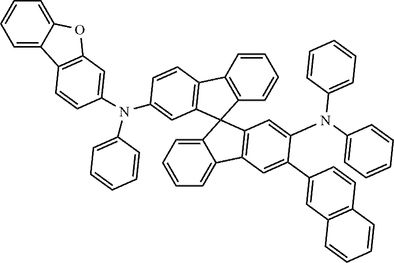 [SMILES: c1ccc(N(c2ccc3c(c2)C2(c4ccccc4-3)c3ccccc3-c3cc(-c4ccc5ccccc5c4)c(N(c4ccccc4)c4ccccc4)cc32)c2ccc3c(c2)oc2ccccc23)cc1]

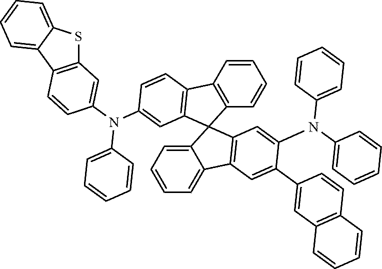 [SMILES: c1ccc(N(c2ccc3c(c2)C2(c4ccccc4-3)c3ccccc3-c3cc(-c4ccc5ccccc5c4)c(N(c4ccccc4)c4ccccc4)cc32)c2ccc3c(c2)sc2ccccc23)cc1]

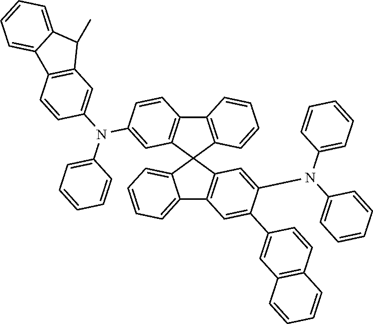 [SMILES: CC1c2ccccc2-c2ccc(N(c3ccccc3)c3ccc4c(c3)C3(c5ccccc5-4)c4ccccc4-c4cc(-c5ccc6ccccc6c5)c(N(c5ccccc5)c5ccccc5)cc43)cc21]

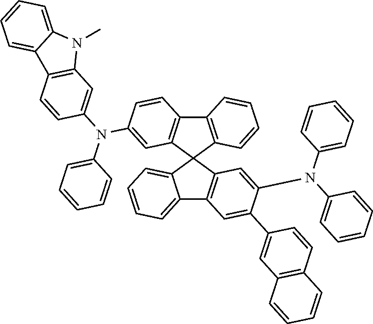 [SMILES: Cn1c2ccccc2c2ccc(N(c3ccccc3)c3ccc4c(c3)C3(c5ccccc5-4)c4ccccc4-c4cc(-c5ccc6ccccc6c5)c(N(c5ccccc5)c5ccccc5)cc43)cc21]

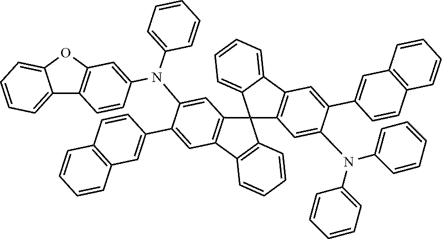 [SMILES: c1ccc(N(c2ccccc2)c2cc3c(cc2-c2ccc4ccccc4c2)-c2ccccc2C32c3ccccc3-c3cc(-c4ccc5ccccc5c4)c(N(c4ccccc4)c4ccc5c(c4)oc4ccccc45)cc32)cc1]

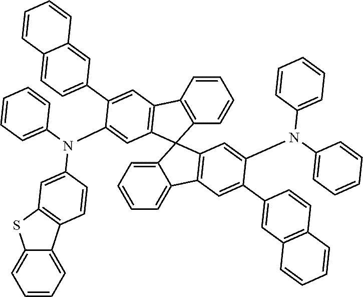 [SMILES: c1ccc(N(c2ccccc2)c2cc3c(cc2-c2ccc4ccccc4c2)-c2ccccc2C32c3ccccc3-c3cc(-c4ccc5ccccc5c4)c(N(c4ccccc4)c4ccc5c(c4)sc4ccccc45)cc32)cc1]

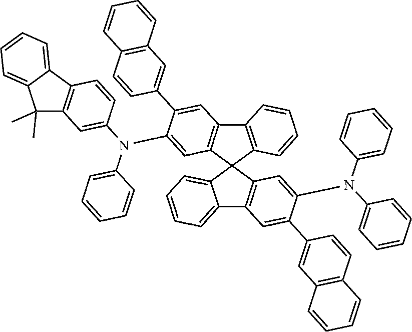 [SMILES: CC1(C)c2ccccc2-c2ccc(N(c3ccccc3)c3cc4c(cc3-c3ccc5ccccc5c3)-c3ccccc3C43c4ccccc4-c4cc(-c5ccc6ccccc6c5)c(N(c5ccccc5)c5ccccc5)cc43)cc21]

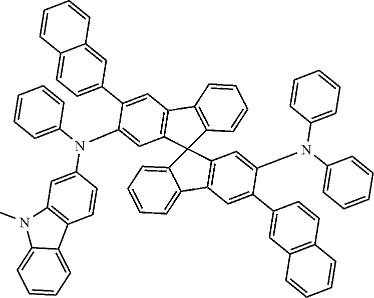 [SMILES: Cn1c2ccccc2c2ccc(N(c3ccccc3)c3cc4c(cc3-c3ccc5ccccc5c3)-c3ccccc3C43c4ccccc4-c4cc(-c5ccc6ccccc6c5)c(N(c5ccccc5)c5ccccc5)cc43)cc21]

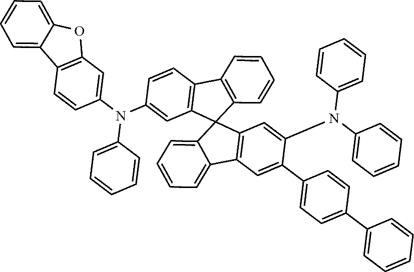 [SMILES: c1ccc(-c2ccc(-c3cc4c(cc3N(c3ccccc3)c3ccccc3)C3(c5ccccc5-c5ccc(N(c6ccccc6)c6ccc7c(c6)oc6ccccc67)cc53)c3ccccc3-4)cc2)cc1]

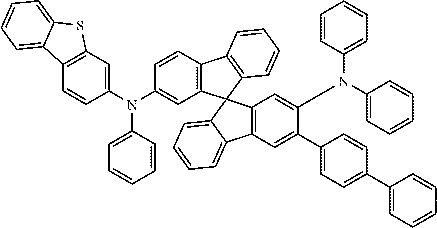 [SMILES: c1ccc(-c2ccc(-c3cc4c(cc3N(c3ccccc3)c3ccccc3)C3(c5ccccc5-c5ccc(N(c6ccccc6)c6ccc7c(c6)sc6ccccc67)cc53)c3ccccc3-4)cc2)cc1]

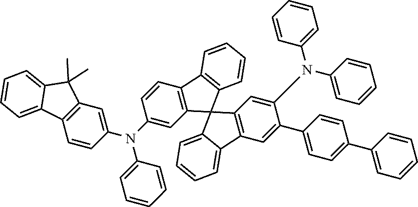 [SMILES: CC1(C)c2ccccc2-c2ccc(N(c3ccccc3)c3ccc4c(c3)C3(c5ccccc5-4)c4ccccc4-c4cc(-c5ccc(-c6ccccc6)cc5)c(N(c5ccccc5)c5ccccc5)cc43)cc21]

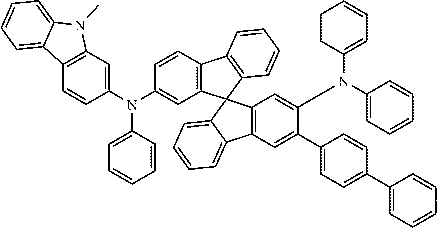 [SMILES: Cn1c2ccccc2c2ccc(N(c3ccccc3)c3ccc4c(c3)C3(c5ccccc5-4)c4ccccc4-c4cc(-c5ccc(-c6ccccc6)cc5)c(N(C5=CC=CCC5)c5ccccc5)cc43)cc21]

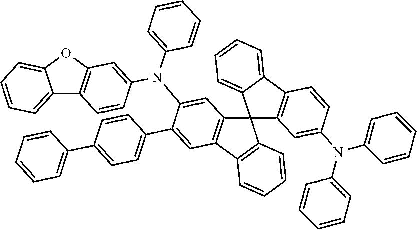 [SMILES: c1ccc(-c2ccc(-c3cc4c(cc3N(c3ccccc3)c3ccc5c(c3)oc3ccccc35)C3(c5ccccc5-c5ccc(N(c6ccccc6)c6ccccc6)cc53)c3ccccc3-4)cc2)cc1]

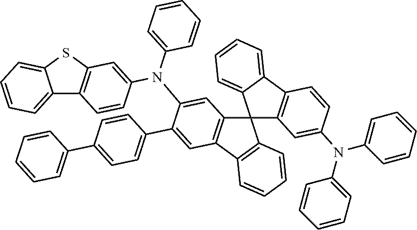 [SMILES: c1ccc(-c2ccc(-c3cc4c(cc3N(c3ccccc3)c3ccc5c(c3)sc3ccccc35)C3(c5ccccc5-c5ccc(N(c6ccccc6)c6ccccc6)cc53)c3ccccc3-4)cc2)cc1]

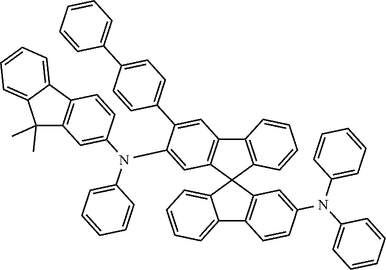 [SMILES: CC1(C)c2ccccc2-c2ccc(N(c3ccccc3)c3cc4c(cc3-c3ccc(-c5ccccc5)cc3)-c3ccccc3C43c4ccccc4-c4ccc(N(c5ccccc5)c5ccccc5)cc43)cc21]

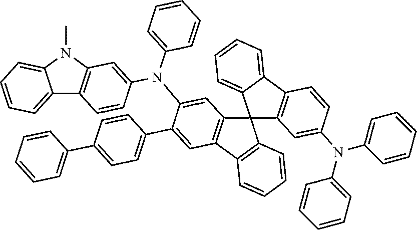 [SMILES: Cn1c2ccccc2c2ccc(N(c3ccccc3)c3cc4c(cc3-c3ccc(-c5ccccc5)cc3)-c3ccccc3C43c4ccccc4-c4ccc(N(c5ccccc5)c5ccccc5)cc43)cc21]

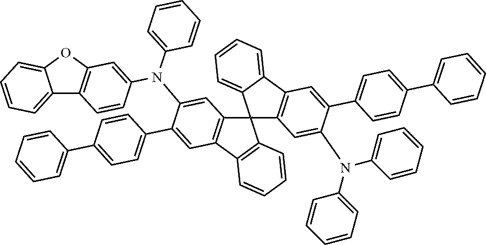 [SMILES: c1ccc(-c2ccc(-c3cc4c(cc3N(c3ccccc3)c3ccccc3)C3(c5ccccc5-4)c4ccccc4-c4cc(-c5ccc(-c6ccccc6)cc5)c(N(c5ccccc5)c5ccc6c(c5)oc5ccccc56)cc43)cc2)cc1]

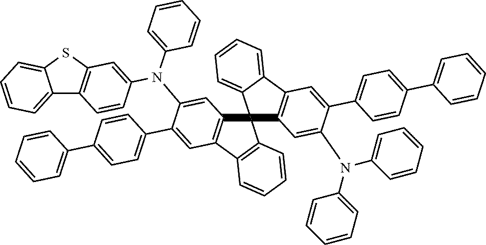 [SMILES: c1ccc(-c2ccc(-c3cc4c(cc3N(c3ccccc3)c3ccccc3)[C@@]3(c5ccccc5-4)c4ccccc4-c4cc(-c5ccc(-c6ccccc6)cc5)c(N(c5ccccc5)c5ccc6c(c5)sc5ccccc56)cc43)cc2)cc1]

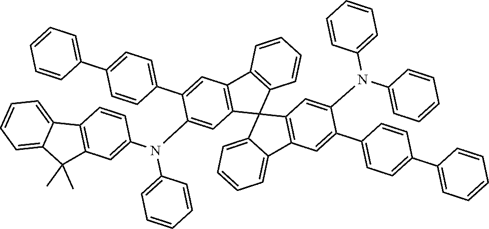 [SMILES: CC1(C)c2ccccc2-c2ccc(N(c3ccccc3)c3cc4c(cc3-c3ccc(-c5ccccc5)cc3)-c3ccccc3C43c4ccccc4-c4cc(-c5ccc(-c6ccccc6)cc5)c(N(c5ccccc5)c5ccccc5)cc43)cc21]

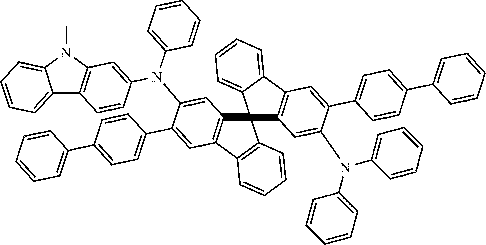 [SMILES: Cn1c2ccccc2c2ccc(N(c3ccccc3)c3cc4c(cc3-c3ccc(-c5ccccc5)cc3)-c3ccccc3[C@@]43c4ccccc4-c4cc(-c5ccc(-c6ccccc6)cc5)c(N(c5ccccc5)c5ccccc5)cc43)cc21]

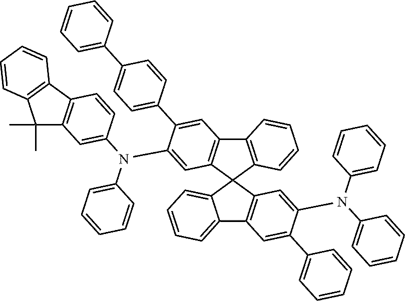 [SMILES: CC1(C)c2ccccc2-c2ccc(N(c3ccccc3)c3cc4c(cc3-c3ccc(-c5ccccc5)cc3)-c3ccccc3C43c4ccccc4-c4cc(-c5ccccc5)c(N(c5ccccc5)c5ccccc5)cc43)cc21]

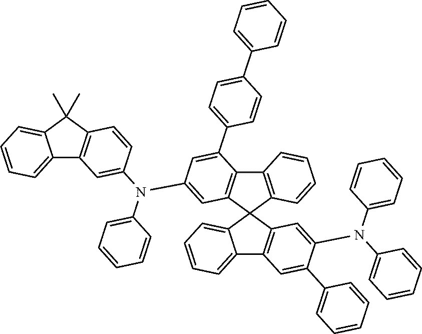 [SMILES: CC1(C)c2ccccc2-c2cc(N(c3ccccc3)c3cc(-c4ccc(-c5ccccc5)cc4)c4c(c3)C3(c5ccccc5-c5cc(-c6ccccc6)c(N(c6ccccc6)c6ccccc6)cc53)c3ccccc3-4)ccc21]

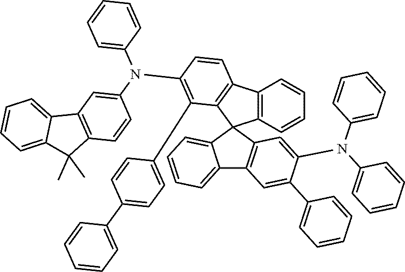 [SMILES: CC1(C)c2ccccc2-c2cc(N(c3ccccc3)c3ccc4c(c3-c3ccc(-c5ccccc5)cc3)C3(c5ccccc5-c5cc(-c6ccccc6)c(N(c6ccccc6)c6ccccc6)cc53)c3ccccc3-4)ccc21]

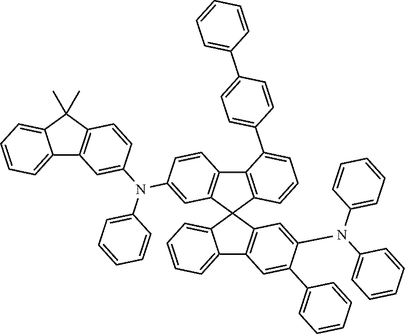 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3cccc(-c5ccc(-c6ccccc6)cc5)c3-4)ccc21]

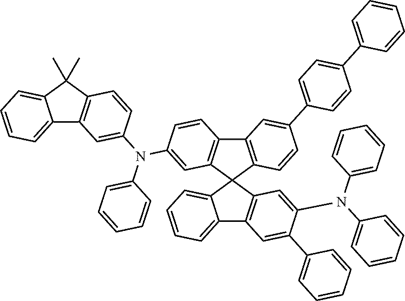 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3ccc(-c5ccc(-c6ccccc6)cc5)cc3-4)ccc21]

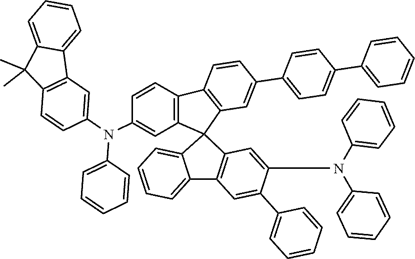 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3cc(-c5ccc(-c6ccccc6)cc5)ccc3-4)ccc21]

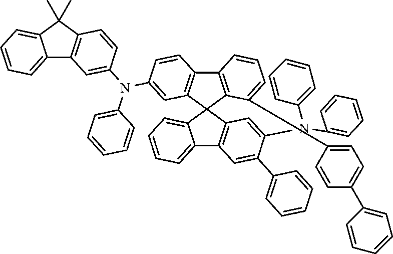 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3c(-c5ccc(-c6ccccc6)cc5)cccc3-4)ccc21]

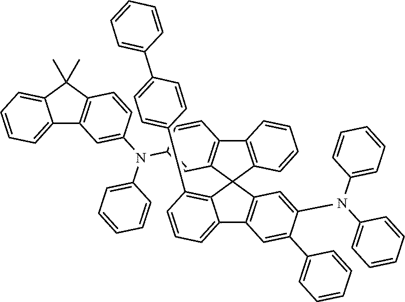 [SMILES: CC1(C)c2ccccc2-c2cc(N(c3ccccc3)c3ccc4c(c3)C3(c5ccccc5-4)c4cc(N(c5ccccc5)c5ccccc5)c(-c5ccccc5)cc4-c4cccc(-c5ccc(-c6ccccc6)cc5)c43)ccc21]

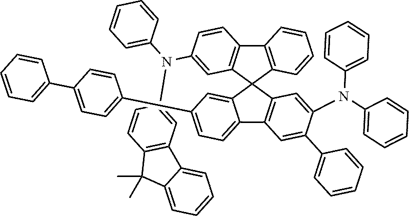 [SMILES: CC1(C)c2ccccc2-c2cc(N(c3ccccc3)c3ccc4c(c3)C3(c5ccccc5-4)c4cc(-c5ccc(-c6ccccc6)cc5)ccc4-c4cc(-c5ccccc5)c(N(c5ccccc5)c5ccccc5)cc43)ccc21]

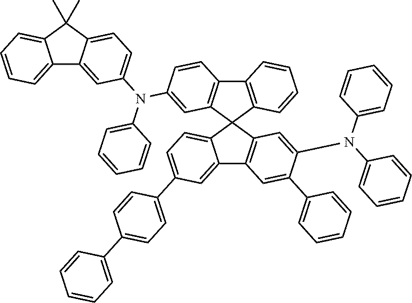 [SMILES: CC1(C)c2ccccc2-c2cc(N(c3ccccc3)c3ccc4c(c3)C3(c5ccccc5-4)c4ccc(-c5ccc(-c6ccccc6)cc5)cc4-c4cc(-c5ccccc5)c(N(c5ccccc5)c5ccccc5)cc43)ccc21]

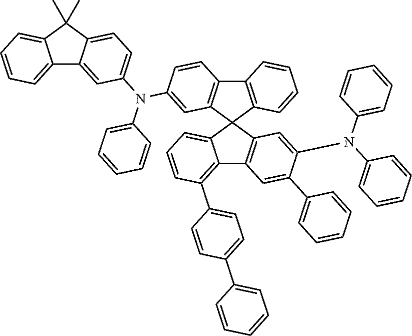 [SMILES: CC1(C)c2ccccc2-c2cc(N(c3ccccc3)c3ccc4c(c3)C3(c5ccccc5-4)c4cc(N(c5ccccc5)c5ccccc5)c(-c5ccccc5)cc4-c4c(-c5ccc(-c6ccccc6)cc5)cccc43)ccc21]

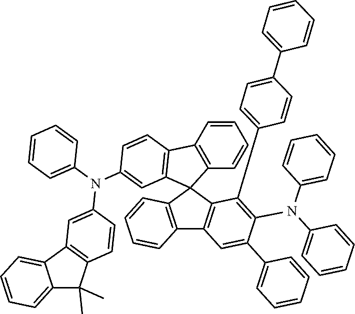 [SMILES: CC1(C)c2ccccc2-c2cc(N(c3ccccc3)c3ccc4c(c3)C3(c5ccccc5-4)c4ccccc4-c4cc(-c5ccccc5)c(N(c5ccccc5)c5ccccc5)c(-c5ccc(-c6ccccc6)cc5)c43)ccc21]

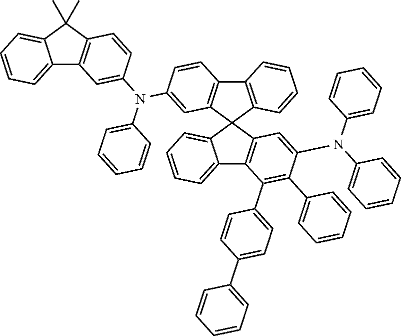 [SMILES: CC1(C)c2ccccc2-c2cc(N(c3ccccc3)c3ccc4c(c3)C3(c5ccccc5-4)c4ccccc4-c4c3cc(N(c3ccccc3)c3ccccc3)c(-c3ccccc3)c4-c3ccc(-c4ccccc4)cc3)ccc21]

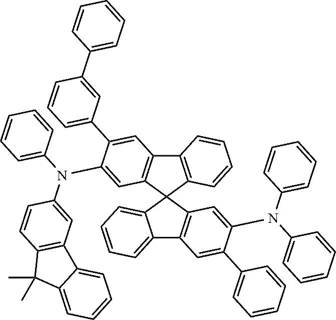 [SMILES: CC1(C)c2ccccc2-c2cc(N(c3ccccc3)c3cc4c(cc3-c3cccc(-c5ccccc5)c3)-c3ccccc3C43c4ccccc4-c4cc(-c5ccccc5)c(N(c5ccccc5)c5ccccc5)cc43)ccc21]

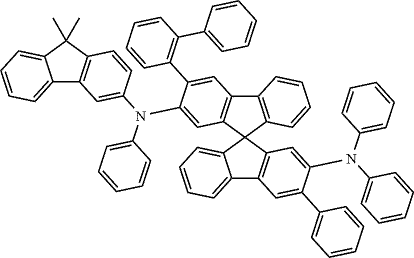 [SMILES: CC1(C)c2ccccc2-c2cc(N(c3ccccc3)c3cc4c(cc3-c3ccccc3-c3ccccc3)-c3ccccc3C43c4ccccc4-c4cc(-c5ccccc5)c(N(c5ccccc5)c5ccccc5)cc43)ccc21]

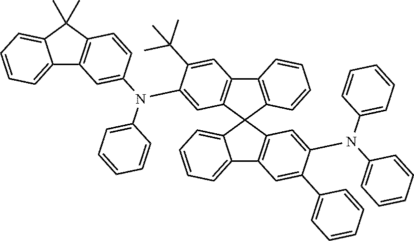 [SMILES: CC(C)(C)c1cc2c(cc1N(c1ccccc1)c1ccc3c(c1)-c1ccccc1C3(C)C)C1(c3ccccc3-c3cc(-c4ccccc4)c(N(c4ccccc4)c4ccccc4)cc31)c1ccccc1-2]

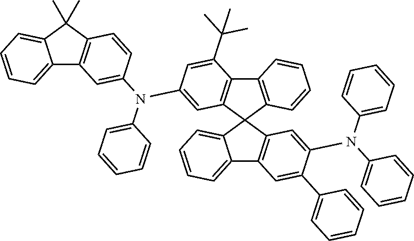 [SMILES: CC(C)(C)c1cc(N(c2ccccc2)c2ccc3c(c2)-c2ccccc2C3(C)C)cc2c1-c1ccccc1C21c2ccccc2-c2cc(-c3ccccc3)c(N(c3ccccc3)c3ccccc3)cc21]

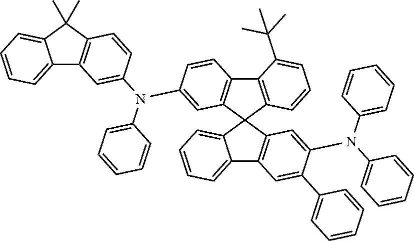 [SMILES: CC(C)(C)c1cccc2c1-c1ccc(N(c3ccccc3)c3ccc4c(c3)-c3ccccc3C4(C)C)cc1C21c2ccccc2-c2cc(-c3ccccc3)c(N(c3ccccc3)c3ccccc3)cc21]

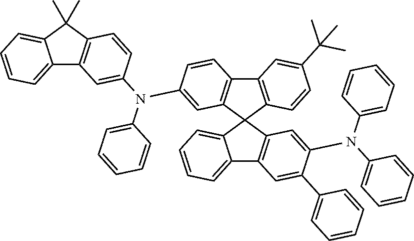 [SMILES: CC(C)(C)c1ccc2c(c1)-c1ccc(N(c3ccccc3)c3ccc4c(c3)-c3ccccc3C4(C)C)cc1C21c2ccccc2-c2cc(-c3ccccc3)c(N(c3ccccc3)c3ccccc3)cc21]

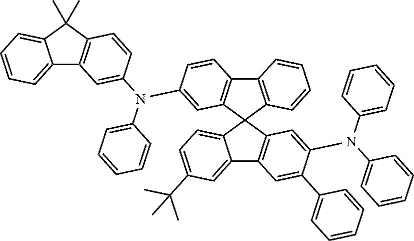 [SMILES: CC(C)(C)c1ccc2c(c1)-c1cc(-c3ccccc3)c(N(c3ccccc3)c3ccccc3)cc1C21c2ccccc2-c2ccc(N(c3ccccc3)c3ccc4c(c3)-c3ccccc3C4(C)C)cc21]

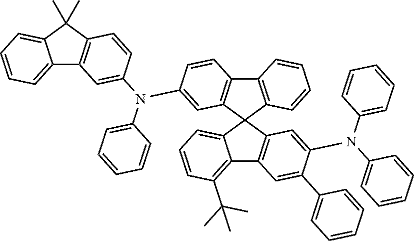 [SMILES: CC(C)(C)c1cccc2c1-c1cc(-c3ccccc3)c(N(c3ccccc3)c3ccccc3)cc1C21c2ccccc2-c2ccc(N(c3ccccc3)c3ccc4c(c3)-c3ccccc3C4(C)C)cc21]

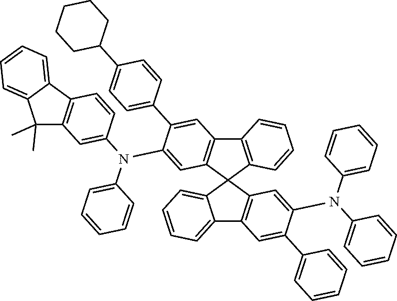 [SMILES: CC1(C)c2ccccc2-c2ccc(N(c3ccccc3)c3cc4c(cc3-c3ccc(C5CCCCC5)cc3)-c3ccccc3C43c4ccccc4-c4cc(-c5ccccc5)c(N(c5ccccc5)c5ccccc5)cc43)cc21]

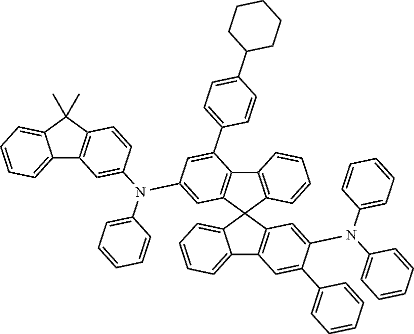 [SMILES: CC1(C)c2ccccc2-c2cc(N(c3ccccc3)c3cc(-c4ccc(C5CCCCC5)cc4)c4c(c3)C3(c5ccccc5-c5cc(-c6ccccc6)c(N(c6ccccc6)c6ccccc6)cc53)c3ccccc3-4)ccc21]

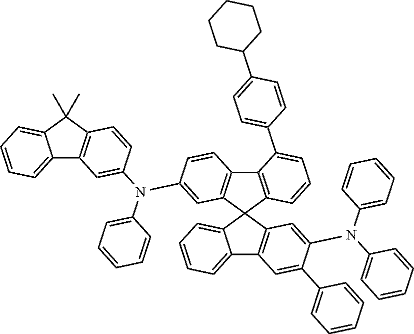 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3cccc(-c5ccc(C6CCCCC6)cc5)c3-4)ccc21]

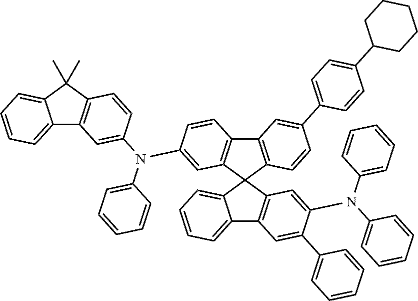 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3ccc(-c5ccc(C6CCCCC6)cc5)cc3-4)ccc21]

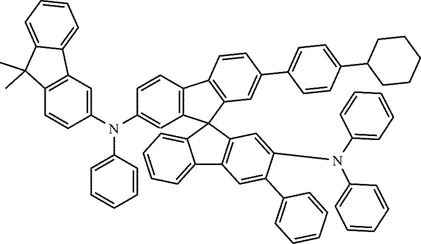 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3cc(-c5ccc(C6CCCCC6)cc5)ccc3-4)ccc21]

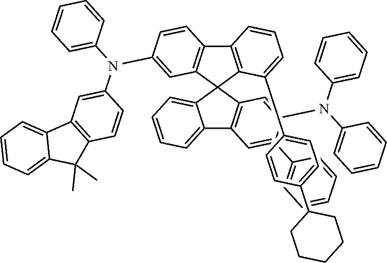 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3c(-c5ccc(C6CCCCC6)cc5)cccc3-4)ccc21]

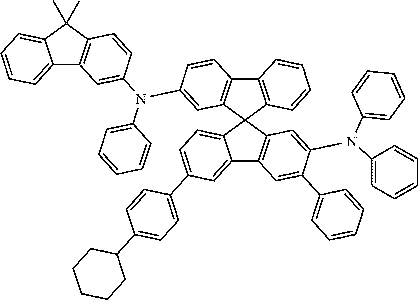 [SMILES: CC1(C)c2ccccc2-c2cc(N(c3ccccc3)c3ccc4c(c3)C3(c5ccccc5-4)c4ccc(-c5ccc(C6CCCCC6)cc5)cc4-c4cc(-c5ccccc5)c(N(c5ccccc5)c5ccccc5)cc43)ccc21]

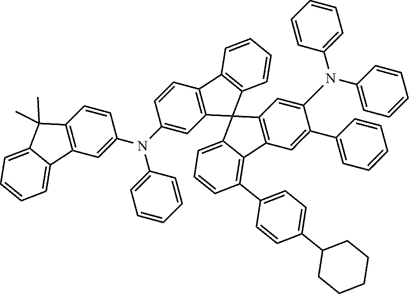 [SMILES: CC1(C)c2ccccc2-c2cc(N(c3ccccc3)c3ccc4c(c3)C3(c5ccccc5-4)c4cc(N(c5ccccc5)c5ccccc5)c(-c5ccccc5)cc4-c4c(-c5ccc(C6CCCCC6)cc5)cccc43)ccc21]

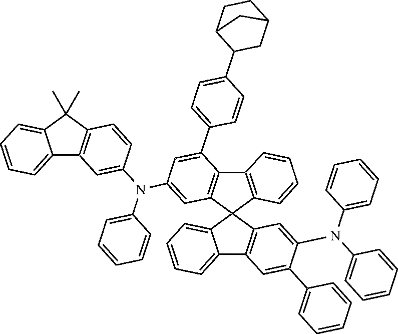 [SMILES: CC1(C)c2ccccc2-c2cc(N(c3ccccc3)c3cc(-c4ccc(C5CC6CCC5C6)cc4)c4c(c3)C3(c5ccccc5-c5cc(-c6ccccc6)c(N(c6ccccc6)c6ccccc6)cc53)c3ccccc3-4)ccc21]

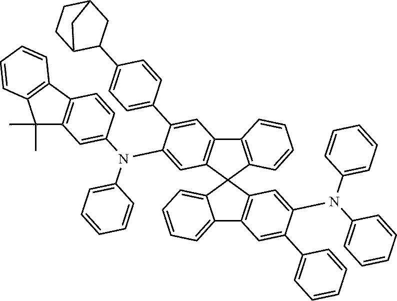 [SMILES: CC1(C)c2ccccc2-c2ccc(N(c3ccccc3)c3cc4c(cc3-c3ccc(C5CC6CCC5C6)cc3)-c3ccccc3C43c4ccccc4-c4cc(-c5ccccc5)c(N(c5ccccc5)c5ccccc5)cc43)cc21]

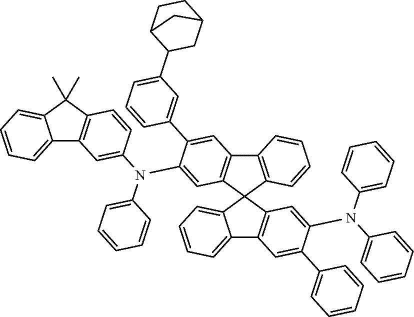 [SMILES: CC1(C)c2ccccc2-c2cc(N(c3ccccc3)c3cc4c(cc3-c3cccc(C5CC6CCC5C6)c3)-c3ccccc3C43c4ccccc4-c4cc(-c5ccccc5)c(N(c5ccccc5)c5ccccc5)cc43)ccc21]

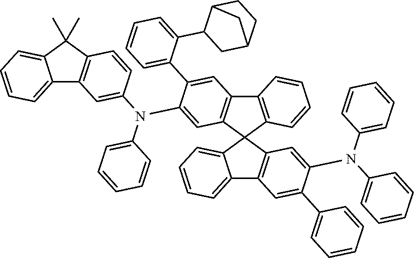 [SMILES: CC1(C)c2ccccc2-c2cc(N(c3ccccc3)c3cc4c(cc3-c3ccccc3C3CC5CCC3C5)-c3ccccc3C43c4ccccc4-c4cc(-c5ccccc5)c(N(c5ccccc5)c5ccccc5)cc43)ccc21]

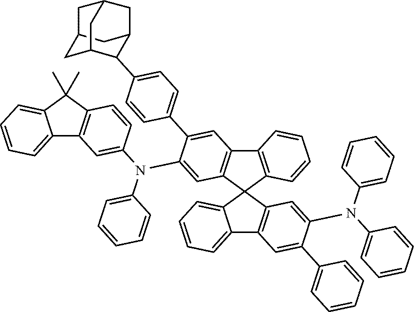 [SMILES: CC1(C)c2ccccc2-c2cc(N(c3ccccc3)c3cc4c(cc3-c3ccc(C5C6CC7CC(C6)CC5C7)cc3)-c3ccccc3C43c4ccccc4-c4cc(-c5ccccc5)c(N(c5ccccc5)c5ccccc5)cc43)ccc21]

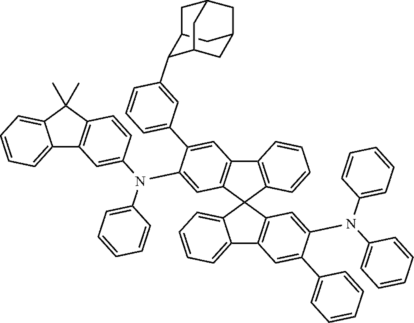 [SMILES: CC1(C)c2ccccc2-c2cc(N(c3ccccc3)c3cc4c(cc3-c3cccc(C5C6CC7CC(C6)CC5C7)c3)-c3ccccc3C43c4ccccc4-c4cc(-c5ccccc5)c(N(c5ccccc5)c5ccccc5)cc43)ccc21]

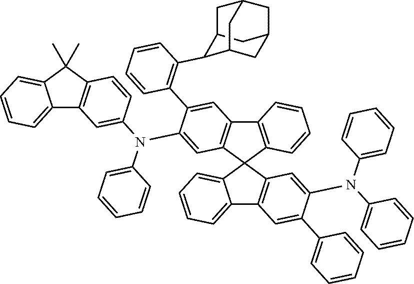 [SMILES: CC1(C)c2ccccc2-c2cc(N(c3ccccc3)c3cc4c(cc3-c3ccccc3C3C5CC6CC(C5)CC3C6)-c3ccccc3C43c4ccccc4-c4cc(-c5ccccc5)c(N(c5ccccc5)c5ccccc5)cc43)ccc21]

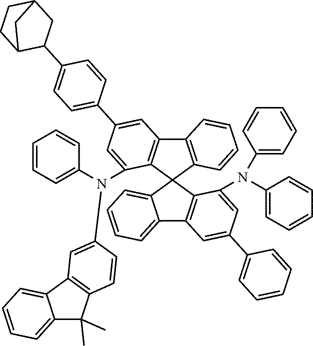 [SMILES: CC1(C)c2ccccc2-c2cc(N(c3ccccc3)c3cc(-c4ccc(C5CC6CCC5C6)cc4)cc4c3C3(c5ccccc5-c5cc(-c6ccccc6)cc(N(c6ccccc6)c6ccccc6)c53)c3ccccc3-4)ccc21]

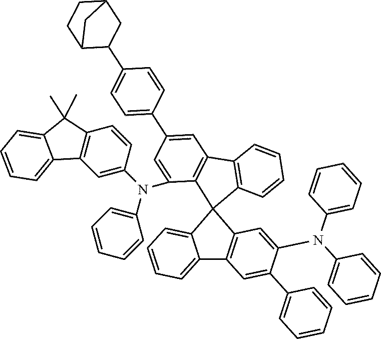 [SMILES: CC1(C)c2ccccc2-c2cc(N(c3ccccc3)c3cc(-c4ccc(C5CC6CCC5C6)cc4)cc4c3C3(c5ccccc5-c5cc(-c6ccccc6)c(N(c6ccccc6)c6ccccc6)cc53)c3ccccc3-4)ccc21]

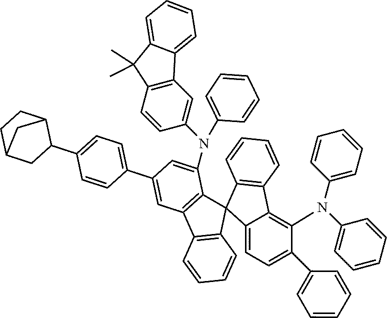 [SMILES: CC1(C)c2ccccc2-c2cc(N(c3ccccc3)c3cc(-c4ccc(C5CC6CCC5C6)cc4)cc4c3C3(c5ccccc5-4)c4ccccc4-c4c3ccc(-c3ccccc3)c4N(c3ccccc3)c3ccccc3)ccc21]

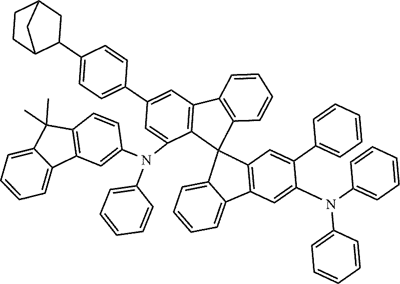 [SMILES: CC1(C)c2ccccc2-c2cc(N(c3ccccc3)c3cc(-c4ccc(C5CC6CCC5C6)cc4)cc4c3C3(c5ccccc5-c5cc(N(c6ccccc6)c6ccccc6)c(-c6ccccc6)cc53)c3ccccc3-4)ccc21]

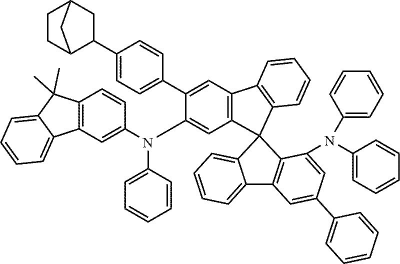 [SMILES: CC1(C)c2ccccc2-c2cc(N(c3ccccc3)c3cc4c(cc3-c3ccc(C5CC6CCC5C6)cc3)-c3ccccc3C43c4ccccc4-c4cc(-c5ccccc5)cc(N(c5ccccc5)c5ccccc5)c43)ccc21]

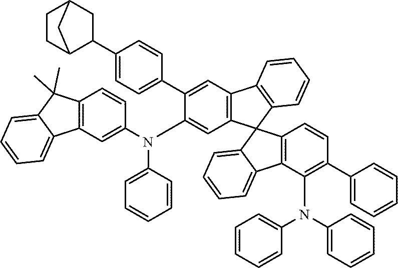 [SMILES: CC1(C)c2ccccc2-c2cc(N(c3ccccc3)c3cc4c(cc3-c3ccc(C5CC6CCC5C6)cc3)-c3ccccc3C43c4ccccc4-c4c3ccc(-c3ccccc3)c4N(c3ccccc3)c3ccccc3)ccc21]

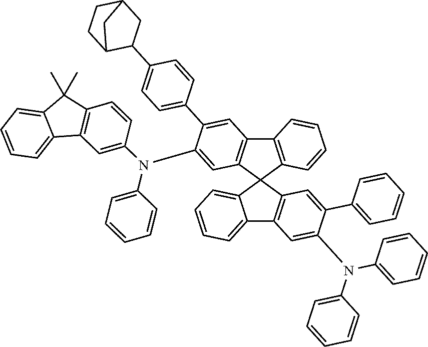 [SMILES: CC1(C)c2ccccc2-c2cc(N(c3ccccc3)c3cc4c(cc3-c3ccc(C5CC6CCC5C6)cc3)-c3ccccc3C43c4ccccc4-c4cc(N(c5ccccc5)c5ccccc5)c(-c5ccccc5)cc43)ccc21]

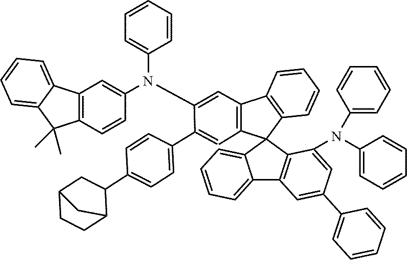 [SMILES: CC1(C)c2ccccc2-c2cc(N(c3ccccc3)c3cc4c(cc3-c3ccc(C5CC6CCC5C6)cc3)C3(c5ccccc5-4)c4ccccc4-c4cc(-c5ccccc5)cc(N(c5ccccc5)c5ccccc5)c43)ccc21]

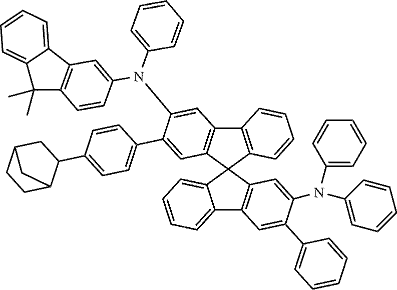 [SMILES: CC1(C)c2ccccc2-c2cc(N(c3ccccc3)c3cc4c(cc3-c3ccc(C5CC6CCC5C6)cc3)C3(c5ccccc5-4)c4ccccc4-c4cc(-c5ccccc5)c(N(c5ccccc5)c5ccccc5)cc43)ccc21]

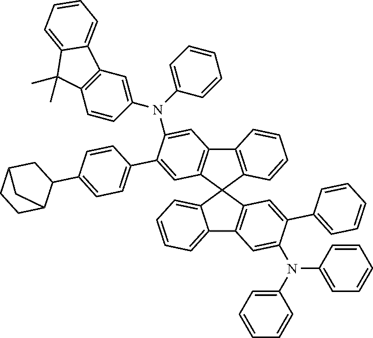 [SMILES: CC1(C)c2ccccc2-c2cc(N(c3ccccc3)c3cc4c(cc3-c3ccc(C5CC6CCC5C6)cc3)C3(c5ccccc5-c5cc(N(c6ccccc6)c6ccccc6)c(-c6ccccc6)cc53)c3ccccc3-4)ccc21]

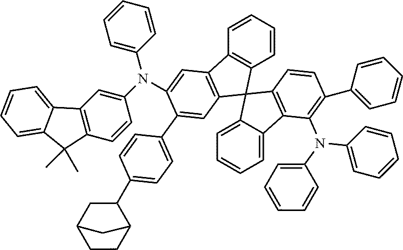 [SMILES: CC1(C)c2ccccc2-c2cc(N(c3ccccc3)c3cc4c(cc3-c3ccc(C5CC6CCC5C6)cc3)C3(c5ccccc5-4)c4ccccc4-c4c3ccc(-c3ccccc3)c4N(c3ccccc3)c3ccccc3)ccc21]

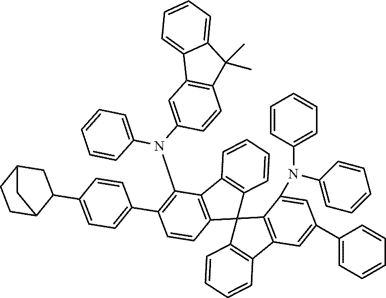 [SMILES: CC1(C)c2ccccc2-c2cc(N(c3ccccc3)c3c(-c4ccc(C5CC6CCC5C6)cc4)ccc4c3-c3ccccc3C43c4ccccc4-c4cc(-c5ccccc5)cc(N(c5ccccc5)c5ccccc5)c43)ccc21]

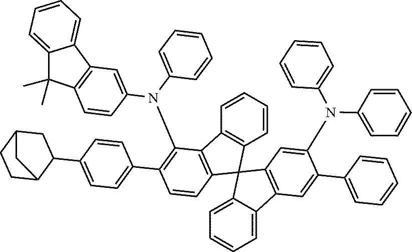 [SMILES: CC1(C)c2ccccc2-c2cc(N(c3ccccc3)c3c(-c4ccc(C5CC6CCC5C6)cc4)ccc4c3-c3ccccc3C43c4ccccc4-c4cc(-c5ccccc5)c(N(c5ccccc5)c5ccccc5)cc43)ccc21]

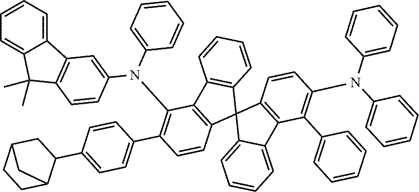 [SMILES: CC1(C)c2ccccc2-c2cc(N(c3ccccc3)c3c(-c4ccc(C5CC6CCC5C6)cc4)ccc4c3-c3ccccc3C43c4ccccc4-c4c3ccc(N(c3ccccc3)c3ccccc3)c4-c3ccccc3)ccc21]

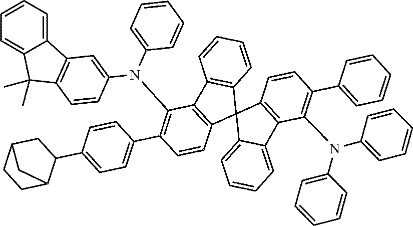 [SMILES: CC1(C)c2ccccc2-c2cc(N(c3ccccc3)c3c(-c4ccc(C5CC6CCC5C6)cc4)ccc4c3-c3ccccc3C43c4ccccc4-c4c3ccc(-c3ccccc3)c4N(c3ccccc3)c3ccccc3)ccc21]

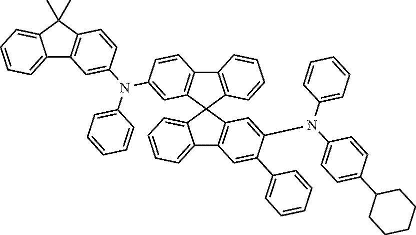 [SMILES: CC1(C)c2ccccc2-c2cc(N(c3ccccc3)c3ccc4c(c3)C3(c5ccccc5-4)c4ccccc4-c4cc(-c5ccccc5)c(N(c5ccccc5)c5ccc(C6CCCCC6)cc5)cc43)ccc21]

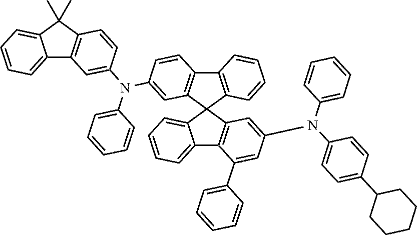 [SMILES: CC1(C)c2ccccc2-c2cc(N(c3ccccc3)c3ccc4c(c3)C3(c5ccccc5-4)c4ccccc4-c4c(-c5ccccc5)cc(N(c5ccccc5)c5ccc(C6CCCCC6)cc5)cc43)ccc21]

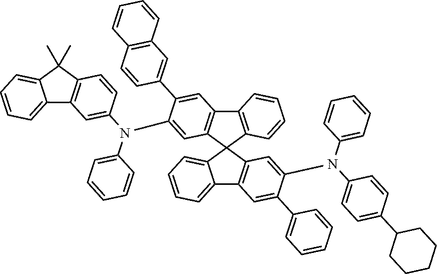 [SMILES: CC1(C)c2ccccc2-c2cc(N(c3ccccc3)c3cc4c(cc3-c3ccc5ccccc5c3)-c3ccccc3C43c4ccccc4-c4cc(-c5ccccc5)c(N(c5ccccc5)c5ccc(C6CCCCC6)cc5)cc43)ccc21]

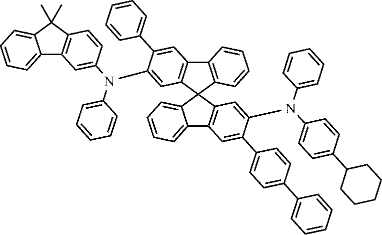 [SMILES: CC1(C)c2ccccc2-c2cc(N(c3ccccc3)c3cc4c(cc3-c3ccccc3)-c3ccccc3C43c4ccccc4-c4cc(-c5ccc(-c6ccccc6)cc5)c(N(c5ccccc5)c5ccc(C6CCCCC6)cc5)cc43)ccc21]

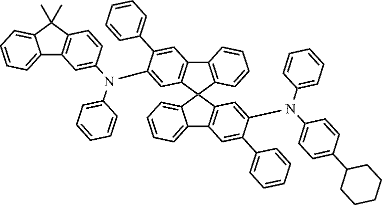 [SMILES: CC1(C)c2ccccc2-c2cc(N(c3ccccc3)c3cc4c(cc3-c3ccccc3)-c3ccccc3C43c4ccccc4-c4cc(-c5ccccc5)c(N(c5ccccc5)c5ccc(C6CCCCC6)cc5)cc43)ccc21]

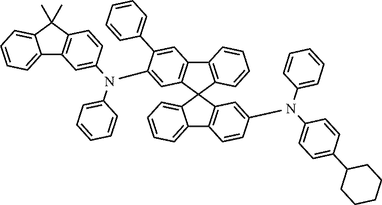 [SMILES: CC1(C)c2ccccc2-c2cc(N(c3ccccc3)c3cc4c(cc3-c3ccccc3)-c3ccccc3C43c4ccccc4-c4ccc(N(c5ccccc5)c5ccc(C6CCCCC6)cc5)cc43)ccc21]

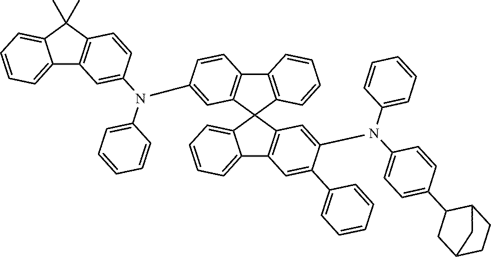 [SMILES: CC1(C)c2ccccc2-c2cc(N(c3ccccc3)c3ccc4c(c3)C3(c5ccccc5-4)c4ccccc4-c4cc(-c5ccccc5)c(N(c5ccccc5)c5ccc(C6CC7CCC6C7)cc5)cc43)ccc21]

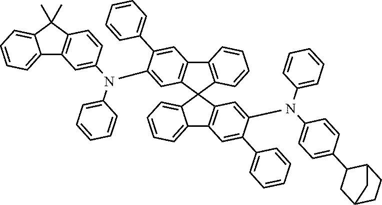 [SMILES: CC1(C)c2ccccc2-c2cc(N(c3ccccc3)c3cc4c(cc3-c3ccccc3)-c3ccccc3C43c4ccccc4-c4cc(-c5ccccc5)c(N(c5ccccc5)c5ccc(C6CC7CCC6C7)cc5)cc43)ccc21]

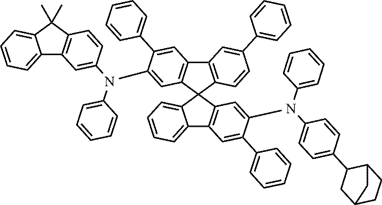 [SMILES: CC1(C)c2ccccc2-c2cc(N(c3ccccc3)c3cc4c(cc3-c3ccccc3)-c3cc(-c5ccccc5)ccc3C43c4ccccc4-c4cc(-c5ccccc5)c(N(c5ccccc5)c5ccc(C6CC7CCC6C7)cc5)cc43)ccc21]

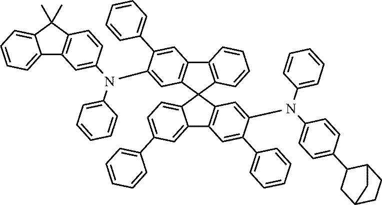 [SMILES: CC1(C)c2ccccc2-c2cc(N(c3ccccc3)c3cc4c(cc3-c3ccccc3)-c3ccccc3C43c4ccc(-c5ccccc5)cc4-c4cc(-c5ccccc5)c(N(c5ccccc5)c5ccc(C6CC7CCC6C7)cc5)cc43)ccc21]

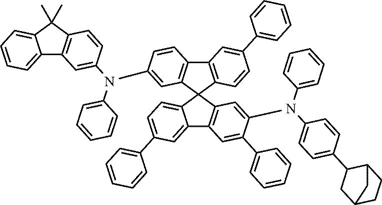 [SMILES: CC1(C)c2ccccc2-c2cc(N(c3ccccc3)c3ccc4c(c3)C3(c5ccc(-c6ccccc6)cc5-4)c4ccc(-c5ccccc5)cc4-c4cc(-c5ccccc5)c(N(c5ccccc5)c5ccc(C6CC7CCC6C7)cc5)cc43)ccc21]

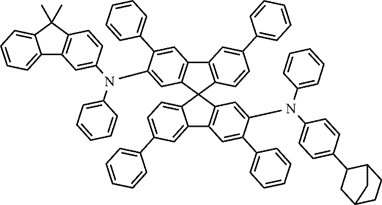 [SMILES: CC1(C)c2ccccc2-c2cc(N(c3ccccc3)c3cc4c(cc3-c3ccccc3)-c3cc(-c5ccccc5)ccc3C43c4ccc(-c5ccccc5)cc4-c4cc(-c5ccccc5)c(N(c5ccccc5)c5ccc(C6CC7CCC6C7)cc5)cc43)ccc21]

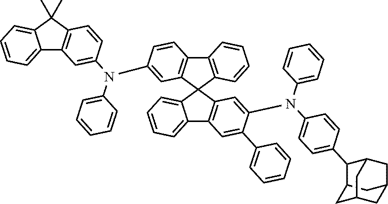 [SMILES: CC1(C)c2ccccc2-c2cc(N(c3ccccc3)c3ccc4c(c3)C3(c5ccccc5-4)c4ccccc4-c4cc(-c5ccccc5)c(N(c5ccccc5)c5ccc(C6C7CC8CC(C7)CC6C8)cc5)cc43)ccc21]

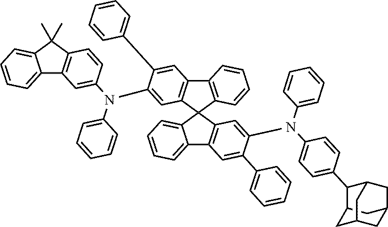 [SMILES: CC1(C)c2ccccc2-c2cc(N(c3ccccc3)c3cc4c(cc3-c3ccccc3)-c3ccccc3C43c4ccccc4-c4cc(-c5ccccc5)c(N(c5ccccc5)c5ccc(C6C7CC8CC(C7)CC6C8)cc5)cc43)ccc21]

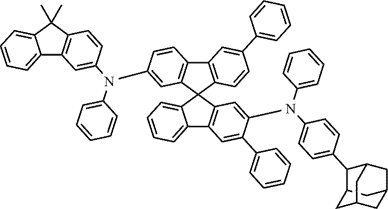 [SMILES: CC1(C)c2ccccc2-c2cc(N(c3ccccc3)c3ccc4c(c3)C3(c5ccccc5-c5cc(-c6ccccc6)c(N(c6ccccc6)c6ccc(C7C8CC9CC(C8)CC7C9)cc6)cc53)c3ccc(-c5ccccc5)cc3-4)ccc21]

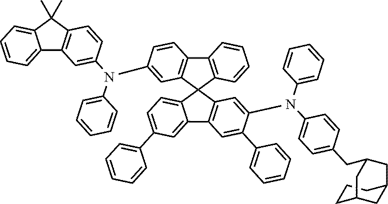 [SMILES: CC1(C)c2ccccc2-c2cc(N(c3ccccc3)c3ccc4c(c3)C3(c5ccccc5-4)c4ccc(-c5ccccc5)cc4-c4cc(-c5ccccc5)c(N(c5ccccc5)c5ccc(CC6CC7CCCC(C7)C6)cc5)cc43)ccc21]

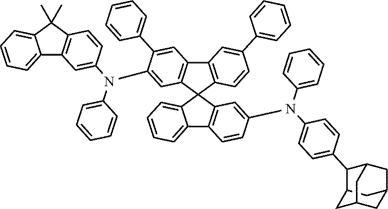 [SMILES: CC1(C)c2ccccc2-c2cc(N(c3ccccc3)c3cc4c(cc3-c3ccccc3)-c3cc(-c5ccccc5)ccc3C43c4ccccc4-c4ccc(N(c5ccccc5)c5ccc(C6C7CC8CC(C7)CC6C8)cc5)cc43)ccc21]

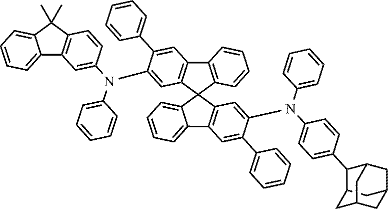 [SMILES: CC1(C)c2ccccc2-c2cc(N(c3ccccc3)c3cc4c(cc3-c3ccccc3)-c3ccccc3C43c4ccccc4-c4cc(-c5ccccc5)c(N(c5ccccc5)c5ccc(C6C7CC8CC(C7)CC6C8)cc5)cc43)ccc21]

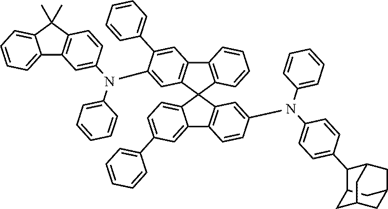 [SMILES: CC1(C)c2ccccc2-c2cc(N(c3ccccc3)c3cc4c(cc3-c3ccccc3)-c3ccccc3C43c4ccc(-c5ccccc5)cc4-c4ccc(N(c5ccccc5)c5ccc(C6C7CC8CC(C7)CC6C8)cc5)cc43)ccc21]

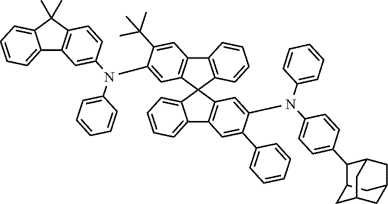 [SMILES: CC(C)(C)c1cc2c(cc1N(c1ccccc1)c1ccc3c(c1)-c1ccccc1C3(C)C)C1(c3ccccc3-c3cc(-c4ccccc4)c(N(c4ccccc4)c4ccc(C5C6CC7CC(C6)CC5C7)cc4)cc31)c1ccccc1-2]

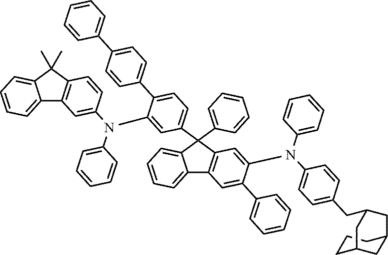 [SMILES: CC1(C)c2ccccc2-c2cc(N(c3ccccc3)c3cc(C4(c5ccccc5)c5ccccc5-c5cc(-c6ccccc6)c(N(c6ccccc6)c6ccc(CC7CC8CCCC(C8)C7)cc6)cc54)ccc3-c3ccc(-c4ccccc4)cc3)ccc21]

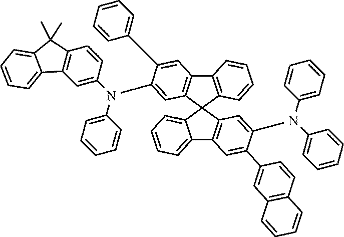 [SMILES: CC1(C)c2ccccc2-c2cc(N(c3ccccc3)c3cc4c(cc3-c3ccccc3)-c3ccccc3C43c4ccccc4-c4cc(-c5ccc6ccccc6c5)c(N(c5ccccc5)c5ccccc5)cc43)ccc21]

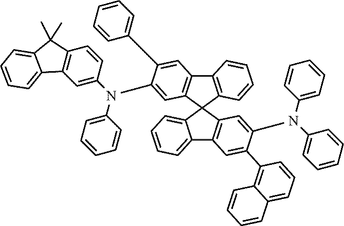 [SMILES: CC1(C)c2ccccc2-c2cc(N(c3ccccc3)c3cc4c(cc3-c3ccccc3)-c3ccccc3C43c4ccccc4-c4cc(-c5cccc6ccccc56)c(N(c5ccccc5)c5ccccc5)cc43)ccc21]

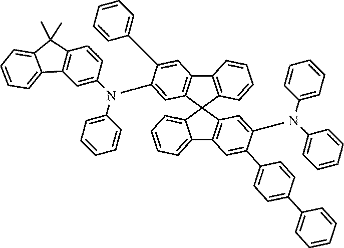 [SMILES: CC1(C)c2ccccc2-c2cc(N(c3ccccc3)c3cc4c(cc3-c3ccccc3)-c3ccccc3C43c4ccccc4-c4cc(-c5ccc(-c6ccccc6)cc5)c(N(c5ccccc5)c5ccccc5)cc43)ccc21]

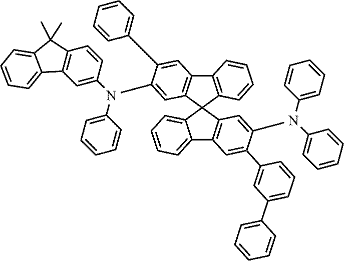 [SMILES: CC1(C)c2ccccc2-c2cc(N(c3ccccc3)c3cc4c(cc3-c3ccccc3)-c3ccccc3C43c4ccccc4-c4cc(-c5cccc(-c6ccccc6)c5)c(N(c5ccccc5)c5ccccc5)cc43)ccc21]

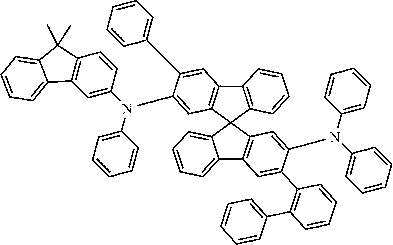 [SMILES: CC1(C)c2ccccc2-c2cc(N(c3ccccc3)c3cc4c(cc3-c3ccccc3)-c3ccccc3C43c4ccccc4-c4cc(-c5ccccc5-c5ccccc5)c(N(c5ccccc5)c5ccccc5)cc43)ccc21]

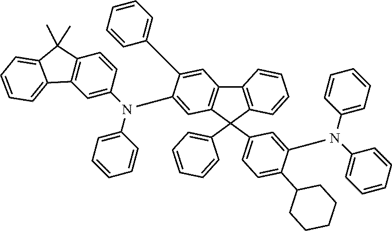 [SMILES: CC1(C)c2ccccc2-c2cc(N(c3ccccc3)c3cc4c(cc3-c3ccccc3)-c3ccccc3C4(c3ccccc3)c3ccc(C4CCCCC4)c(N(c4ccccc4)c4ccccc4)c3)ccc21]

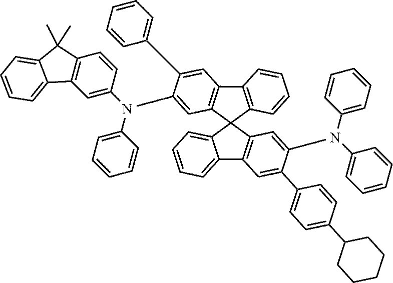 [SMILES: CC1(C)c2ccccc2-c2cc(N(c3ccccc3)c3cc4c(cc3-c3ccccc3)-c3ccccc3C43c4ccccc4-c4cc(-c5ccc(C6CCCCC6)cc5)c(N(c5ccccc5)c5ccccc5)cc43)ccc21]

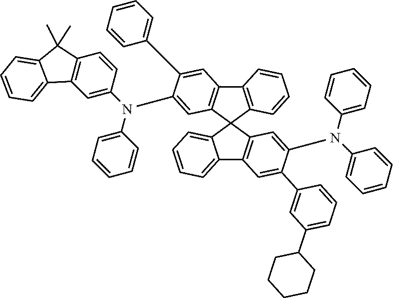 [SMILES: CC1(C)c2ccccc2-c2cc(N(c3ccccc3)c3cc4c(cc3-c3ccccc3)-c3ccccc3C43c4ccccc4-c4cc(-c5cccc(C6CCCCC6)c5)c(N(c5ccccc5)c5ccccc5)cc43)ccc21]

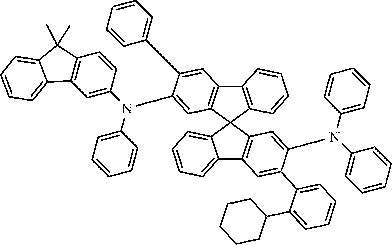 [SMILES: CC1(C)c2ccccc2-c2cc(N(c3ccccc3)c3cc4c(cc3-c3ccccc3)-c3ccccc3C43c4ccccc4-c4cc(-c5ccccc5C5CCCCC5)c(N(c5ccccc5)c5ccccc5)cc43)ccc21]

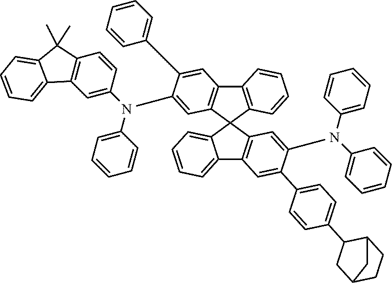 [SMILES: CC1(C)c2ccccc2-c2cc(N(c3ccccc3)c3cc4c(cc3-c3ccccc3)-c3ccccc3C43c4ccccc4-c4cc(-c5ccc(C6CC7CCC6C7)cc5)c(N(c5ccccc5)c5ccccc5)cc43)ccc21]

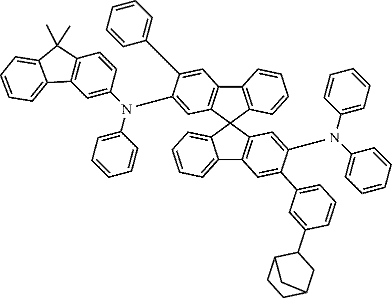 [SMILES: CC1(C)c2ccccc2-c2cc(N(c3ccccc3)c3cc4c(cc3-c3ccccc3)-c3ccccc3C43c4ccccc4-c4cc(-c5cccc(C6CC7CCC6C7)c5)c(N(c5ccccc5)c5ccccc5)cc43)ccc21]

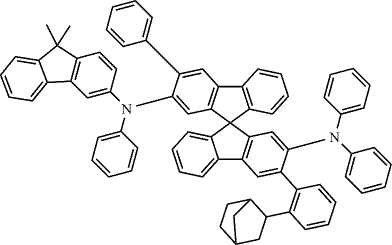 [SMILES: CC1(C)c2ccccc2-c2cc(N(c3ccccc3)c3cc4c(cc3-c3ccccc3)-c3ccccc3C43c4ccccc4-c4cc(-c5ccccc5C5CC6CCC5C6)c(N(c5ccccc5)c5ccccc5)cc43)ccc21]

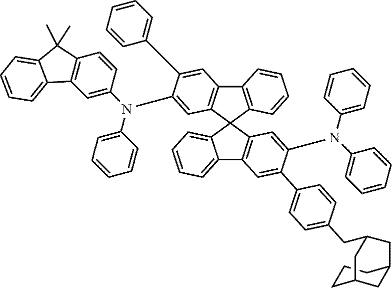 [SMILES: CC1(C)c2ccccc2-c2cc(N(c3ccccc3)c3cc4c(cc3-c3ccccc3)-c3ccccc3C43c4ccccc4-c4cc(-c5ccc(CC6CC7CCCC(C7)C6)cc5)c(N(c5ccccc5)c5ccccc5)cc43)ccc21]

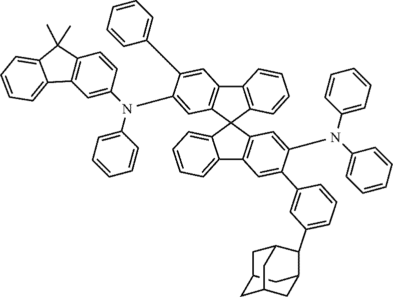 [SMILES: CC1(C)c2ccccc2-c2cc(N(c3ccccc3)c3cc4c(cc3-c3ccccc3)-c3ccccc3C43c4ccccc4-c4cc(-c5cccc(C6C7CC8CC(C7)CC6C8)c5)c(N(c5ccccc5)c5ccccc5)cc43)ccc21]

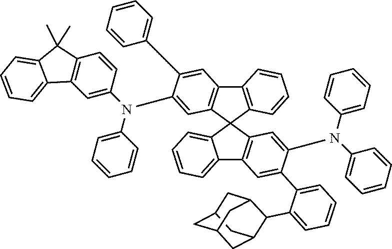 [SMILES: CC1(C)c2ccccc2-c2cc(N(c3ccccc3)c3cc4c(cc3-c3ccccc3)-c3ccccc3C43c4ccccc4-c4cc(-c5ccccc5C5C6CC7CC(C6)CC5C7)c(N(c5ccccc5)c5ccccc5)cc43)ccc21]

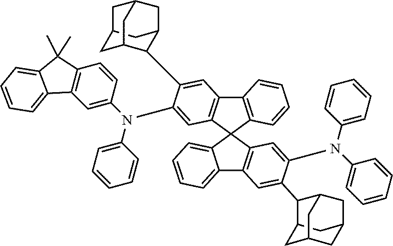 [SMILES: CC1(C)c2ccccc2-c2cc(N(c3ccccc3)c3cc4c(cc3C3C5CC6CC(C5)CC3C6)-c3ccccc3C43c4ccccc4-c4cc(C5C6CC7CC(C6)CC5C7)c(N(c5ccccc5)c5ccccc5)cc43)ccc21]

In an embodiment, the layer of the light-emitting device may be a hole transport layer.

In an embodiment, the emission layer of the light-emitting device may emit blue light.

Another aspect of embodiments of the present disclosure provides an electronic apparatus including the light-emitting device.

In an embodiment, the electron apparatus may further include a thin-film transistor,
the thin-film transistor may include a source electrode and a drain electrode, and
the first electrode of the light-emitting device may be electrically connected to at least one of the source electrode or the drain electrode of the thin-film transistor.

In an embodiment, the electronic apparatus may further include a color filter, a color conversion layer, a touch screen layer, a polarizing layer, or any combination thereof.

The term “interlayer,” as used herein, refers to a single layer and/or all layers between a first electrode and a second electrode of a light-emitting device. A material included in the “interlayer” may be an organic material, an inorganic material, or any combination thereof.

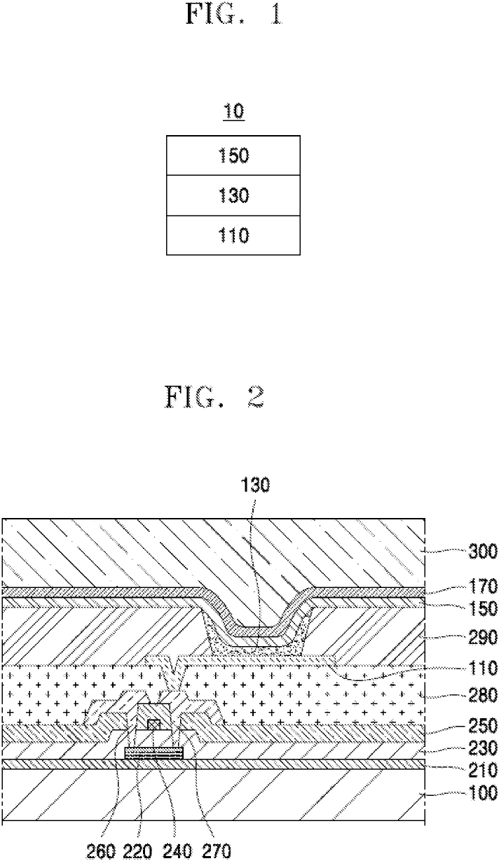

### Description of FIG. 1

FIG. 1 is a schematic cross-sectional view of a light-emitting device 10 according to an embodiment. The light-emitting device 10 may include a first electrode 110, an interlayer 130, and a second electrode 150.

Hereinafter, the structure of the light-emitting device 10 according to an embodiment and a method of manufacturing the light-emitting device 10 will be described with reference to FIG. 1.

### First Electrode 110

In FIG. 1, a substrate may be additionally under the first electrode 110 and/or on the second electrode 150. As the substrate, a glass substrate and/or a plastic substrate may be used. In one or more embodiments, the substrate may be a flexible substrate, and may include plastics having excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing and/or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure consisting of a single layer or a multi-layered structure including a plurality of layers. For example, the first electrode 110 may have a three-layered structure of ITO/Ag/ITO.

### Interlayer 130

The interlayer 130 may be on the first electrode 110. The interlayer 130 may include an emission layer.

The interlayer 130 may further include a hole transport region between the first electrode 110 and the emission layer and an electron transport region between the emission layer and the second electrode 150.

The interlayer 130 may further include, in addition to various suitable organic materials, a metal-containing compound (e.g., an organometallic compound), an inorganic material (e.g., a quantum dot), and/or the like.

In one or more embodiments, the interlayer 130 may include, i) two or more emitting units sequentially stacked between the first electrode 110 and the second electrode 150, and ii) a charge generation layer between the two or more emitting units. When the interlayer 130 includes the emitting units and the charge generation layer as described above, the light-emitting device 10 may be a tandem light-emitting device.

### Hole Transport Region in Interlayer 130

The hole transport region may have i) a single-layered structure consisting of a single layer consisting of a single material, ii) a single-layered structure consisting of a single layer consisting of a plurality of different materials, or iii) a multi-layered structure including a plurality of layers including different materials.

The hole transport region may include a hole transport layer, a hole injection layer, an emission auxiliary layer, an electron blocking layer, or any combination thereof.

For example, the hole transport region may have a multi-layered structure including a hole injection layer/hole transport layer structure, a hole injection layer/hole transport layer/emission auxiliary layer structure, a hole transport layer/emission auxiliary layer structure, or a hole injection layer/hole transport layer/electron blocking layer structure, the layers of each structure being stacked sequentially from the first electrode 110, but embodiments are not limited thereto.

The hole transport layer may include the compound represented by Formula 1 according to an embodiment.

The hole transport region may include a compound represented by Formula 201, a compound represented by Formula 202, or any combination thereof (wherein the hole transport layer is excluded):

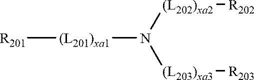

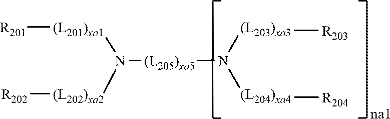

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*’, *—S—*’, *-N(Q₂₀₁)-*’, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ (wherein spirobifluorene is excluded from L₂₀₅),
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be bonded to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group and/or the like) unsubstituted or substituted with at least one R₁₀ₐ (for example, Compound HT16),
R₂₀₃ and R₂₀₄ may optionally be bonded to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

For example, each of Formulae 201 and 202 may include at least one selected from groups represented by Formulae CY201 to CY217:

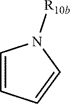

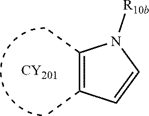

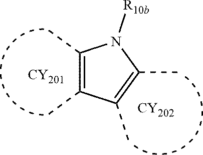

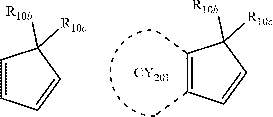

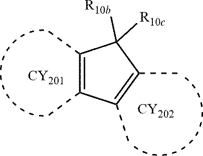

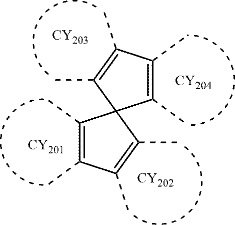

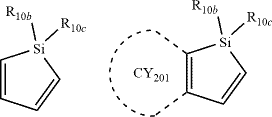

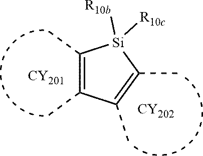

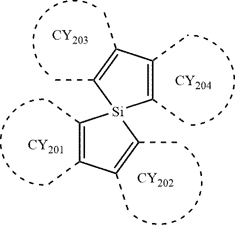

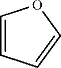

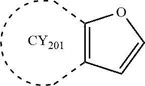

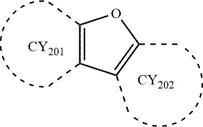

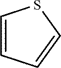

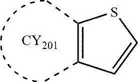

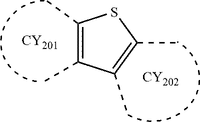

wherein, in Formulae CY201 to CY217, R_{10b} and R_{10c} are the same as described in connection with R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In an embodiment, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one selected from groups represented by Formulae CY201 to CY203.

In one or more embodiments, Formula 201 may include at least one selected from groups represented by Formulae CY201 to CY203 and at least one selected from groups represented by Formulae CY204 to CY217.

In one or more embodiments, in Formula 201, xa1 may be 1, R₂₀₁ may be a group represented by one selected from Formulae CY201 to CY203, xa2 may be 0, and R₂₀₂ may be a group represented by one selected from Formulae CY204 to CY207.

In one or more embodiments, each of Formulae 201 and 202 may not include a group represented by one selected from Formulae CY201 to CY203.

In one or more embodiments, each of Formulae 201 and 202 may not include a group represented by one selected from Formulae CY201 to CY203, and may include at least one selected from groups represented by Formulae CY204 to CY217.

In one or more embodiments, each of Formulae 201 and 202 may not include a group represented by one selected from Formulae CY201 to CY217.

For example, the hole transport region may include one selected from Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

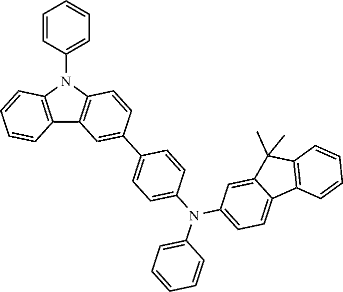 [SMILES: CC1(C)c2ccccc2-c2ccc(N(c3ccccc3)c3ccc(-c4ccc5c(c4)c4ccccc4n5-c4ccccc4)cc3)cc21]

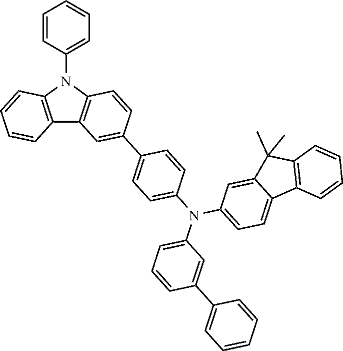 [SMILES: CC1(C)c2ccccc2-c2ccc(N(c3ccc(-c4ccc5c(c4)c4ccccc4n5-c4ccccc4)cc3)c3cccc(-c4ccccc4)c3)cc21]

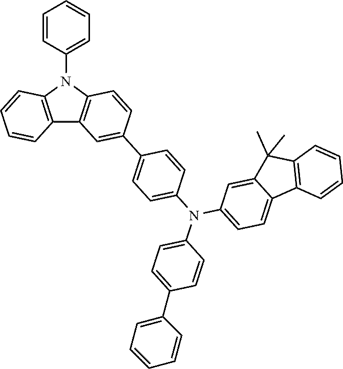 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

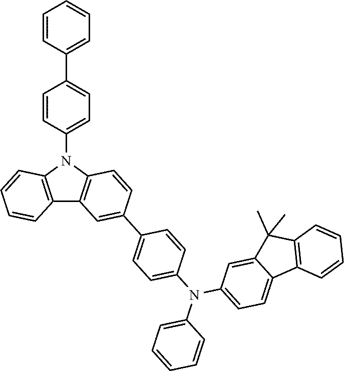 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21]

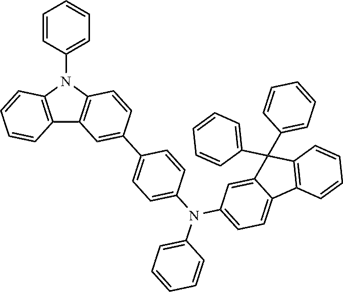 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1]

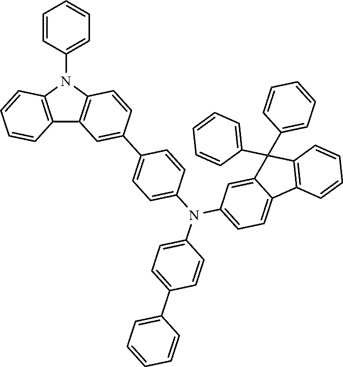 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

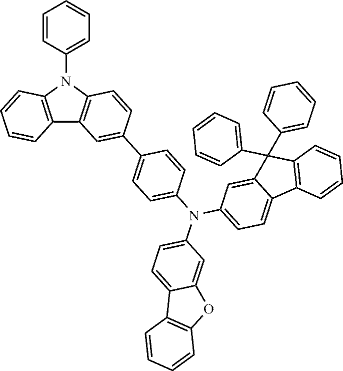 [SMILES: c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1]

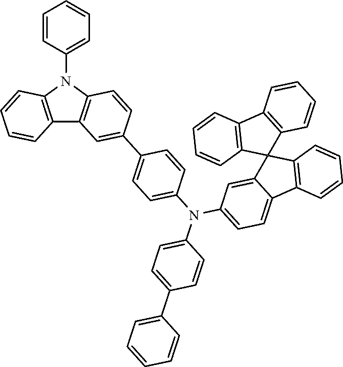 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

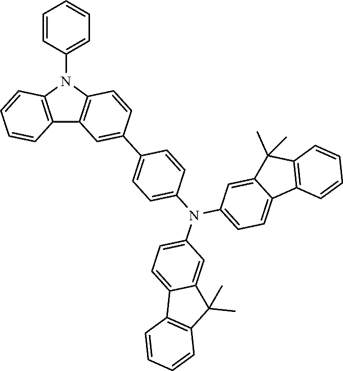 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

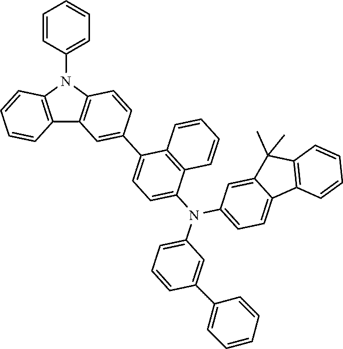 [SMILES: CC1(C)c2ccccc2-c2ccc(N(c3cccc(-c4ccccc4)c3)c3ccc(-c4ccc5c(c4)c4ccccc4n5-c4ccccc4)c4ccccc34)cc21]

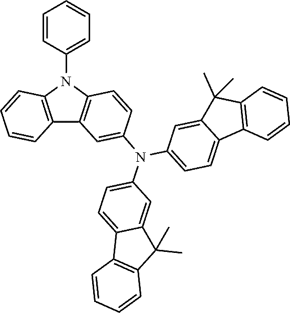 [SMILES: CC1(C)c2ccccc2-c2ccc(N(c3ccc4c(c3)C(C)(C)c3ccccc3-4)c3ccc4c(c3)c3ccccc3n4-c3ccccc3)cc21]

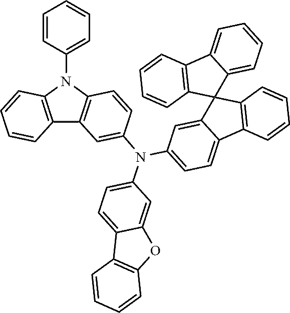 [SMILES: c1ccc(-n2c3ccccc3c3cc(N(c4ccc5c(c4)C4(c6ccccc6-c6ccccc64)c4ccccc4-5)c4ccc5c(c4)oc4ccccc45)ccc32)cc1]

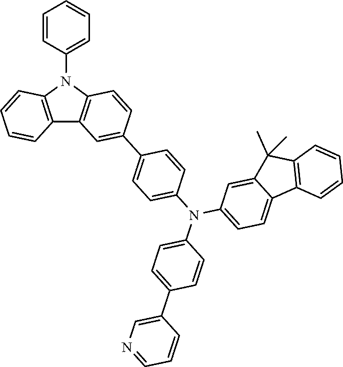 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21]

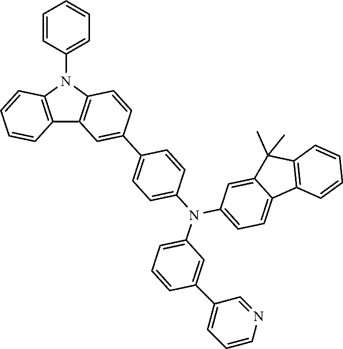 [SMILES: CC1(C)c2ccccc2-c2ccc(N(c3ccc(-c4ccc5c(c4)c4ccccc4n5-c4ccccc4)cc3)c3cccc(-c4cccnc4)c3)cc21]

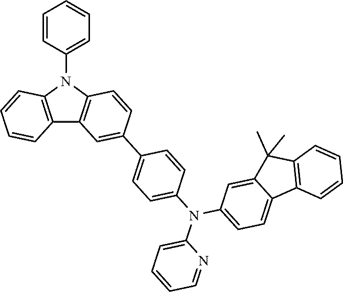 [SMILES: CC1(C)c2ccccc2-c2ccc(N(c3ccc(-c4ccc5c(c4)c4ccccc4n5-c4ccccc4)cc3)c3ccccn3)cc21]

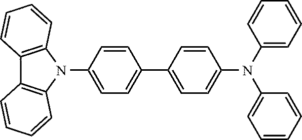 [SMILES: c1ccc(N(c2ccccc2)c2ccc(-c3ccc(-n4c5ccccc5c5ccccc54)cc3)cc2)cc1]

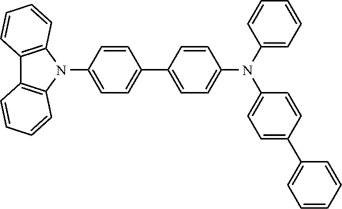 [SMILES: c1ccc(-c2ccc(N(c3ccccc3)c3ccc(-c4ccc(-n5c6ccccc6c6ccccc65)cc4)cc3)cc2)cc1]

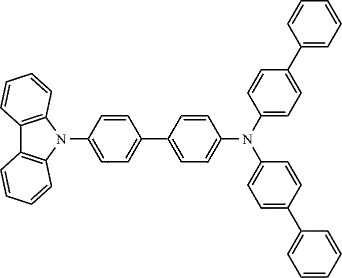 [SMILES: c1ccc(-c2ccc(N(c3ccc(-c4ccccc4)cc3)c3ccc(-c4ccc(-n5c6ccccc6c6ccccc65)cc4)cc3)cc2)cc1]

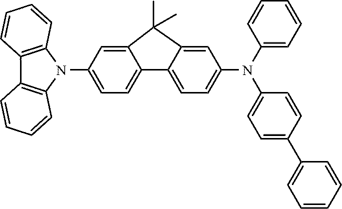 [SMILES: CC1(C)c2cc(N(c3ccccc3)c3ccc(-c4ccccc4)cc3)ccc2-c2ccc(-n3c4ccccc4c4ccccc43)cc21]

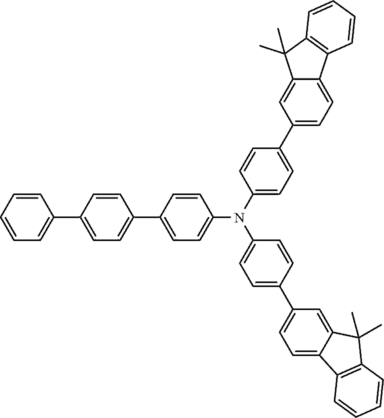 [SMILES: CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

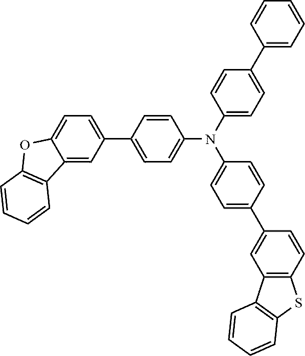 [SMILES: c1ccc(-c2ccc(N(c3ccc(-c4ccc5oc6ccccc6c5c4)cc3)c3ccc(-c4ccc5sc6ccccc6c5c4)cc3)cc2)cc1]

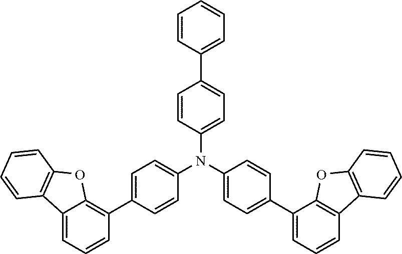 [SMILES: c1ccc(-c2ccc(N(c3ccc(-c4cccc5c4oc4ccccc45)cc3)c3ccc(-c4cccc5c4oc4ccccc45)cc3)cc2)cc1]

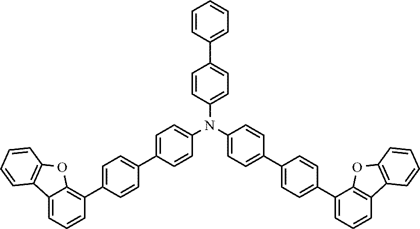 [SMILES: c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

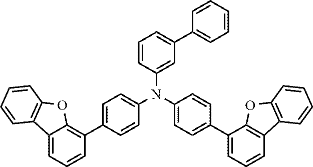 [SMILES: c1ccc(-c2cccc(N(c3ccc(-c4cccc5c4oc4ccccc45)cc3)c3ccc(-c4cccc5c4oc4ccccc45)cc3)c2)cc1]

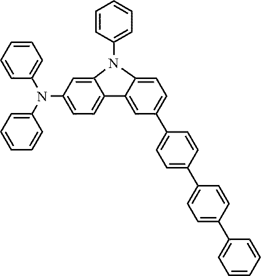 [SMILES: c1ccc(-c2ccc(-c3ccc(-c4ccc5c(c4)c4ccc(N(c6ccccc6)c6ccccc6)cc4n5-c4ccccc4)cc3)cc2)cc1]

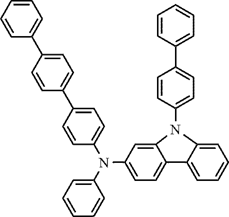 [SMILES: c1ccc(-c2ccc(-c3ccc(N(c4ccccc4)c4ccc5c6ccccc6n(-c6ccc(-c7ccccc7)cc6)c5c4)cc3)cc2)cc1]

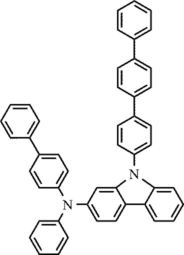 [SMILES: c1ccc(-c2ccc(-c3ccc(-n4c5ccccc5c5ccc(N(c6ccccc6)c6ccc(-c7ccccc7)cc6)cc54)cc3)cc2)cc1]

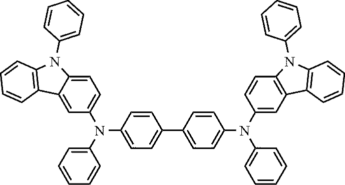 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

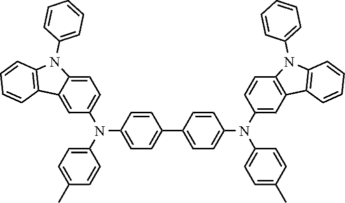 [SMILES: Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1]

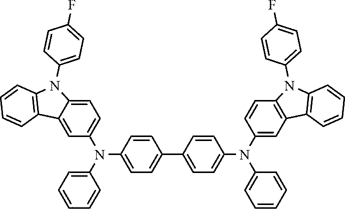 [SMILES: Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

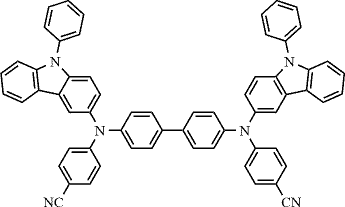

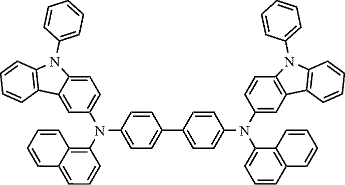 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1]

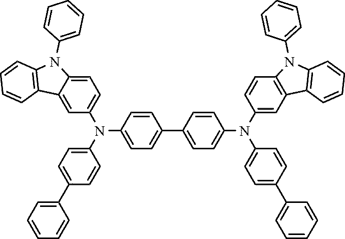 [SMILES: c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1]

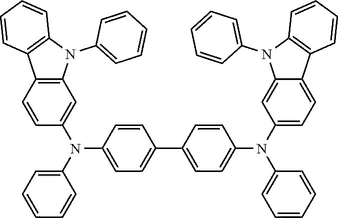 [SMILES: c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1]

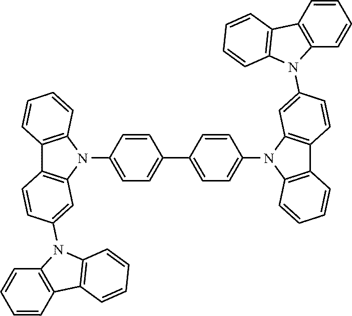 [SMILES: c1ccc2c(c1)c1ccccc1n2-c1ccc2c3ccccc3n(-c3ccc(-c4ccc(-n5c6ccccc6c6ccc(-n7c8ccccc8c8ccccc87)cc65)cc4)cc3)c2c1]

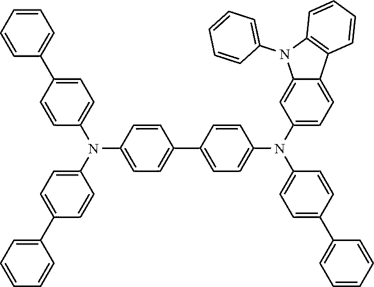 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1]

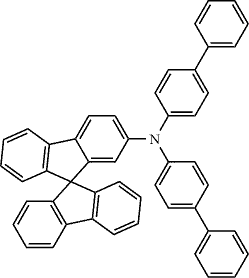 [SMILES: c1ccc(-c2ccc(N(c3ccc(-c4ccccc4)cc3)c3ccc4c(c3)C3(c5ccccc5-c5ccccc53)c3ccccc3-4)cc2)cc1]

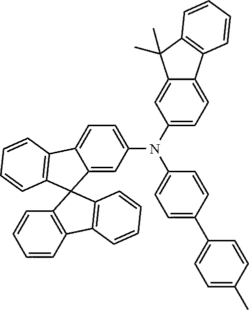 [SMILES: Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1]

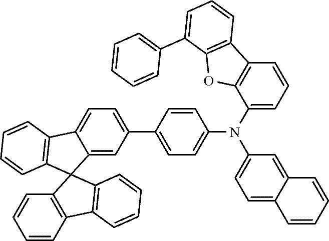 [SMILES: c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

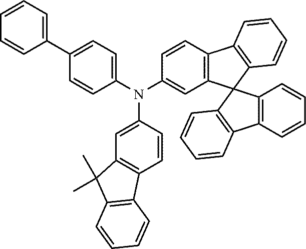 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21]

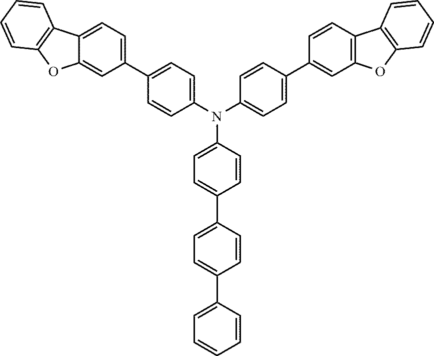 [SMILES: c1ccc(-c2ccc(-c3ccc(N(c4ccc(-c5ccc6c(c5)oc5ccccc56)cc4)c4ccc(-c5ccc6c(c5)oc5ccccc56)cc4)cc3)cc2)cc1]

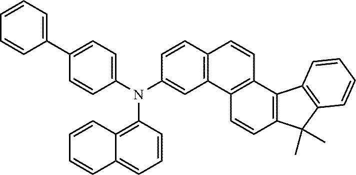 [SMILES: CC1(C)c2ccccc2-c2c1ccc1c2ccc2ccc(N(c3ccc(-c4ccccc4)cc3)c3cccc4ccccc34)cc21]

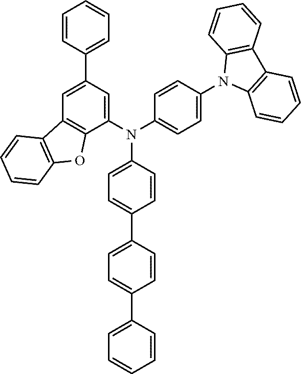 [SMILES: c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

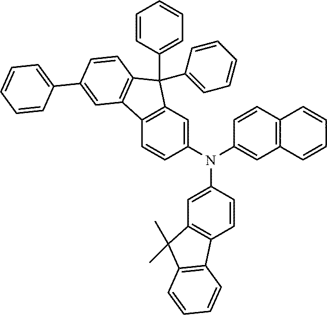 [SMILES: CC1(C)c2ccccc2-c2ccc(N(c3ccc4c(c3)C(c3ccccc3)(c3ccccc3)c3ccc(-c5ccccc5)cc3-4)c3ccc4ccccc4c3)cc21]

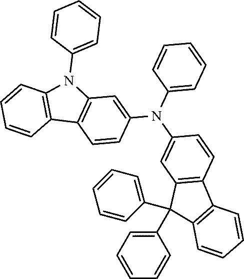 [SMILES: c1ccc(N(c2ccc3c(c2)C(c2ccccc2)(c2ccccc2)c2ccccc2-3)c2ccc3c4ccccc4n(-c4ccccc4)c3c2)cc1]

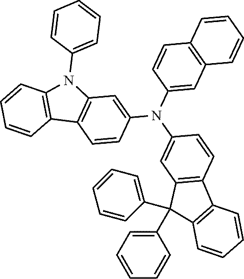 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

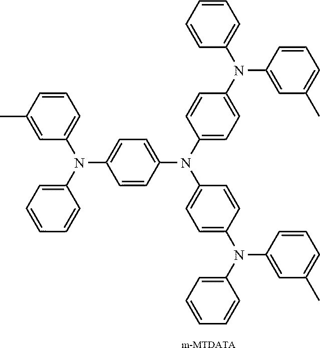

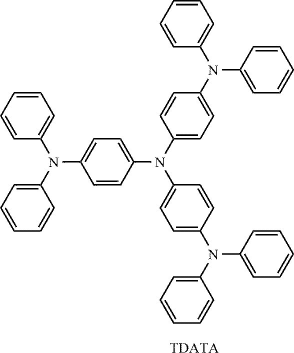

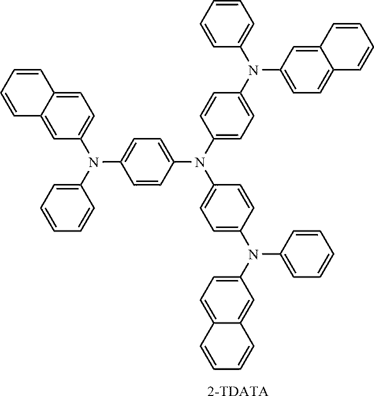

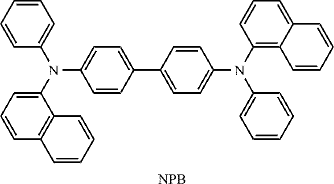 [SMILES: c1ccc(N(c2ccc(-c3ccc(N(c4ccccc4)c4cccc5ccccc45)cc3)cc2)c2cccc3ccccc23)cc1 ; "NPB"]

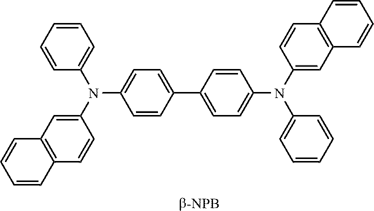 [SMILES: c1ccc(N(c2ccc(-c3ccc(N(c4ccccc4)c4ccc5ccccc5c4)cc3)cc2)c2ccc3ccccc3c2)cc1 ; "b-NPB"]

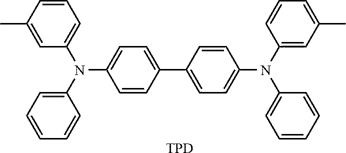

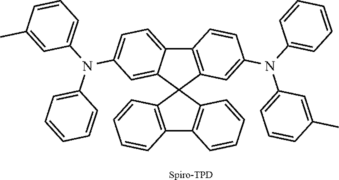

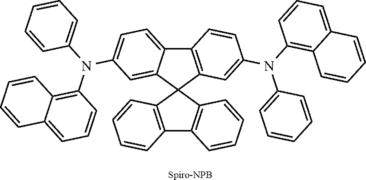

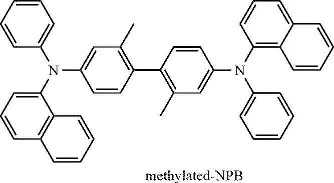 [SMILES: Cc1cc(N(c2ccccc2)c2cccc3ccccc23)ccc1-c1ccc(N(c2ccccc2)c2cccc3ccccc23)cc1C ; "methylated-NPB"]

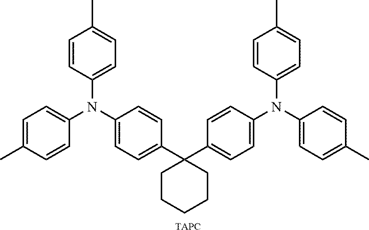

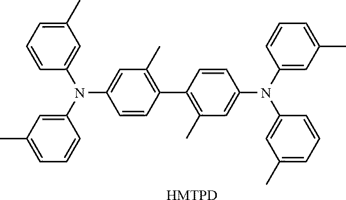

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within the ranges described above, suitable or satisfactory hole transporting characteristics may be obtained without a substantial increase in driving voltage.

The emission auxiliary layer may increase light-emission efficiency by compensating for an optical resonance distance according to the wavelength of light emitted from the emission layer, and the electron blocking layer may block or reduce the leakage of electrons from the emission layer to the hole transport region. Materials that may be included in the hole transport region may be included in the emission auxiliary layer and the electron blocking layer.

### P-Dopant

The hole transport region may further include, in addition to the materials as described above, a charge-generation material for improving conductive properties (e.g., electrically conductive properties). The charge-generation material may be uniformly or non-uniformly dispersed in the hole transport region (for example, in the form of a single layer consisting of a charge-generation material).

The charge-generation material may be, for example, a p-dopant.

For example, a lowest unoccupied molecular orbital (LUMO) energy level of the p-dopant may be -3.5 eV or less.

In an embodiment, the p-dopant may include a quinone derivative, a cyano group-containing compound, a compound containing element EL1 and element EL2, or any combination thereof.

Examples of the quinone derivative may include TCNQ, F4-TCNQ, and the like.

Examples of the cyano group-containing compound may include HAT-CN, a compound represented by Formula 221, and the like:

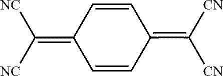

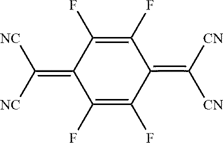

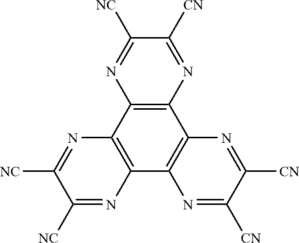

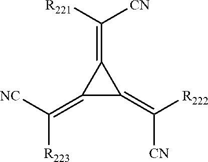

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one selected from R₂₂₁ to R₂₂₃ may each independently be a C₃-C₆₀ carbocyclic group or a C₁-C₆₀ heterocyclic group, each substituted with a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be a metal, a metalloid, or any combination thereof, and element EL2 may be a non-metal, a metalloid, or any combination thereof.

Examples of the metal may include: an alkali metal (for example, lithium (Li), sodium (Na), potassium (K), rubidium (Rb), cesium (Cs), and/or the like); an alkaline earth metal (for example, beryllium (Be), magnesium (Mg), calcium (Ca), strontium (Sr), barium (Ba), and/or the like); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (lr), nickel (Ni), palladium (Pd), platinum (Pt), copper (Cu), silver (Ag), gold (Au), or the like); a post-transition metal (for example, zinc (Zn), indium (In), tin (Sn), and/or the like); a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like); and the like.

Examples of the metalloid may include silicon (Si), antimony (Sb), tellurium (Te), and the like.

Examples of the non-metal may include oxygen (O), halogen (for example, F, Cl, Br, I, and the like), and the like.

For example, the compound containing element EL1 and element EL2 may include metal oxide, metal halide (for example, metal fluoride, metal chloride, metal bromide, metal iodide, and the like), metalloid halide (for example, metalloid fluoride, metalloid chloride, metalloid bromide, metalloid iodide, and the like), metal telluride, or any combination thereof.

Examples of the metal oxide may include tungsten oxide (for example, WO, W₂O₃, WO₂, WO₃, W₂O₅, and the like), vanadium oxide (for example, VO, V₂O₃, VO₂, V₂O₅, and the like), molybdenum oxide (MoO, Mo₂O₃, MoO₂, MoO₃, Mo₂O₅, and the like), rhenium oxide (for example, ReOs and the like), and the like.

Examples of the metal halide may include alkali metal halide, alkaline earth metal halide, transition metal halide, post-transition metal halide, lanthanide metal halide, and the like.

Examples of the alkali metal halide may include LiF, NaF, KF, RbF, CsF, LiCI, NaCl, KCI, RbCI, CsCI, LiBr, NaBr, KBr, RbBr, CsBr, Lil, Nal, Kl, Rbl, Csl, and the like.

Examples of the alkaline earth metal halide may include BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, Bel₂, Mgl₂, Cal₂, Srl₂, Bal₂, and the like.

Examples of the transition metal halide may include titanium halide (for example, TiF₄, TiCl₄, TiBr₄, Til₄, and the like), zirconium halide (for example, ZrF₄, ZrCl₄, ZrBr₄, Zrl₄, and the like), hafnium halide (for example HfF₄, HfCl₄, HfBr₄, Hfl₄, and the like), vanadium halide (for example, VF₃, VCI₃, VBr₃, Vl₃, and the like), niobium halide (for example, NbF₃, NbCl₃, NbBr₃, Nbl₃, and the like), tantalum halide (for example, TaF₃, TaCl₃, TaBr₃, Tal3, and the like), chromium halide (for example, CrF₃, CrCl₃, CrBr₃, Crl3, and the like), molybdenum halide (for example, MoF₃, MoCl₃, MoBr₃, Mol₃, and the like), tungsten halide (for example, WF₃, WCl₃, WBr₃, Wl₃, and the like), manganese halide (for example, MnF₂, MnCl₂, MnBr₂, Mnl₂, and the like), technetium halide (for example, TcF₂, TcCl₂, TcBr₂, Tcl₂, and the like), rhenium halide (for example, ReF₂, ReCl₂, ReBr₂, Rel₂, and the like), iron halide (for example, FeF₂, FeCl₂, FeBr₂, Fel₂, and the like), ruthenium halide (for example, RuF₂, RuCl₂, RuBr₂, Rul₂, and the like), osmium halide (for example, OsF₂, OsCl₂, OsBr₂, Osl₂, and the like), cobalt halide (for example, CoF₂, CoCl₂, CoBr₂, Col₂, and the like), rhodium halide (for example, RhF₂, RhCl₂, RhBr₂, Rhl₂, and the like), iridium halide (for example, IrF₂, IrCl₂, IrBr₂, Irl₂, and the like), nickel halide (for example, NiF₂, NiCl₂, NiBr₂, Nil₂, and the like), palladium halide (for example, PdF₂, PdCl₂, PdBr₂, Pdl₂, and the like), platinum halide (for example, PtF₂, PtCl₂, PtBr₂, Ptl₂, and the like), copper halide (for example, CuF, CuCI, CuBr, Cul, and the like), silver halide (for example, AgF, AgCl, AgBr, Agl, and the like), gold halide (for example, AuF, AuCI, AuBr, Aul, and the like), and the like.

Examples of the post-transition metal halide may include zinc halide (for example, ZnF₂, ZnCl₂, ZnBr₂, Znl₂, and the like), indium halide (for example, lnl₃ and the like), tin halide (for example, Snl₂ and the like), and the like.

Examples of the lanthanide metal halide may include YbF, YbF₂, YbF₃, SmF₃, YbCl, YbCl₂, YbCl₃, SmCl₃, YbBr, YbBr₂, YbBr₃, SmBr₃, Ybl, Ybl₂, Ybl₃, Sml₃, and the like.

Examples of the metalloid halide may include antimony halide (for example, SbCl₅ and the like) and the like.

Examples of the metal telluride may include alkali metal telluride (for example, Li₂Te, Na₂Te, K₂Te, Rb₂Te, Cs₂Te, and the like), alkaline earth metal telluride (for example, BeTe, MgTe, CaTe, SrTe, BaTe, and the like), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and the like), post-transition metal telluride (for example, ZnTe and the like), lanthanide metal telluride (for example, LaTe, CeTe, PrTe, NdTe, PmTe, EuTe, GdTe, TbTe, DyTe, HoTe, ErTe, TmTe, YbTe, LuTe, and the like), and the like.

### Emission Layer in Interlayer 130

When the light-emitting device 10 is a full-color light-emitting device, the emission layer may be patterned into a red emission layer, a green emission layer, and/or a blue emission layer, according to a subpixel. In one or more embodiments, the emission layer may have a stacked structure of two or more layers of a red emission layer, a green emission layer, and a blue emission layer, in which the two or more layers contact (e.g., physically contact) each other or are spaced apart from each other to emit white light. In one or more embodiments, the emission layer may have a structure in which two or more materials of a red light-emitting material, a green light-emitting material, and a blue light-emitting material are mixed together with each other in a single layer, and thus emit white light.

The emission layer may include a host and a dopant. The dopant may include a phosphorescent dopant, a fluorescent dopant, or any combination thereof.

An amount of the dopant in the emission layer may be from about 0.01 part by weight to about 15 parts by weight based on 100 parts by weight of the host, but embodiments are not limited thereto.

In one or more embodiments, the emission layer may include a quantum dot.

In one or more embodiments, the emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within the ranges described above, excellent light-emission characteristics may be obtained without a substantial increase in driving voltage.

### Host

The host may include a compound represented by Formula 301:

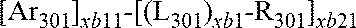

wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ,—Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ are each the same as described in connection with Q₁.

For example, when xb11 in Formula 301 is 2 or more, two or more of Ar₃₀₁(s) may be bonded to each other via a single bond.

In one or more embodiments, the host may include a compound represented by Formula 301-1, a compound represented by Formula 301-2, or any combination thereof:

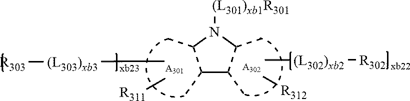

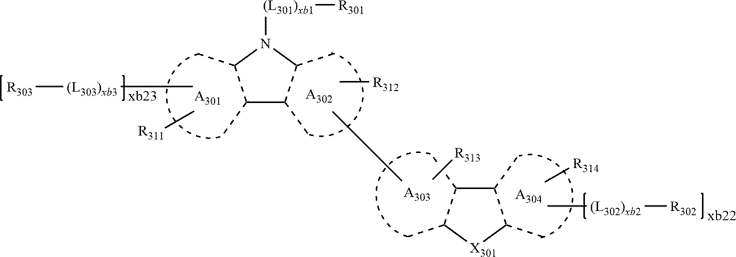

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ are respectively the same as those described in the present specification,
L₃₀₂ to L₃₀₄ are each independently the same as described in connection with L₃₀₁,
xb2 to xb4 are each independently the same as described in connection with xb1, and
R₃₀₂ to R₃₀₅ and R₃₁₁ to R₃₁₄ are each the same as described in connection with R₃₀₁.

In one or more embodiments, the host may include an alkali earth metal complex, a post-transition metal complex, or any combination thereof. For example, the host may include a Be complex (for example, Compound H55), an Mg complex, a Zn complex, or any combination thereof.

In one or more embodiments, the host may include one selected from Compounds H1 to H124, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

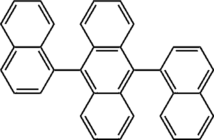 [SMILES: c1ccc2c(-c3c4ccccc4c(-c4cccc5ccccc45)c4ccccc34)cccc2c1]

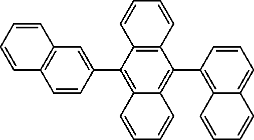 [SMILES: c1ccc2cc(-c3c4ccccc4c(-c4cccc5ccccc45)c4ccccc34)ccc2c1]

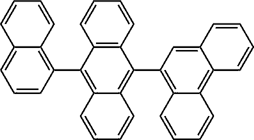 [SMILES: c1ccc2c(-c3c4ccccc4c(-c4cc5ccccc5c5ccccc45)c4ccccc34)cccc2c1]

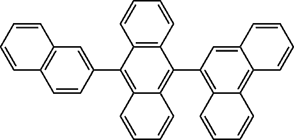 [SMILES: c1ccc2cc(-c3c4ccccc4c(-c4cc5ccccc5c5ccccc45)c4ccccc34)ccc2c1]

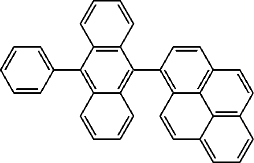 [SMILES: c1ccc(-c2c3ccccc3c(-c3ccc4ccc5cccc6ccc3c4c56)c3ccccc23)cc1]

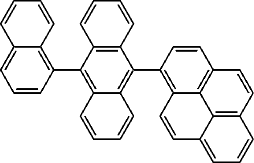 [SMILES: c1ccc2c(-c3c4ccccc4c(-c4ccc5ccc6cccc7ccc4c5c67)c4ccccc34)cccc2c1]

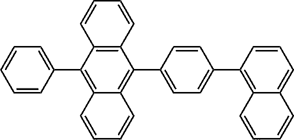 [SMILES: c1ccc(-c2c3ccccc3c(-c3ccc(-c4cccc5ccccc45)cc3)c3ccccc23)cc1]

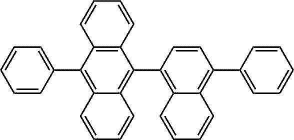 [SMILES: c1ccc(-c2ccc(-c3c4ccccc4c(-c4ccccc4)c4ccccc34)c3ccccc23)cc1]

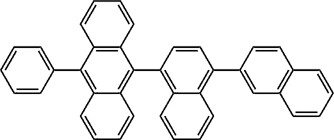 [SMILES: c1ccc(-c2c3ccccc3c(-c3ccc(-c4ccc5ccccc5c4)c4ccccc34)c3ccccc23)cc1]

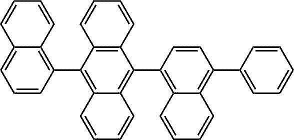 [SMILES: c1ccc(-c2ccc(-c3c4ccccc4c(-c4cccc5ccccc45)c4ccccc34)c3ccccc23)cc1]

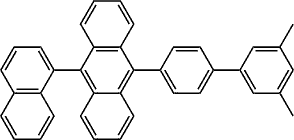 [SMILES: Cc1cc(C)cc(-c2ccc(-c3c4ccccc4c(-c4cccc5ccccc45)c4ccccc34)cc2)c1]

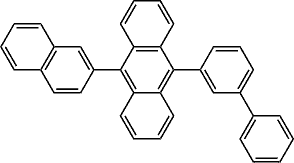 [SMILES: c1ccc(-c2cccc(-c3c4ccccc4c(-c4ccc5ccccc5c4)c4ccccc34)c2)cc1]

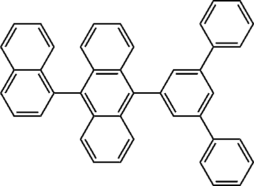 [SMILES: c1ccc(-c2cc(-c3ccccc3)cc(-c3c4ccccc4c(-c4cccc5ccccc45)c4ccccc34)c2)cc1]

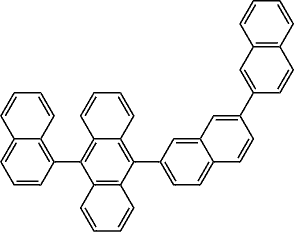 [SMILES: c1ccc2cc(-c3ccc4ccc(-c5c6ccccc6c(-c6cccc7ccccc67)c6ccccc56)cc4c3)ccc2c1]

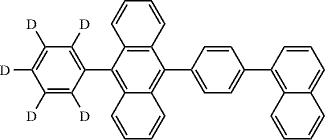 [SMILES: [2H]c1c([2H])c([2H])c(-c2c3ccccc3c(-c3ccc(-c4cccc5ccccc45)cc3)c3ccccc23)c([2H])c1[2H]]

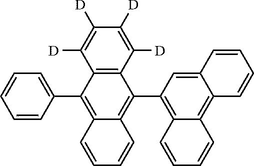 [SMILES: [2H]c1c([2H])c([2H])c2c(-c3cc4ccccc4c4ccccc34)c3ccccc3c(-c3ccccc3)c2c1[2H]]

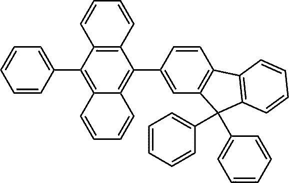 [SMILES: c1ccc(-c2c3ccccc3c(-c3ccc4c(c3)C(c3ccccc3)(c3ccccc3)c3ccccc3-4)c3ccccc23)cc1]

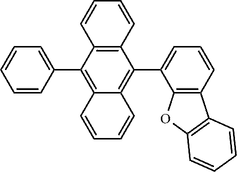 [SMILES: c1ccc(-c2c3ccccc3c(-c3cccc4c3oc3ccccc34)c3ccccc23)cc1]

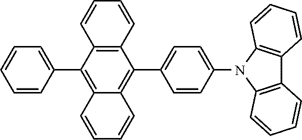 [SMILES: c1ccc(-c2c3ccccc3c(-c3ccc(-n4c5ccccc5c5ccccc54)cc3)c3ccccc23)cc1]

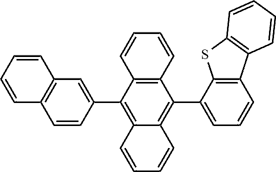 [SMILES: c1ccc2cc(-c3c4ccccc4c(-c4cccc5c4sc4ccccc45)c4ccccc34)ccc2c1]

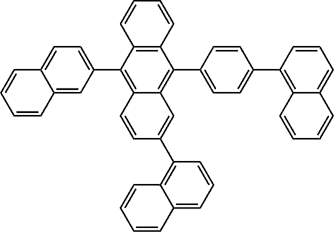 [SMILES: c1ccc2cc(-c3c4ccccc4c(-c4ccc(-c5cccc6ccccc56)cc4)c4cc(-c5cccc6ccccc56)ccc34)ccc2c1]

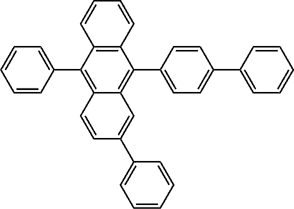 [SMILES: c1ccc(-c2ccc(-c3c4ccccc4c(-c4ccccc4)c4ccc(-c5ccccc5)cc34)cc2)cc1]

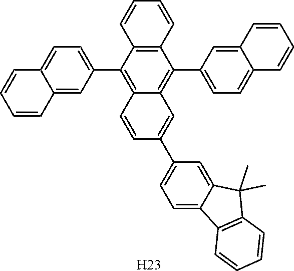 [SMILES: CC1(C)c2ccccc2-c2ccc(-c3ccc4c(-c5ccc6ccccc6c5)c5ccccc5c(-c5ccc6ccccc6c5)c4c3)cc21 ; "H23"]

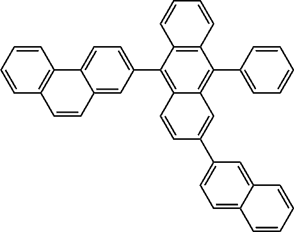 [SMILES: c1ccc(-c2c3ccccc3c(-c3ccc4c(ccc5ccccc54)c3)c3ccc(-c4ccc5ccccc5c4)cc23)cc1]

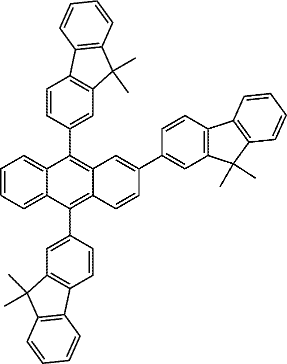 [SMILES: CC1(C)c2ccccc2-c2ccc(-c3ccc4c(-c5ccc6c(c5)C(C)(C)c5ccccc5-6)c5ccccc5c(-c5ccc6c(c5)C(C)(C)c5ccccc5-6)c4c3)cc21]

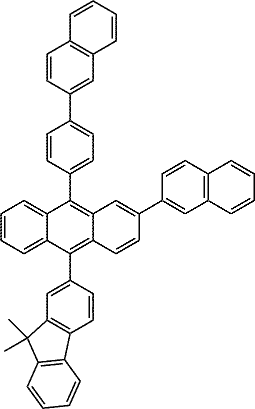 [SMILES: CC1(C)c2ccccc2-c2ccc(-c3c4ccccc4c(-c4ccc(-c5ccc6ccccc6c5)cc4)c4cc(-c5ccc6ccccc6c5)ccc34)cc21]

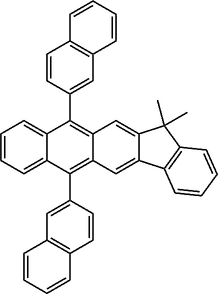 [SMILES: CC1(C)c2ccccc2-c2cc3c(-c4ccc5ccccc5c4)c4ccccc4c(-c4ccc5ccccc5c4)c3cc21]

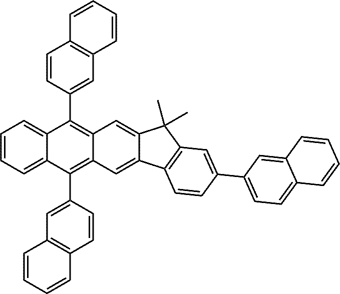 [SMILES: CC1(C)c2cc(-c3ccc4ccccc4c3)ccc2-c2cc3c(-c4ccc5ccccc5c4)c4ccccc4c(-c4ccc5ccccc5c4)c3cc21]

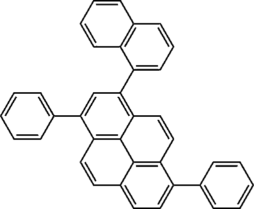 [SMILES: c1ccc(-c2ccc3ccc4c(-c5ccccc5)cc(-c5cccc6ccccc56)c5ccc2c3c45)cc1]

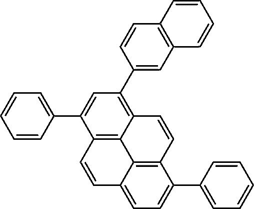 [SMILES: c1ccc(-c2ccc3ccc4c(-c5ccccc5)cc(-c5ccc6ccccc6c5)c5ccc2c3c45)cc1]

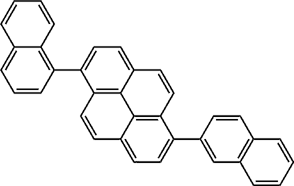 [SMILES: c1ccc2cc(-c3ccc4ccc5c(-c6cccc7ccccc67)ccc6ccc3c4c65)ccc2c1]

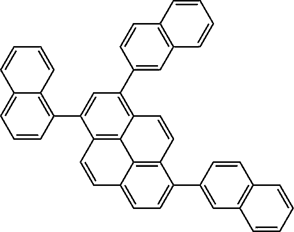 [SMILES: c1ccc2cc(-c3ccc4ccc5c(-c6cccc7ccccc67)cc(-c6ccc7ccccc7c6)c6ccc3c4c65)ccc2c1]

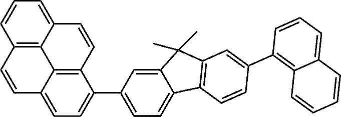 [SMILES: CC1(C)c2cc(-c3cccc4ccccc34)ccc2-c2ccc(-c3ccc4ccc5cccc6ccc3c4c56)cc21]

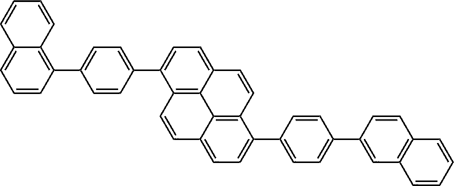 [SMILES: c1ccc2cc(-c3ccc(-c4ccc5ccc6c(-c7ccc(-c8cccc9ccccc89)cc7)ccc7ccc4c5c76)cc3)ccc2c1]

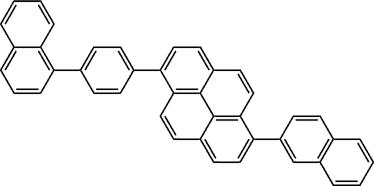 [SMILES: c1ccc2cc(-c3ccc4ccc5c(-c6ccc(-c7cccc8ccccc78)cc6)ccc6ccc3c4c65)ccc2c1]

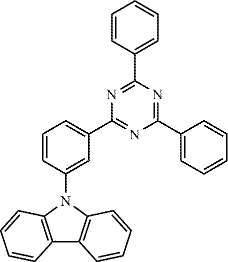 [SMILES: c1ccc(-c2nc(-c3ccccc3)nc(-c3cccc(-n4c5ccccc5c5ccccc54)c3)n2)cc1]

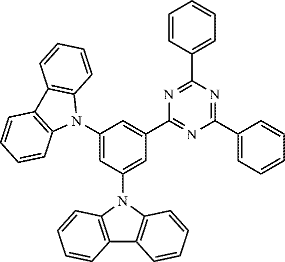 [SMILES: c1ccc(-c2nc(-c3ccccc3)nc(-c3cc(-n4c5ccccc5c5ccccc54)cc(-n4c5ccccc5c5ccccc54)c3)n2)cc1]

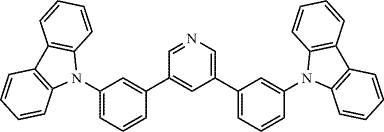 [SMILES: c1cc(-c2cncc(-c3cccc(-n4c5ccccc5c5ccccc54)c3)c2)cc(-n2c3ccccc3c3ccccc32)c1]

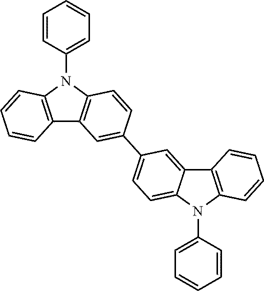 [SMILES: c1ccc(-n2c3ccccc3c3cc(-c4ccc5c(c4)c4ccccc4n5-c4ccccc4)ccc32)cc1]

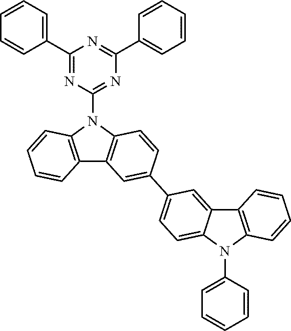 [SMILES: c1ccc(-c2nc(-c3ccccc3)nc(-n3c4ccccc4c4cc(-c5ccc6c(c5)c5ccccc5n6-c5ccccc5)ccc43)n2)cc1]

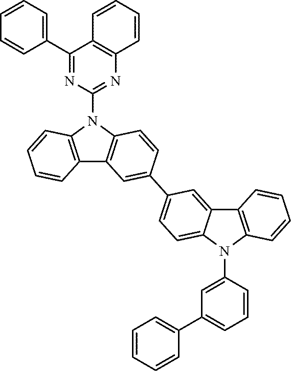 [SMILES: c1ccc(-c2cccc(-n3c4ccccc4c4cc(-c5ccc6c(c5)c5ccccc5n6-c5nc(-c6ccccc6)c6ccccc6n5)ccc43)c2)cc1]

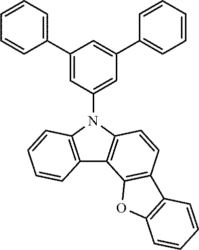 [SMILES: c1ccc(-c2cc(-c3ccccc3)cc(-n3c4ccccc4c4c5oc6ccccc6c5ccc43)c2)cc1]

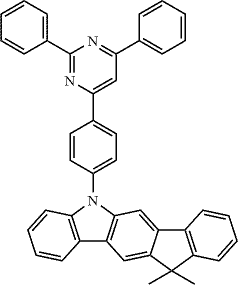 [SMILES: CC1(C)c2ccccc2-c2cc3c(cc21)c1ccccc1n3-c1ccc(-c2cc(-c3ccccc3)nc(-c3ccccc3)n2)cc1]

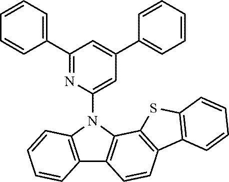 [SMILES: c1ccc(-c2cc(-c3ccccc3)nc(-n3c4ccccc4c4ccc5c6ccccc6sc5c43)c2)cc1]

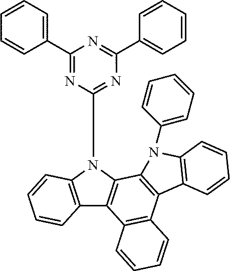 [SMILES: c1ccc(-c2nc(-c3ccccc3)nc(-n3c4ccccc4c4c5ccccc5c5c6ccccc6n(-c6ccccc6)c5c43)n2)cc1]

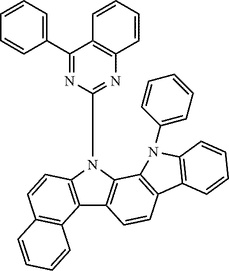 [SMILES: c1ccc(-c2nc(-n3c4ccc5ccccc5c4c4ccc5c6ccccc6n(-c6ccccc6)c5c43)nc3ccccc23)cc1]

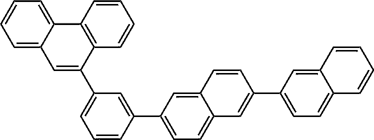 [SMILES: c1cc(-c2ccc3cc(-c4ccc5ccccc5c4)ccc3c2)cc(-c2cc3ccccc3c3ccccc23)c1]

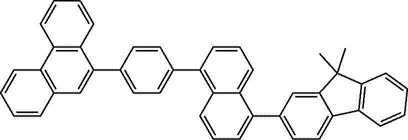 [SMILES: CC1(C)c2ccccc2-c2ccc(-c3cccc4c(-c5ccc(-c6cc7ccccc7c7ccccc67)cc5)cccc34)cc21]

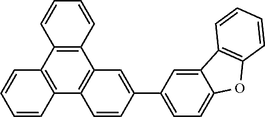 [SMILES: c1ccc2c(c1)oc1ccc(-c3ccc4c5ccccc5c5ccccc5c4c3)cc12]

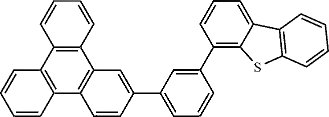 [SMILES: c1cc(-c2ccc3c4ccccc4c4ccccc4c3c2)cc(-c2cccc3c2sc2ccccc23)c1]

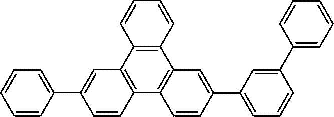 [SMILES: c1ccc(-c2cccc(-c3ccc4c5ccc(-c6ccccc6)cc5c5ccccc5c4c3)c2)cc1]

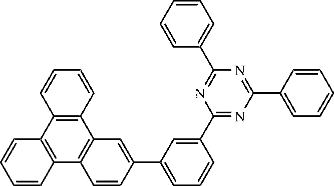 [SMILES: c1ccc(-c2nc(-c3ccccc3)nc(-c3cccc(-c4ccc5c6ccccc6c6ccccc6c5c4)c3)n2)cc1]

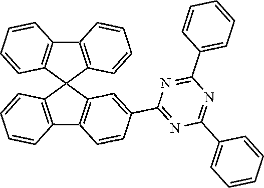 [SMILES: c1ccc(-c2nc(-c3ccccc3)nc(-c3ccc4c(c3)C3(c5ccccc5-c5ccccc53)c3ccccc3-4)n2)cc1]

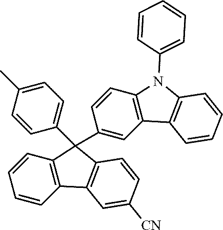 [SMILES: Cc1ccc(C2(c3ccc4c(c3)c3ccccc3n4-c3ccccc3)c3ccccc3-c3cc(C#N)ccc32)cc1]

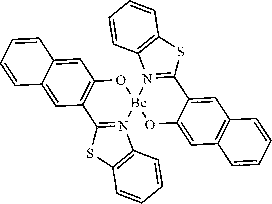

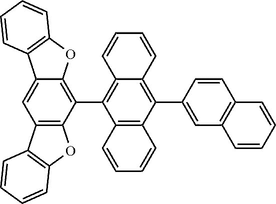 [SMILES: c1ccc2cc(-c3c4ccccc4c(-c4c5oc6ccccc6c5cc5c4oc4ccccc45)c4ccccc34)ccc2c1]

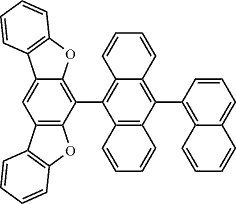 [SMILES: c1ccc2c(-c3c4ccccc4c(-c4c5oc6ccccc6c5cc5c4oc4ccccc45)c4ccccc34)cccc2c1]

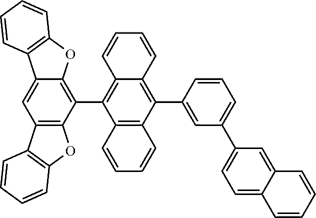 [SMILES: c1cc(-c2ccc3ccccc3c2)cc(-c2c3ccccc3c(-c3c4oc5ccccc5c4cc4c3oc3ccccc34)c3ccccc23)c1]

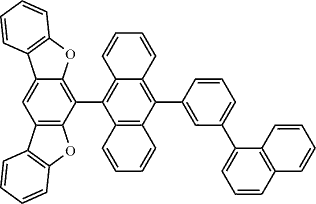 [SMILES: c1cc(-c2cccc3ccccc23)cc(-c2c3ccccc3c(-c3c4oc5ccccc5c4cc4c3oc3ccccc34)c3ccccc23)c1]

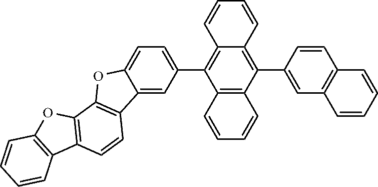 [SMILES: c1ccc2cc(-c3c4ccccc4c(-c4ccc5oc6c(ccc7c8ccccc8oc76)c5c4)c4ccccc34)ccc2c1]

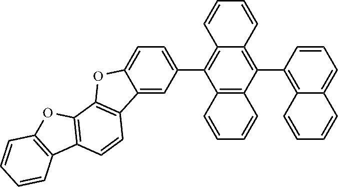 [SMILES: c1ccc2c(-c3c4ccccc4c(-c4ccc5oc6c(ccc7c8ccccc8oc76)c5c4)c4ccccc34)cccc2c1]

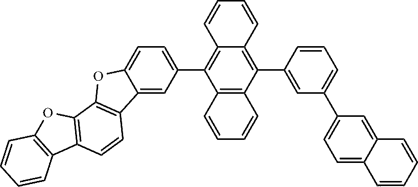 [SMILES: c1cc(-c2ccc3ccccc3c2)cc(-c2c3ccccc3c(-c3ccc4oc5c(ccc6c7ccccc7oc65)c4c3)c3ccccc23)c1]

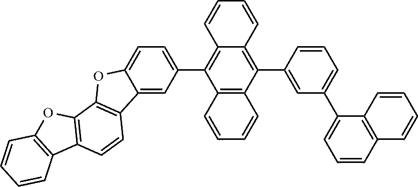 [SMILES: c1cc(-c2cccc3ccccc23)cc(-c2c3ccccc3c(-c3ccc4oc5c(ccc6c7ccccc7oc65)c4c3)c3ccccc23)c1]

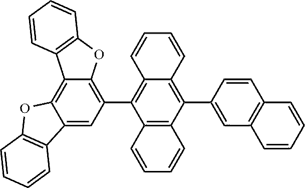 [SMILES: c1ccc2cc(-c3c4ccccc4c(-c4cc5c6ccccc6oc5c5c4oc4ccccc45)c4ccccc34)ccc2c1]

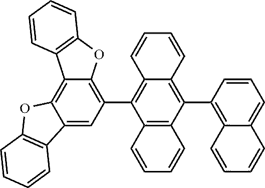 [SMILES: c1ccc2c(-c3c4ccccc4c(-c4cc5c6ccccc6oc5c5c4oc4ccccc45)c4ccccc34)cccc2c1]

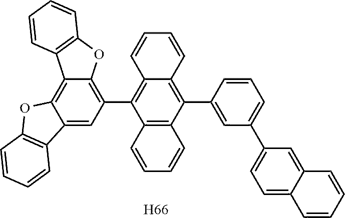 [SMILES: c1cc(-c2ccc3ccccc3c2)cc(-c2c3ccccc3c(-c3cc4c5ccccc5oc4c4c3oc3ccccc34)c3ccccc23)c1 ; "H66"]

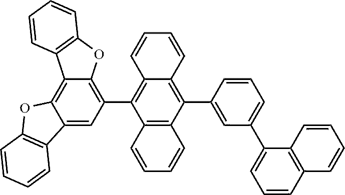 [SMILES: c1cc(-c2cccc3ccccc23)cc(-c2c3ccccc3c(-c3cc4c5ccccc5oc4c4c3oc3ccccc34)c3ccccc23)c1]

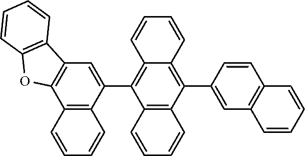 [SMILES: c1ccc2cc(-c3c4ccccc4c(-c4cc5c6ccccc6oc5c5ccccc45)c4ccccc34)ccc2c1]

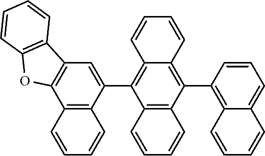 [SMILES: c1ccc2c(-c3c4ccccc4c(-c4cc5c6ccccc6oc5c5ccccc45)c4ccccc34)cccc2c1]

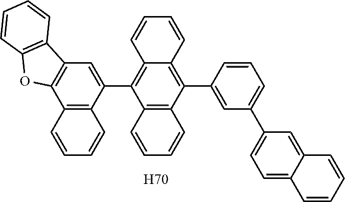

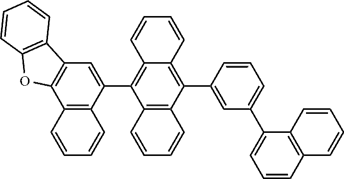 [SMILES: c1cc(-c2cccc3ccccc23)cc(-c2c3ccccc3c(-c3cc4c5ccccc5oc4c4ccccc34)c3ccccc23)c1]

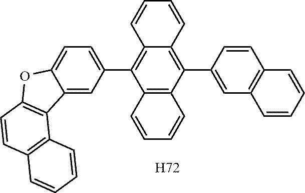

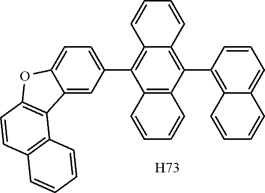

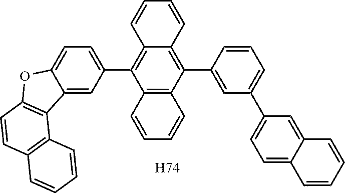

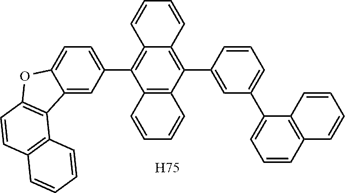

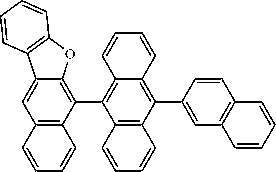 [SMILES: c1ccc2cc(-c3c4ccccc4c(-c4c5ccccc5cc5c4oc4ccccc45)c4ccccc34)ccc2c1]

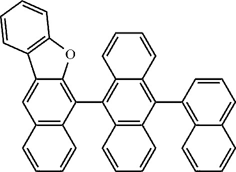 [SMILES: c1ccc2c(-c3c4ccccc4c(-c4c5ccccc5cc5c4oc4ccccc45)c4ccccc34)cccc2c1]

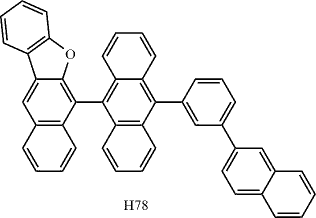

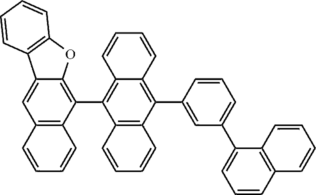 [SMILES: c1cc(-c2cccc3ccccc23)cc(-c2c3ccccc3c(-c3c4ccccc4cc4c3oc3ccccc34)c3ccccc23)c1]

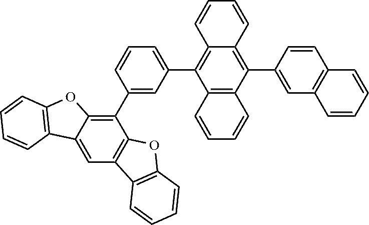 [SMILES: c1cc(-c2c3ccccc3c(-c3ccc4ccccc4c3)c3ccccc23)cc(-c2c3oc4ccccc4c3cc3c2oc2ccccc23)c1]

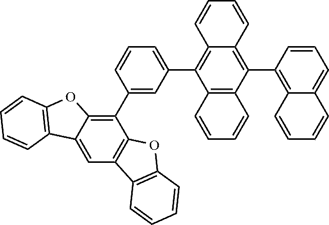 [SMILES: c1cc(-c2c3ccccc3c(-c3cccc4ccccc34)c3ccccc23)cc(-c2c3oc4ccccc4c3cc3c2oc2ccccc23)c1]

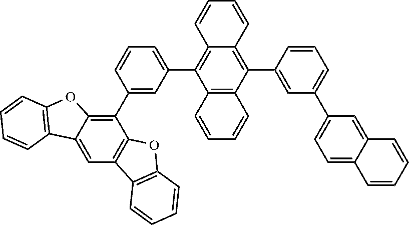 [SMILES: c1cc(-c2ccc3ccccc3c2)cc(-c2c3ccccc3c(-c3cccc(-c4c5oc6ccccc6c5cc5c4oc4ccccc45)c3)c3ccccc23)c1]

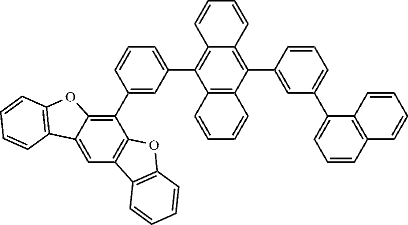 [SMILES: c1cc(-c2cccc3ccccc23)cc(-c2c3ccccc3c(-c3cccc(-c4c5oc6ccccc6c5cc5c4oc4ccccc45)c3)c3ccccc23)c1]

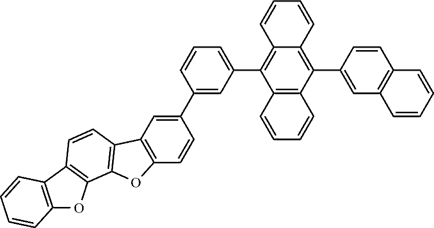 [SMILES: c1cc(-c2ccc3oc4c(ccc5c6ccccc6oc54)c3c2)cc(-c2c3ccccc3c(-c3ccc4ccccc4c3)c3ccccc23)c1]

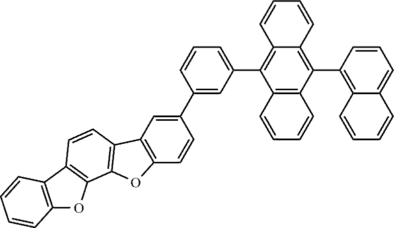 [SMILES: c1cc(-c2ccc3oc4c(ccc5c6ccccc6oc54)c3c2)cc(-c2c3ccccc3c(-c3cccc4ccccc34)c3ccccc23)c1]

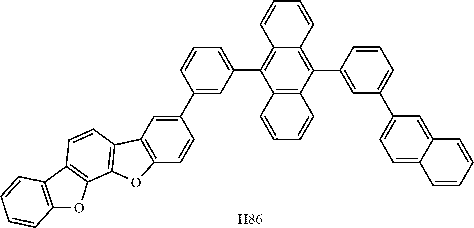 [SMILES: c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; "H86"]

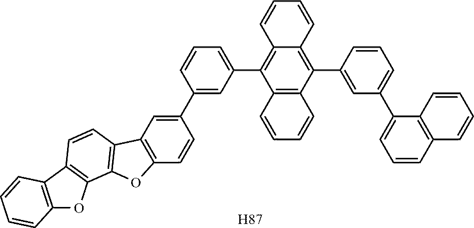 [SMILES: c1cc(-c2ccc3oc4c(ccc5c6ccccc6oc54)c3c2)cc(-c2c3ccccc3c(-c3cccc(-c4cccc5ccccc45)c3)c3ccccc23)c1 ; "H87"]

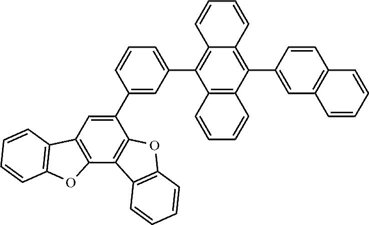 [SMILES: c1cc(-c2c3ccccc3c(-c3ccc4ccccc4c3)c3ccccc23)cc(-c2cc3c4ccccc4oc3c3c2oc2ccccc23)c1]

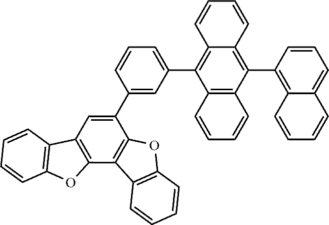 [SMILES: c1cc(-c2c3ccccc3c(-c3cccc4ccccc34)c3ccccc23)cc(-c2cc3c4ccccc4oc3c3c2oc2ccccc23)c1]

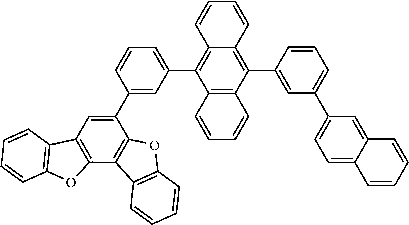 [SMILES: c1cc(-c2ccc3ccccc3c2)cc(-c2c3ccccc3c(-c3cccc(-c4cc5c6ccccc6oc5c5c4oc4ccccc45)c3)c3ccccc23)c1]

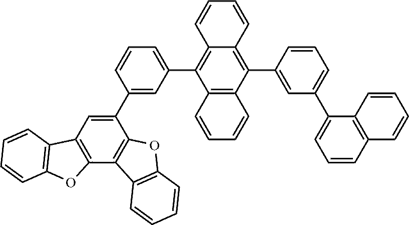 [SMILES: c1cc(-c2cccc3ccccc23)cc(-c2c3ccccc3c(-c3cccc(-c4cc5c6ccccc6oc5c5c4oc4ccccc45)c3)c3ccccc23)c1]

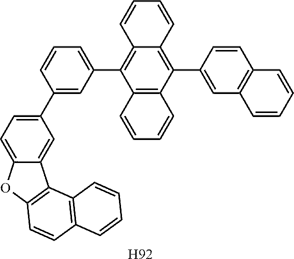

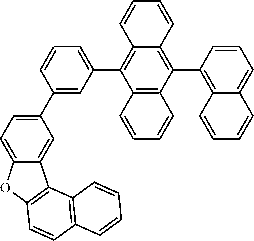 [SMILES: c1cc(-c2ccc3oc4ccc5ccccc5c4c3c2)cc(-c2c3ccccc3c(-c3cccc4ccccc34)c3ccccc23)c1]

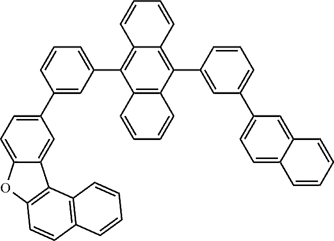 [SMILES: c1cc(-c2ccc3ccccc3c2)cc(-c2c3ccccc3c(-c3cccc(-c4ccc5oc6ccc7ccccc7c6c5c4)c3)c3ccccc23)c1]

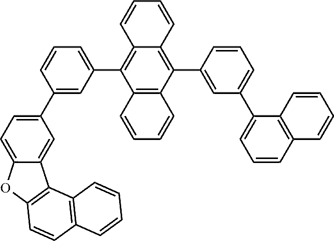 [SMILES: c1cc(-c2ccc3oc4ccc5ccccc5c4c3c2)cc(-c2c3ccccc3c(-c3cccc(-c4cccc5ccccc45)c3)c3ccccc23)c1]

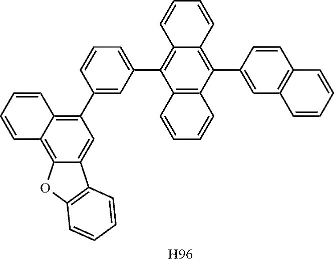 [SMILES: c1cc(-c2c3ccccc3c(-c3ccc4ccccc4c3)c3ccccc23)cc(-c2cc3c4ccccc4oc3c3ccccc23)c1 ; "H96"]

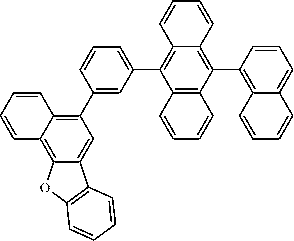 [SMILES: c1cc(-c2c3ccccc3c(-c3cccc4ccccc34)c3ccccc23)cc(-c2cc3c4ccccc4oc3c3ccccc23)c1]

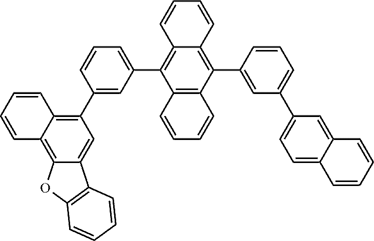 [SMILES: c1cc(-c2ccc3ccccc3c2)cc(-c2c3ccccc3c(-c3cccc(-c4cc5c6ccccc6oc5c5ccccc45)c3)c3ccccc23)c1]

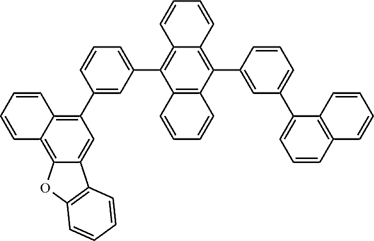 [SMILES: c1cc(-c2cccc3ccccc23)cc(-c2c3ccccc3c(-c3cccc(-c4cc5c6ccccc6oc5c5ccccc45)c3)c3ccccc23)c1]

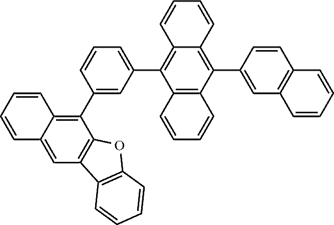 [SMILES: c1cc(-c2c3ccccc3c(-c3ccc4ccccc4c3)c3ccccc23)cc(-c2c3ccccc3cc3c2oc2ccccc23)c1]

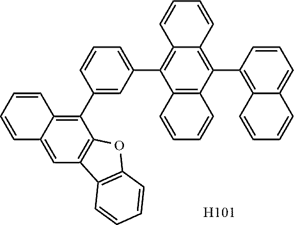 [SMILES: c1cc(-c2c3ccccc3c(-c3cccc4ccccc34)c3ccccc23)cc(-c2c3ccccc3cc3c2oc2ccccc23)c1 ; "H101"]

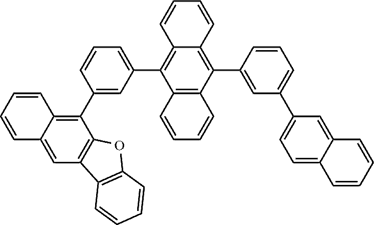 [SMILES: c1cc(-c2ccc3ccccc3c2)cc(-c2c3ccccc3c(-c3cccc(-c4c5ccccc5cc5c4oc4ccccc45)c3)c3ccccc23)c1]

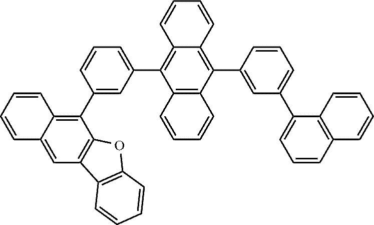 [SMILES: c1cc(-c2cccc3ccccc23)cc(-c2c3ccccc3c(-c3cccc(-c4c5ccccc5cc5c4oc4ccccc45)c3)c3ccccc23)c1]

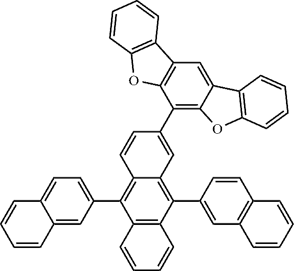 [SMILES: c1ccc2cc(-c3c4ccccc4c(-c4ccc5ccccc5c4)c4cc(-c5c6oc7ccccc7c6cc6c5oc5ccccc56)ccc34)ccc2c1]

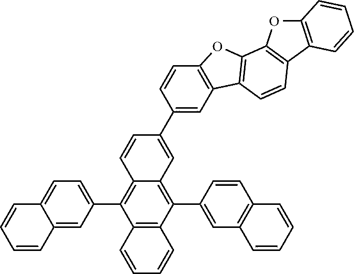 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1]

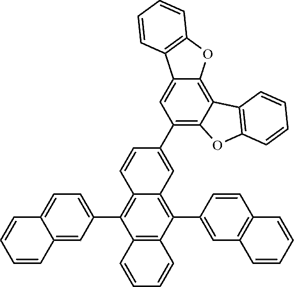 [SMILES: c1ccc2cc(-c3c4ccccc4c(-c4ccc5ccccc5c4)c4cc(-c5cc6c7ccccc7oc6c6c5oc5ccccc56)ccc34)ccc2c1]

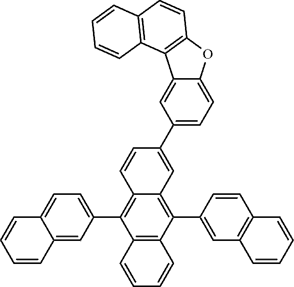 [SMILES: c1ccc2cc(-c3c4ccccc4c(-c4ccc5ccccc5c4)c4cc(-c5ccc6oc7ccc8ccccc8c7c6c5)ccc34)ccc2c1]

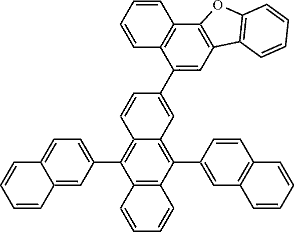 [SMILES: c1ccc2cc(-c3c4ccccc4c(-c4ccc5ccccc5c4)c4cc(-c5cc6c7ccccc7oc6c6ccccc56)ccc34)ccc2c1]

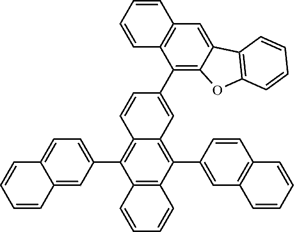 [SMILES: c1ccc2cc(-c3c4ccccc4c(-c4ccc5ccccc5c4)c4cc(-c5c6ccccc6cc6c5oc5ccccc56)ccc34)ccc2c1]

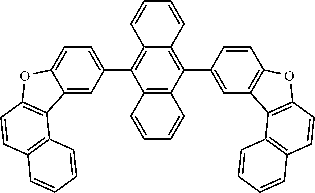 [SMILES: c1ccc2c(c1)ccc1oc3ccc(-c4c5ccccc5c(-c5ccc6oc7ccc8ccccc8c7c6c5)c5ccccc45)cc3c12]

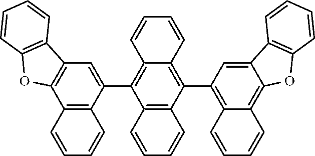 [SMILES: c1ccc2c(c1)oc1c3ccccc3c(-c3c4ccccc4c(-c4cc5c6ccccc6oc5c5ccccc45)c4ccccc34)cc21]

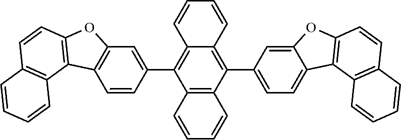 [SMILES: c1ccc2c(c1)ccc1oc3cc(-c4c5ccccc5c(-c5ccc6c(c5)oc5ccc7ccccc7c56)c5ccccc45)ccc3c12]

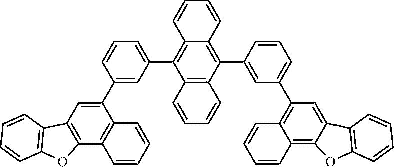 [SMILES: c1cc(-c2c3ccccc3c(-c3cccc(-c4cc5c6ccccc6oc5c5ccccc45)c3)c3ccccc23)cc(-c2cc3c4ccccc4oc3c3ccccc23)c1]

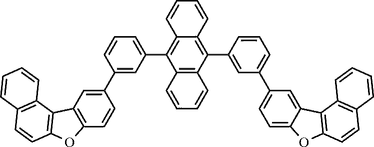 [SMILES: c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1]

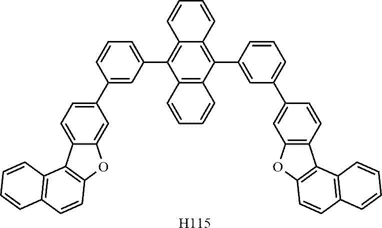 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; "H115"]

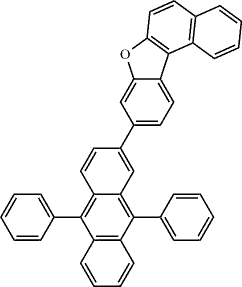 [SMILES: c1ccc(-c2c3ccccc3c(-c3ccccc3)c3cc(-c4ccc5c(c4)oc4ccc6ccccc6c45)ccc23)cc1]

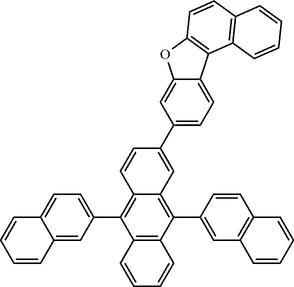 [SMILES: c1ccc2cc(-c3c4ccccc4c(-c4ccc5ccccc5c4)c4cc(-c5ccc6c(c5)oc5ccc7ccccc7c56)ccc34)ccc2c1]

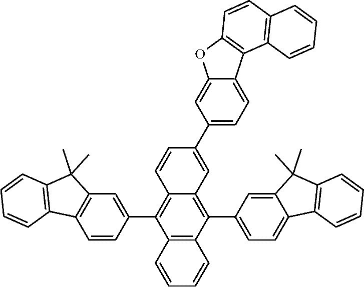 [SMILES: CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

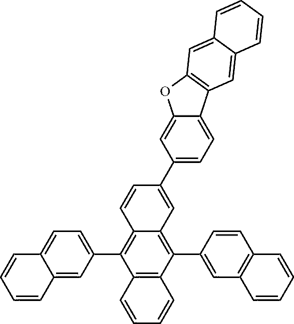 [SMILES: c1ccc2cc(-c3c4ccccc4c(-c4ccc5ccccc5c4)c4cc(-c5ccc6c(c5)oc5cc7ccccc7cc56)ccc34)ccc2c1]

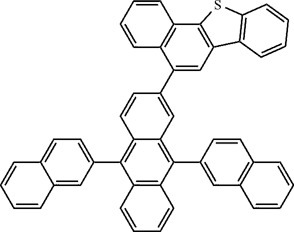 [SMILES: c1ccc2cc(-c3c4ccccc4c(-c4ccc5ccccc5c4)c4cc(-c5cc6c7ccccc7sc6c6ccccc56)ccc34)ccc2c1]

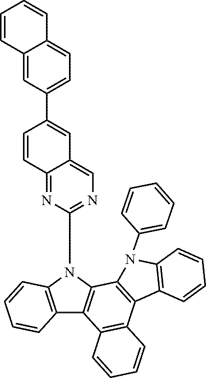 [SMILES: c1ccc(-n2c3ccccc3c3c4ccccc4c4c5ccccc5n(-c5ncc6cc(-c7ccc8ccccc8c7)ccc6n5)c4c32)cc1]

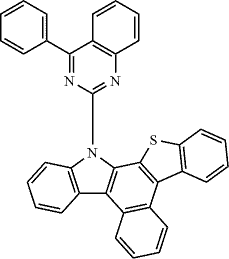 [SMILES: c1ccc(-c2nc(-n3c4ccccc4c4c5ccccc5c5c6ccccc6sc5c43)nc3ccccc23)cc1]

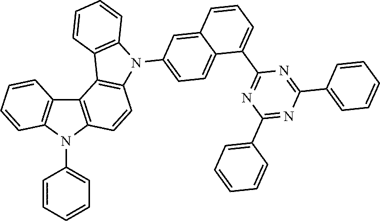 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1]

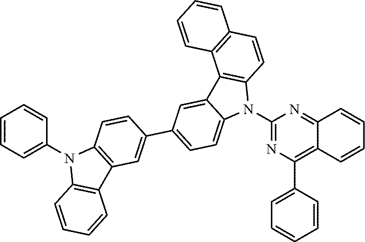 [SMILES: c1ccc(-c2nc(-n3c4ccc(-c5ccc6c(c5)c5ccccc5n6-c5ccccc5)cc4c4c5ccccc5ccc43)nc3ccccc23)cc1]

### Phosphorescent Dopant

The phosphorescent dopant may include at least one transition metal as a central metal atom.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

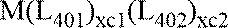

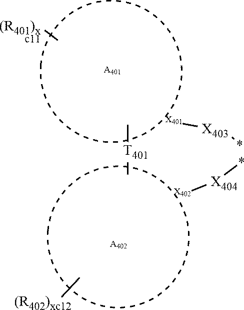

wherein, in Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein, when xc1 is 2 or more, two or more of L₄₀₁ (s) may be identical to or different from each other,
L₄₀₂ may be an organic ligand, and xc2 may be 0, 1, 2, 3, or 4, wherein, when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, —O—, —S—, —C(═O)—, —N(Q₄₁₁)—, —C(Q₄₁₁)(Q₄₁₂)—, —C(Q₄₁₁)═C(Q₄₁₂)—, —C(Q₄₁₁)═, or ═C═,
X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ are each the same as described in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ are each the same as described in connection with Q₁,
xc11 and xc12 may each independently be an integer from 0 to 10, and
* and *’ in Formula 402 each indicate a binding site to M in Formula 401.

For example, in Formula 402, i) X₄₀₁ may be nitrogen, and X₄₀₂ may be carbon, or ii) X₄₀₁ and X₄₀₂ may both be nitrogen.

In one or more embodiments, when xc1 in Formula 401 is 2 or more, two ring A₄₀₁(s) in two or more of L₄₀₁ (s) may optionally be bonded to each other via T₄₀₂, which is a linking group, and two ring A₄₀₂(s) may optionally be bonded to each other via T₄₀₃, which is a linking group (see Compounds PD1 to PD4 and PD7). T₄₀₂ and T₄₀₃ are each the same as described in connection with T₄₀₁.

L₄₀₂ in Formula 401 may be an organic ligand. For example, L₄₀₂ may include a halogen group, a diketone group (for example, an acetylacetonate group), a carboxylic acid group (for example, a picolinate group), —C(═O), an isonitrile group, -CN group, a phosphorus group (for example, a phosphine group, a phosphite group, and/or the like), or any combination thereof.

The phosphorescent dopant may include, for example, one selected from Compounds PD1 to PD39 or any combination thereof:

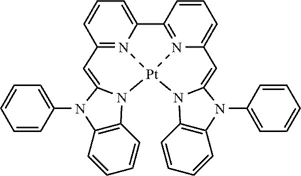

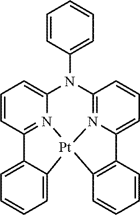

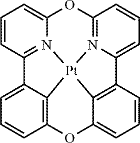

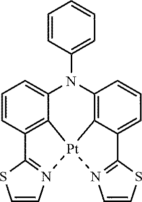

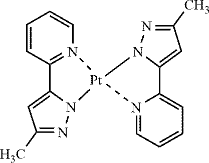

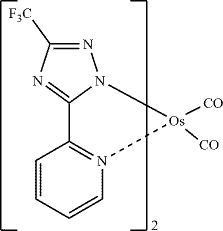

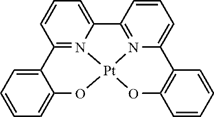

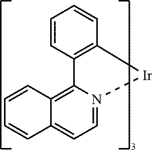

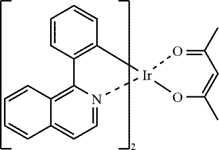

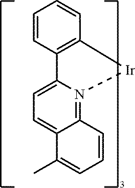

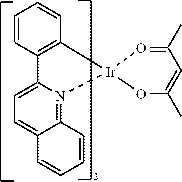

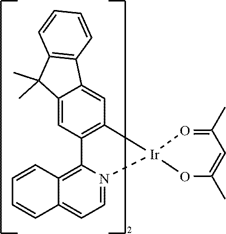

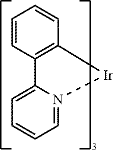

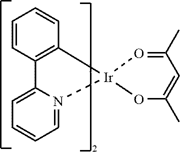

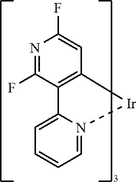

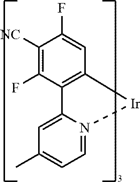

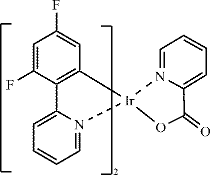

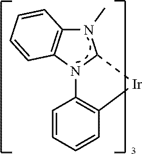

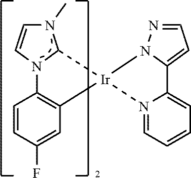

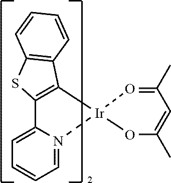

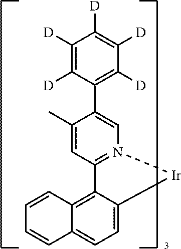

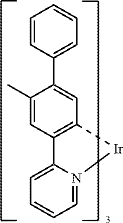

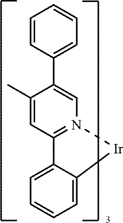

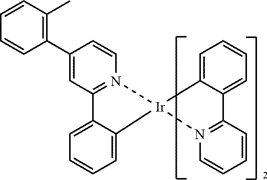

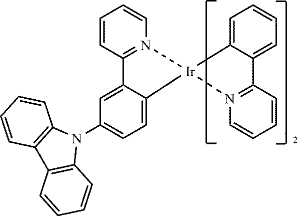

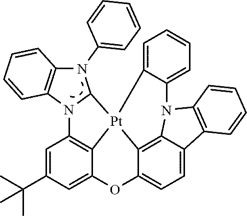

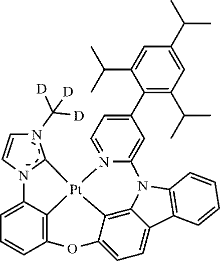

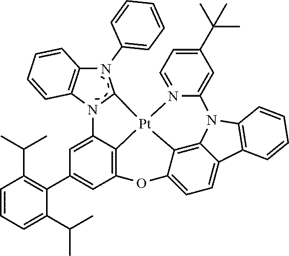

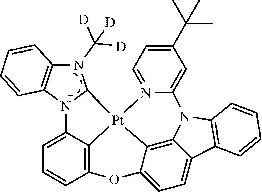

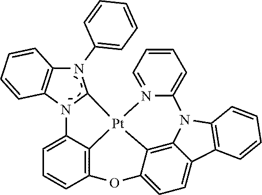

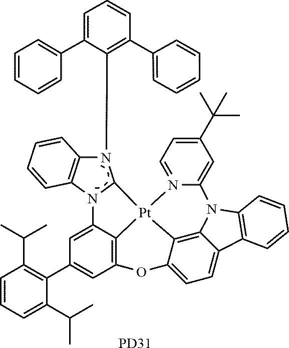

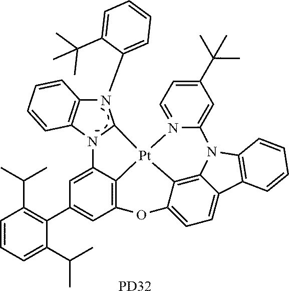

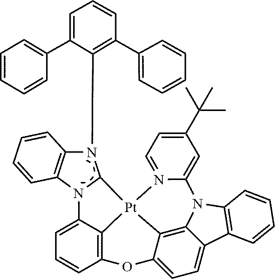

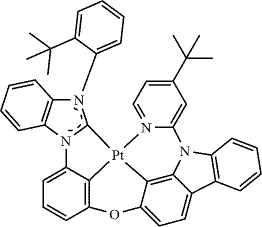

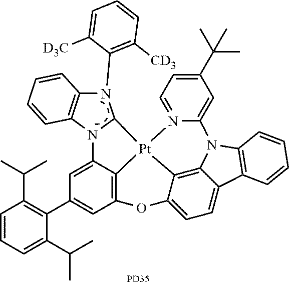

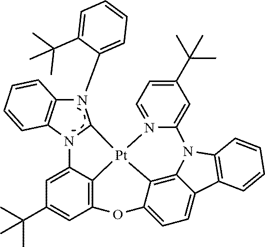

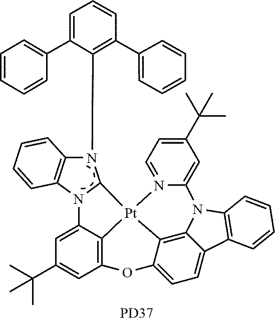

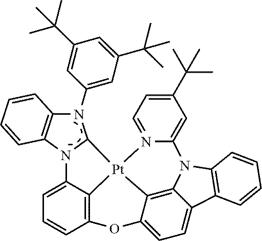

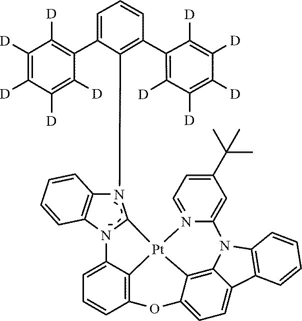

### Fluorescent Dopant

The fluorescent dopant may include an amine group-containing compound, a styryl group-containing compound, or any combination thereof.

For example, the fluorescent dopant may include a compound represented by Formula 501:

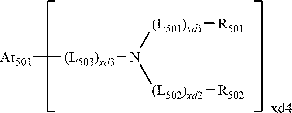

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, Ar₅₀₁ in Formula 501 may be a condensed cyclic group (for example, an anthracene group, a chrysene group, a pyrene group, or the like) in which three or more monocyclic groups are condensed together.

In one or more embodiments, xd4 in Formula 501 may be 2.

For example, the fluorescent dopant may include one selected from Compounds FD1 to FD36, DPVBi, DPAVBi, or any combination thereof:

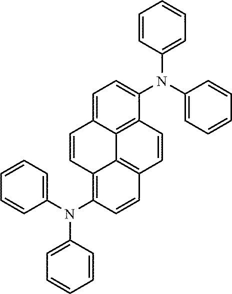 [SMILES: c1ccc(N(c2ccccc2)c2ccc3ccc4c(N(c5ccccc5)c5ccccc5)ccc5ccc2c3c54)cc1]

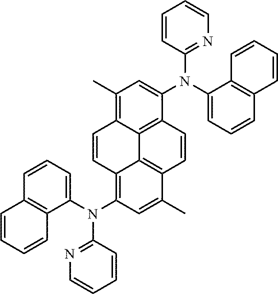 [SMILES: Cc1cc(N(c2ccccn2)c2cccc3ccccc23)c2ccc3c(C)cc(N(c4ccccn4)c4cccc5ccccc45)c4ccc1c2c34]

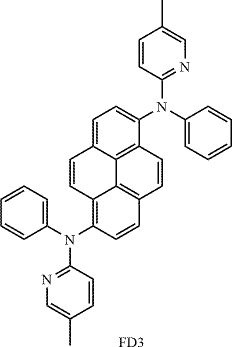

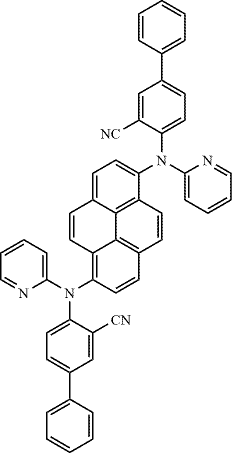

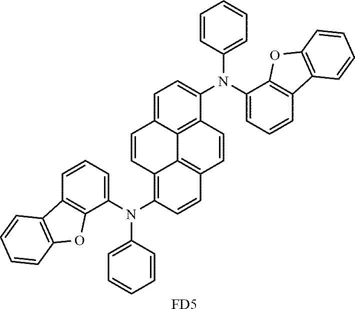

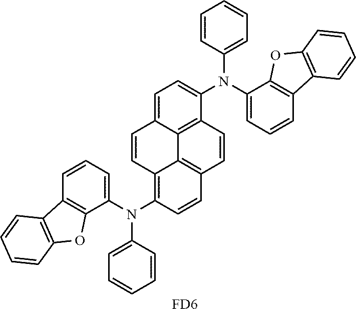

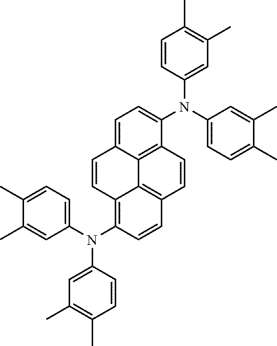

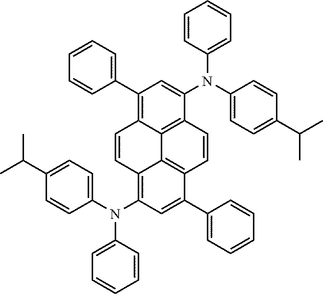 [SMILES: CC(C)c1ccc(N(c2ccccc2)c2cc(-c3ccccc3)c3ccc4c(N(c5ccccc5)c5ccc(C(C)C)cc5)cc(-c5ccccc5)c5ccc2c3c54)cc1]

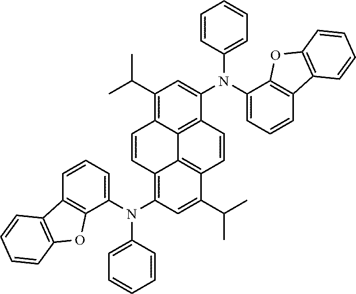 [SMILES: CC(C)c1cc(N(c2ccccc2)c2cccc3c2oc2ccccc23)c2ccc3c(C(C)C)cc(N(c4ccccc4)c4cccc5c4oc4ccccc45)c4ccc1c2c34]

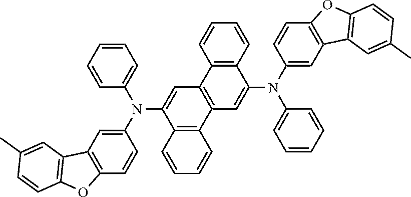

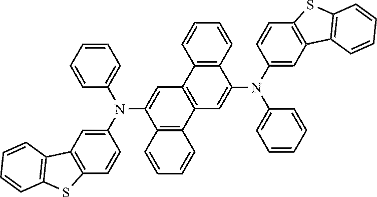 [SMILES: c1ccc(N(c2ccc3sc4ccccc4c3c2)c2cc3c4ccccc4c(N(c4ccccc4)c4ccc5sc6ccccc6c5c4)cc3c3ccccc23)cc1]

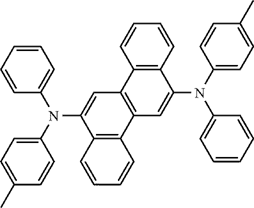 [SMILES: Cc1ccc(N(c2ccccc2)c2cc3c4ccccc4c(N(c4ccccc4)c4ccc(C)cc4)cc3c3ccccc23)cc1]

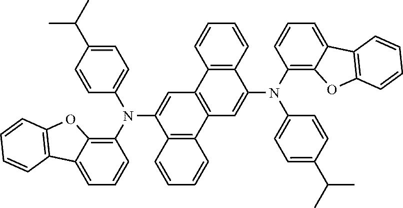 [SMILES: CC(C)c1ccc(N(c2cc3c4ccccc4c(N(c4ccc(C(C)C)cc4)c4cccc5c4oc4ccccc45)cc3c3ccccc23)c2cccc3c2oc2ccccc23)cc1]

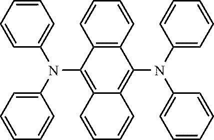 [SMILES: c1ccc(N(c2ccccc2)c2c3ccccc3c(N(c3ccccc3)c3ccccc3)c3ccccc23)cc1]

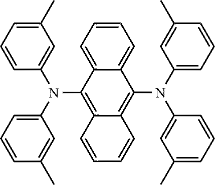 [SMILES: Cc1cccc(N(c2cccc(C)c2)c2c3ccccc3c(N(c3cccc(C)c3)c3cccc(C)c3)c3ccccc23)c1]

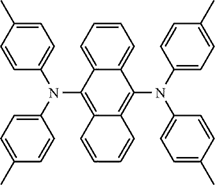 [SMILES: Cc1ccc(N(c2ccc(C)cc2)c2c3ccccc3c(N(c3ccc(C)cc3)c3ccc(C)cc3)c3ccccc23)cc1]

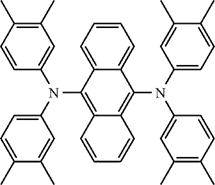 [SMILES: Cc1ccc(N(c2ccc(C)c(C)c2)c2c3ccccc3c(N(c3ccc(C)c(C)c3)c3ccc(C)c(C)c3)c3ccccc23)cc1C]

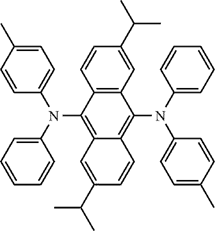 [SMILES: Cc1ccc(N(c2ccccc2)c2c3ccc(C(C)C)cc3c(N(c3ccccc3)c3ccc(C)cc3)c3ccc(C(C)C)cc23)cc1]

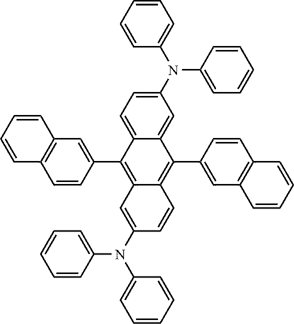 [SMILES: c1ccc(N(c2ccccc2)c2ccc3c(-c4ccc5ccccc5c4)c4cc(N(c5ccccc5)c5ccccc5)ccc4c(-c4ccc5ccccc5c4)c3c2)cc1]

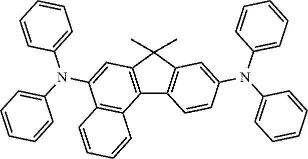 [SMILES: CC1(C)c2cc(N(c3ccccc3)c3ccccc3)ccc2-c2c1cc(N(c1ccccc1)c1ccccc1)c1ccccc21]

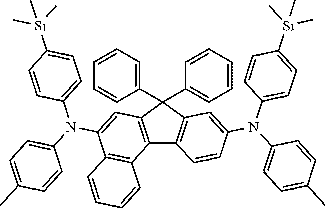

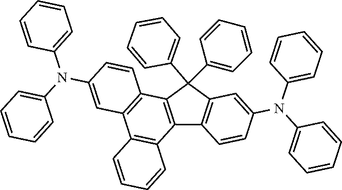 [SMILES: c1ccc(N(c2ccccc2)c2ccc3c(c2)C(c2ccccc2)(c2ccccc2)c2c-3c3ccccc3c3cc(N(c4ccccc4)c4ccccc4)ccc23)cc1]

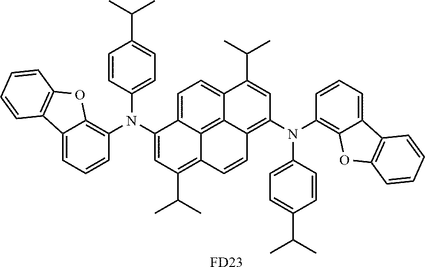

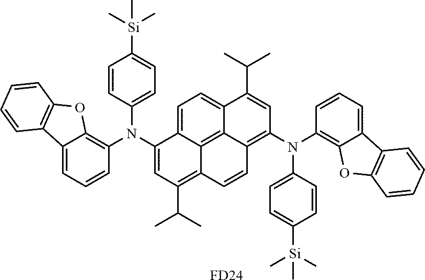

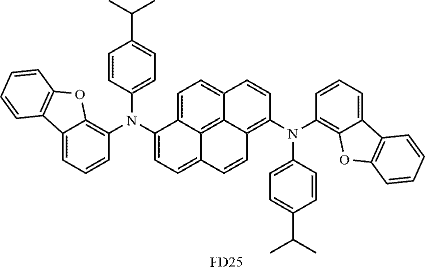 [SMILES: CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1 ; "FD25"]

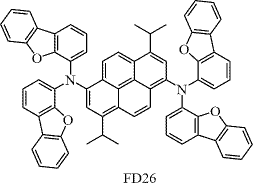

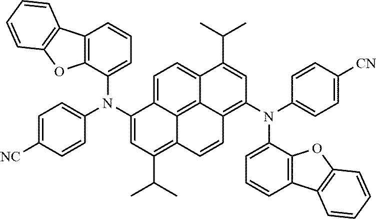

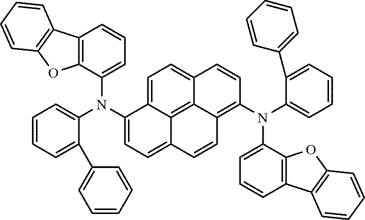 [SMILES: c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1]

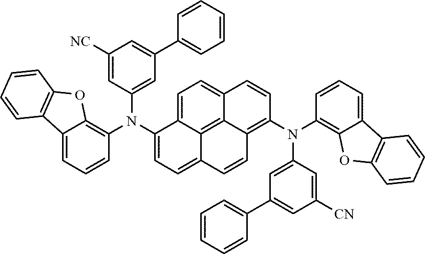

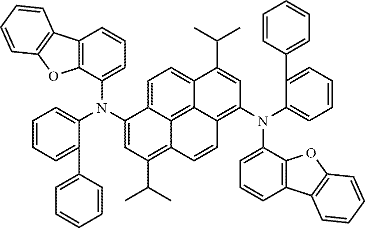 [SMILES: CC(C)c1cc(N(c2ccccc2-c2ccccc2)c2cccc3c2oc2ccccc23)c2ccc3c(C(C)C)cc(N(c4ccccc4-c4ccccc4)c4cccc5c4oc4ccccc45)c4ccc1c2c34]

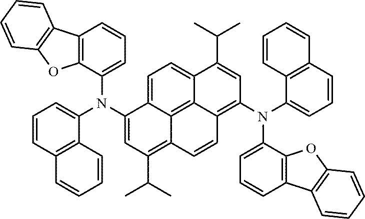 [SMILES: CC(C)c1cc(N(c2cccc3ccccc23)c2cccc3c2oc2ccccc23)c2ccc3c(C(C)C)cc(N(c4cccc5ccccc45)c4cccc5c4oc4ccccc45)c4ccc1c2c34]

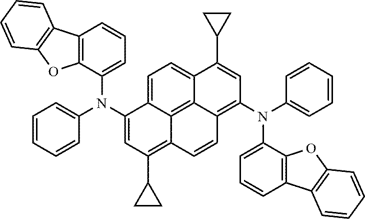 [SMILES: c1ccc(N(c2cc(C3CC3)c3ccc4c(N(c5ccccc5)c5cccc6c5oc5ccccc56)cc(C5CC5)c5ccc2c3c54)c2cccc3c2oc2ccccc23)cc1]

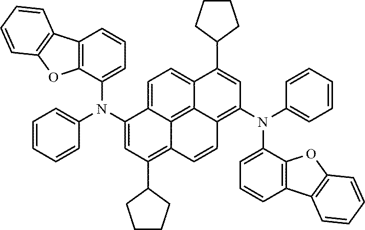 [SMILES: c1ccc(N(c2cc(C3CCCC3)c3ccc4c(N(c5ccccc5)c5cccc6c5oc5ccccc56)cc(C5CCCC5)c5ccc2c3c54)c2cccc3c2oc2ccccc23)cc1]

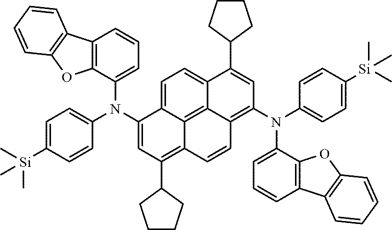

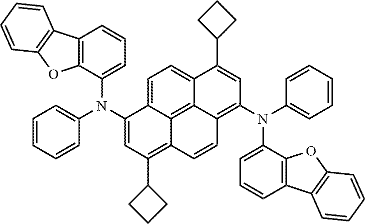 [SMILES: c1ccc(N(c2cc(C3CCC3)c3ccc4c(N(c5ccccc5)c5cccc6c5oc5ccccc56)cc(C5CCC5)c5ccc2c3c54)c2cccc3c2oc2ccccc23)cc1]

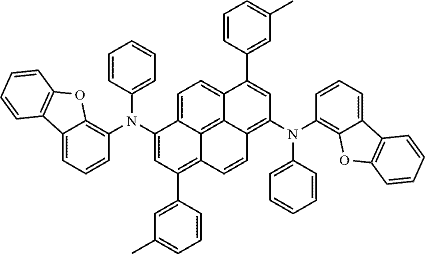 [SMILES: Cc1cccc(-c2cc(N(c3ccccc3)c3cccc4c3oc3ccccc34)c3ccc4c(-c5cccc(C)c5)cc(N(c5ccccc5)c5cccc6c5oc5ccccc56)c5ccc2c3c45)c1]

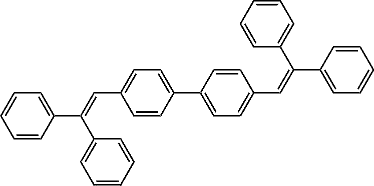 [SMILES: C(=C(c1ccccc1)c1ccccc1)c1ccc(-c2ccc(C=C(c3ccccc3)c3ccccc3)cc2)cc1]

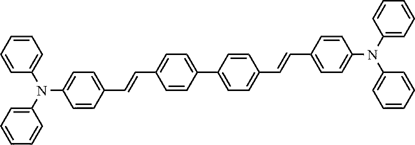 [SMILES: C(=C\c1ccc(N(c2ccccc2)c2ccccc2)cc1)/c1ccc(-c2ccc(/C=C/c3ccc(N(c4ccccc4)c4ccccc4)cc3)cc2)cc1]

### Delayed Fluorescence Material

The emission layer may include a delayed fluorescence material.

In the present specification, the delayed fluorescence material may be selected from compounds capable of emitting delayed fluorescent light based on a delayed fluorescence emission mechanism.

The delayed fluorescence material included in the emission layer may act as a host or a dopant depending on the type (or kind) of other materials included in the emission layer.

In an embodiment, a difference between a triplet energy level (eV) of the delayed fluorescence material and a singlet energy level (eV) of the delayed fluorescence material may be equal to or greater than 0 eV and equal to or less than 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively occur, and thus, the luminescence efficiency of the light-emitting device 10 may be improved.

For example, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, or a π electron-deficient nitrogen-containing C₁-C₆₀ cyclic group), and ii) a material including a C₈-C₆₀ polycyclic group in which two or more cyclic groups are condensed together while sharing boron (B).

Examples of the delayed fluorescence material may include at least one selected from Compounds DF1 to DF9:

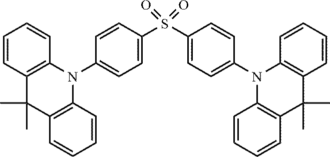

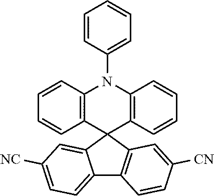

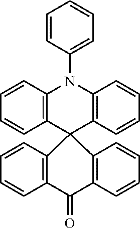 [SMILES: O=C1c2ccccc2C2(c3ccccc31)c1ccccc1N(c1ccccc1)c1ccccc12]

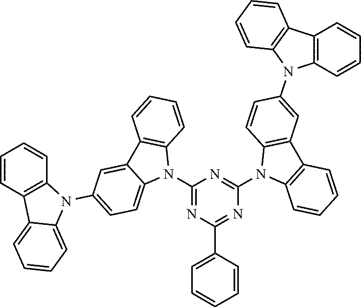 [SMILES: c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1]

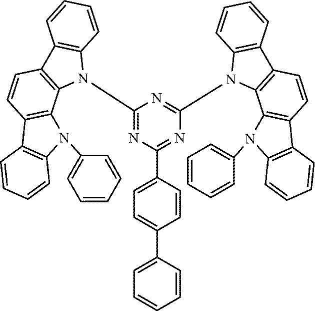 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1]

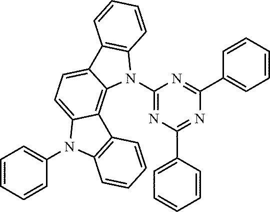 [SMILES: c1ccc(-c2nc(-c3ccccc3)nc(-n3c4ccccc4c4ccc5c(c6ccccc6n5-c5ccccc5)c43)n2)cc1]

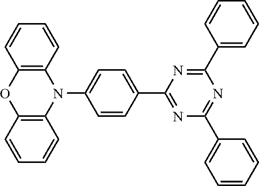 [SMILES: c1ccc(-c2nc(-c3ccccc3)nc(-c3ccc(N4c5ccccc5Oc5ccccc54)cc3)n2)cc1]

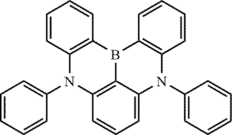 [SMILES: c1ccc(N2c3ccccc3B3c4ccccc4N(c4ccccc4)c4cccc2c43)cc1]

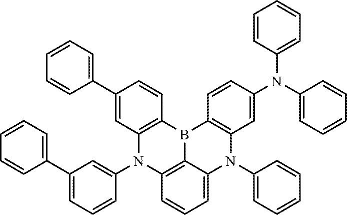 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1]

### Quantum Dot

The emission layer may include a quantum dot.

The term “quantum dot,” as used herein, refers to a crystal of a semiconductor compound, and may include any suitable material capable of emitting light of various suitable emission wavelengths according to the size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, and/or any suitable process similar thereto.

The wet chemical process is a method including mixing together a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled through a process which costs lower, and is easier than vapor deposition methods, such as metal organic chemical vapor deposition (MOCVD) or molecular beam epitaxy (MBE).

The quantum dot may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Examples of the Group II-VI semiconductor compound may include: a binary compound, such as CdS, CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or any combination thereof.

Examples of the Group III-V semiconductor compound may include: a binary compound, such as GaN, GaP, GaAs, GaSb, AIN, AIP, AlAs, AlSb, InN, InP, InAs, and/or InSb; a ternary compound, such as GaNP, GaNAs, GaNSb, GaPAs, GaPSb, AINP, AINAs, AINSb, AlPAs, AlPSb, InGaP, InNP, InAlP, InNAs, InNSb, InPAs, and/or InPSb; a quaternary compound, such as GaAINP, GaAINAs, GaAINSb, GaAlPAs, GaAlPSb, GaInNP, GaInNAs, GaInNSb, GaInPAs, GaInPSb, InAINP, InAINAs, InAINSb, InAIPAs, and/or InAIPSb; or any combination thereof. In an embodiment, the Group III-V semiconductor compound may further include a Group II element. Examples of the Group III-V semiconductor compound further including the Group II element may include InZnP, InGaZnP, InAIZnP, and/or the like.

Examples of the Group III-VI semiconductor compound may include: a binary compound, such as GaS, GaSe, Ga₂Se₃, GaTe, InS, InSe, In₂S₃, In₂Se₃, and/or InTe; a ternary compound, such as InGaS₃ and/or InGaSe₃; or any combination thereof.

Examples of the Group I-III-VI semiconductor compound may include: a ternary compound, such as AgInS, AgInS₂, CuInS, CuInS₂, CuGaO₂, AgGaO₂, and/or AgAlO₂; or any combination thereof.

Examples of the Group IV-VI semiconductor compound may include: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or any combination thereof.

The Group IV element or compound may include: a single element compound, such as Si and/or Ge; a binary compound, such as SiC and/or SiGe; or any combination thereof.

Each element included in a multi-element compound such as the binary compound, the ternary compound, and the quaternary compound may be present at a uniform concentration or non-uniform concentration in a particle.

In an embodiment, the quantum dot may have a single structure in which the concentration of each element in the quantum dot is uniform (e.g., substantially uniform), or a core-shell dual structure. For example, materials included in the core may be different from materials included in the shell.

The shell of the quantum dot may act as a protective layer that prevents or reduces chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases along a direction toward the center of the core.

Examples of the shell of the quantum dot may include oxide of a metal, a metalloid, and/or a non-metal, a semiconductor compound, or a combination thereof. Examples of the oxide of the metal, the metalloid, and the non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO; a ternary compound, such as MgAl₂O₄, CoFe₂O₄, NiFe₂O₄, and/or CoMn₂O₄; or any combination thereof. Examples of the semiconductor compound may include, as described herein, a Group II-VI semiconductor compound; a Group III-V semiconductor compound; a Group III-VI semiconductor compound; a Group I-III-VI semiconductor compound; a Group IV-VI semiconductor compound; or any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AIP, AlSb, or any combination thereof.

A full width at half maximum (FWHM) of an emission wavelength spectrum of the quantum dot may be about 45 nm or less, for example, about 40 nm or less, for example, about 30 nm or less, and within these ranges, color purity or color reproducibility may be improved. In addition, because light emitted through the quantum dots is emitted in all directions (e.g., substantially all directions), an optical viewing angle may be improved.

In addition, the quantum dot may be in the form of a spherical particle, a pyramidal particle, a multi-arm particle, a cubic nanoparticle, a nanotube particle, a nanowire particle, a nanofiber particle, and/or a nanoplate particle.

Because the energy band gap may be adjusted by controlling the size of the quantum dot, light having various suitable wavelength bands may be obtained from the quantum dot emission layer. Accordingly, by using quantum dots of different sizes, a light-emitting device that emits light of various suitable wavelengths may be implemented. In an embodiment, the size of the quantum dot may be selected to emit red, green and/or blue light. In addition, the size of the quantum dot may be configured to emit white light by combination of light of various suitable colors.

### Electron Transport Region in Interlayer 130

The electron transport region may have i) a single-layered structure consisting of a single layer consisting of a single material, ii) a single-layered structure consisting of a single layer consisting of a plurality of different materials, or iii) a multi-layered structure including a plurality of layers including different materials.

The electron transport region may include a hole blocking layer, an electron transport layer, an electron injection layer, or any combination thereof.

For example, the electron transport region may have an electron transport layer/electron injection layer structure or a hole blocking layer/electron transport layer/electron injection layer structure, wherein, in each structure, constituting layers are sequentially stacked from the emission layer.

The electron transport region (for example, the hole blocking layer or the electron transport layer in the electron transport region) may include a metal-free compound including at least one π electron-deficient nitrogen-containing C₁-C₆₀ cyclic group.

For example, the electron transport region may include a compound represented by Formula 601:

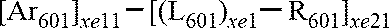

wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(O₆₀₁), or —P(═O)(Q₆₀₁)(Q₆₀₂),
Q₆₀₁ to Q₆₀₃ are each the same as described in connection with Q₁,
xe21 may be 1, 2, 3, 4, or 5, and
at least one selected from Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁ (s) may be bonded to each other via a single bond.

In one or more embodiments, Ar₆₀₁ in Formula 601 may be a substituted or unsubstituted anthracene group.

In one or more embodiments, the electron transport region may include a compound represented by Formula 601-1:

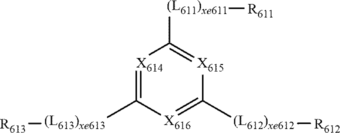

wherein, in Formula 601-1,
X₆₁₄ may be N or C(R₆₁₄), X₆₁₅ may be N or C(R₆₁₅), X₆₁₆ may be N or C(R₆₁₆), and at least one selected from X₆₁₄ to X₆₁₆ may be N,
L₆₁₁ to L₆₁₃ are each the same as described in connection with L₆₀₁,
xe611 to xe613 are each the same as described in connection with xe1,
R₆₁₁ to R₆₁₃ are each the same as described in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one selected from Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

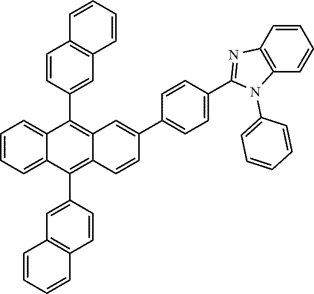 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

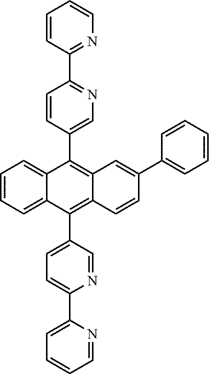 [SMILES: c1ccc(-c2ccc3c(-c4ccc(-c5ccccn5)nc4)c4ccccc4c(-c4ccc(-c5ccccn5)nc4)c3c2)cc1]

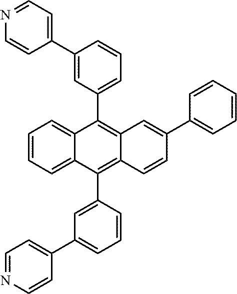 [SMILES: c1ccc(-c2ccc3c(-c4cccc(-c5ccncc5)c4)c4ccccc4c(-c4cccc(-c5ccncc5)c4)c3c2)cc1]

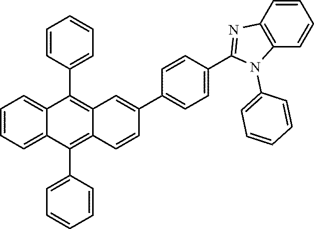 [SMILES: c1ccc(-c2c3ccccc3c(-c3ccccc3)c3cc(-c4ccc(-c5nc6ccccc6n5-c5ccccc5)cc4)ccc23)cc1]

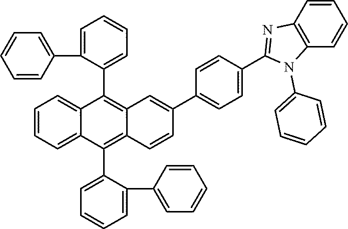 [SMILES: c1ccc(-c2ccccc2-c2c3ccccc3c(-c3ccccc3-c3ccccc3)c3cc(-c4ccc(-c5nc6ccccc6n5-c5ccccc5)cc4)ccc23)cc1]

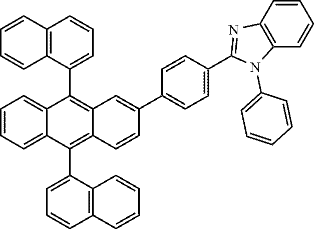 [SMILES: c1ccc(-n2c(-c3ccc(-c4ccc5c(-c6cccc7ccccc67)c6ccccc6c(-c6cccc7ccccc67)c5c4)cc3)nc3ccccc32)cc1]

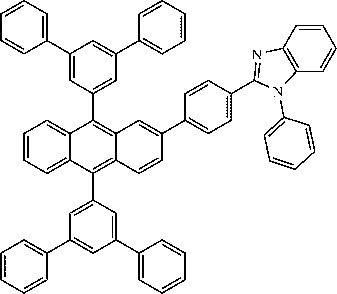 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

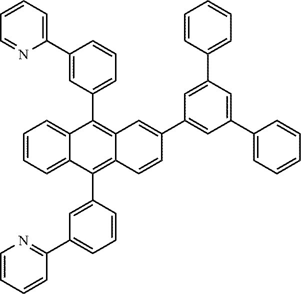 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1]

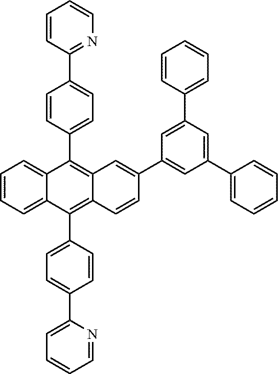 [SMILES: c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

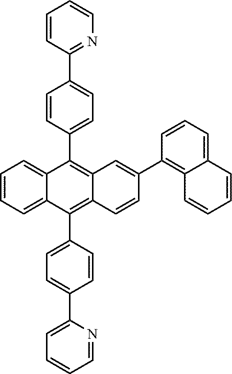 [SMILES: c1ccc(-c2ccc(-c3c4ccccc4c(-c4ccc(-c5ccccn5)cc4)c4cc(-c5cccc6ccccc56)ccc34)cc2)nc1]

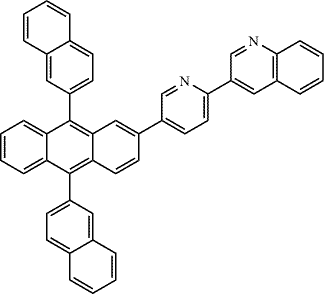 [SMILES: c1ccc2cc(-c3c4ccccc4c(-c4ccc5ccccc5c4)c4cc(-c5ccc(-c6cnc7ccccc7c6)nc5)ccc34)ccc2c1]

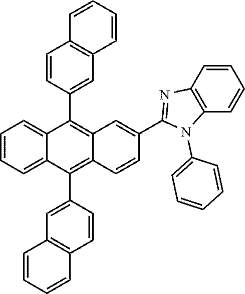 [SMILES: c1ccc(-n2c(-c3ccc4c(-c5ccc6ccccc6c5)c5ccccc5c(-c5ccc6ccccc6c5)c4c3)nc3ccccc32)cc1]

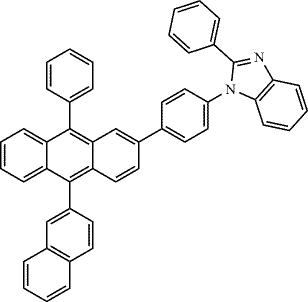 [SMILES: c1ccc(-c2c3ccccc3c(-c3ccc4ccccc4c3)c3ccc(-c4ccc(-n5c(-c6ccccc6)nc6ccccc65)cc4)cc23)cc1]

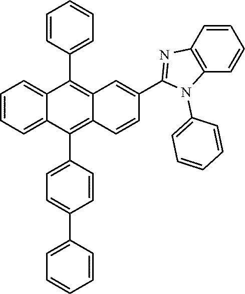 [SMILES: c1ccc(-c2ccc(-c3c4ccccc4c(-c4ccccc4)c4cc(-c5nc6ccccc6n5-c5ccccc5)ccc34)cc2)cc1]

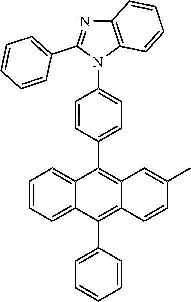 [SMILES: Cc1ccc2c(-c3ccccc3)c3ccccc3c(-c3ccc(-n4c(-c5ccccc5)nc5ccccc54)cc3)c2c1]

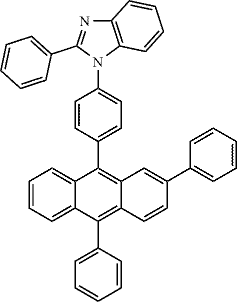 [SMILES: c1ccc(-c2ccc3c(-c4ccccc4)c4ccccc4c(-c4ccc(-n5c(-c6ccccc6)nc6ccccc65)cc4)c3c2)cc1]

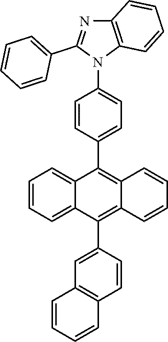 [SMILES: c1ccc(-c2nc3ccccc3n2-c2ccc(-c3c4ccccc4c(-c4ccc5ccccc5c4)c4ccccc34)cc2)cc1]

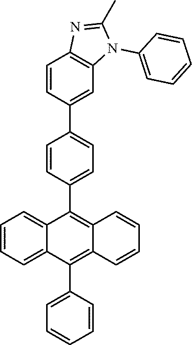 [SMILES: Cc1nc2ccc(-c3ccc(-c4c5ccccc5c(-c5ccccc5)c5ccccc45)cc3)cc2n1-c1ccccc1]

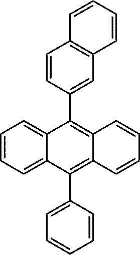 [SMILES: c1ccc(-c2c3ccccc3c(-c3ccc4ccccc4c3)c3ccccc23)cc1]

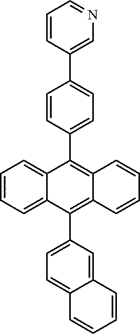 [SMILES: c1cncc(-c2ccc(-c3c4ccccc4c(-c4ccc5ccccc5c4)c4ccccc34)cc2)c1]

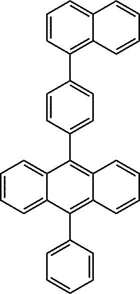 [SMILES: c1ccc(-c2c3ccccc3c(-c3ccc(-c4cccc5ccccc45)cc3)c3ccccc23)cc1]

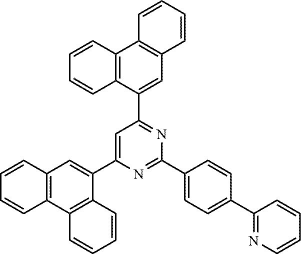 [SMILES: c1ccc(-c2ccc(-c3nc(-c4cc5ccccc5c5ccccc45)cc(-c4cc5ccccc5c5ccccc45)n3)cc2)nc1]

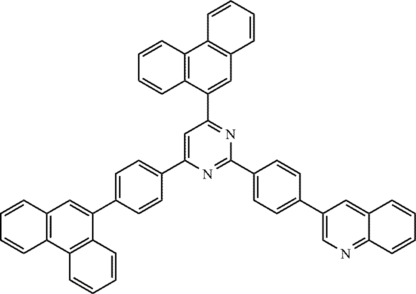 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1]

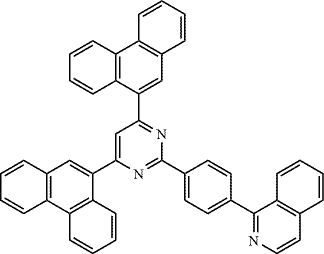 [SMILES: c1ccc2c(-c3ccc(-c4nc(-c5cc6ccccc6c6ccccc56)cc(-c5cc6ccccc6c6ccccc56)n4)cc3)nccc2c1]

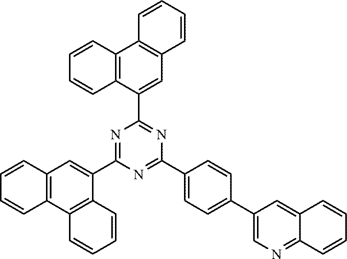 [SMILES: c1ccc2ncc(-c3ccc(-c4nc(-c5cc6ccccc6c6ccccc56)nc(-c5cc6ccccc6c6ccccc56)n4)cc3)cc2c1]

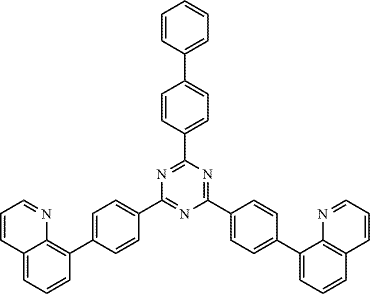 [SMILES: c1ccc(-c2ccc(-c3nc(-c4ccc(-c5cccc6cccnc56)cc4)nc(-c4ccc(-c5cccc6cccnc56)cc4)n3)cc2)cc1]

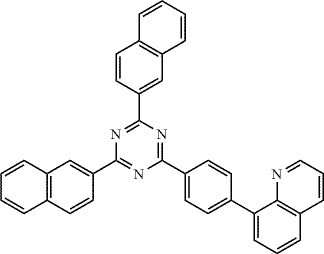 [SMILES: c1ccc2cc(-c3nc(-c4ccc(-c5cccc6cccnc56)cc4)nc(-c4ccc5ccccc5c4)n3)ccc2c1]

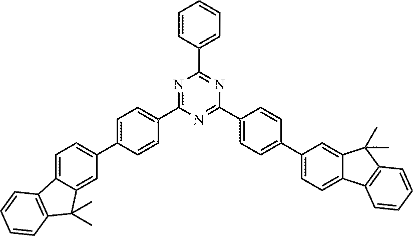 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

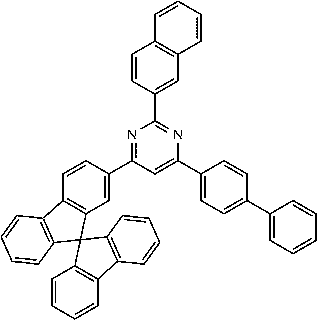 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

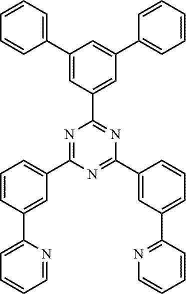 [SMILES: c1ccc(-c2cc(-c3ccccc3)cc(-c3nc(-c4cccc(-c5ccccn5)c4)nc(-c4cccc(-c5ccccn5)c4)n3)c2)cc1]

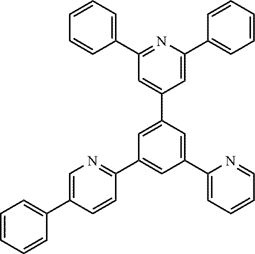 [SMILES: c1ccc(-c2ccc(-c3cc(-c4cc(-c5ccccc5)nc(-c5ccccc5)c4)cc(-c4ccccn4)c3)nc2)cc1]

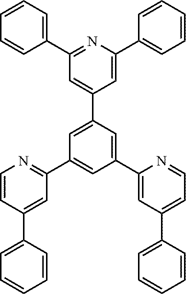 [SMILES: c1ccc(-c2ccnc(-c3cc(-c4cc(-c5ccccc5)nc(-c5ccccc5)c4)cc(-c4cc(-c5ccccc5)ccn4)c3)c2)cc1]

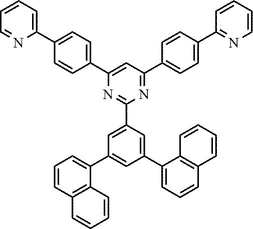 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

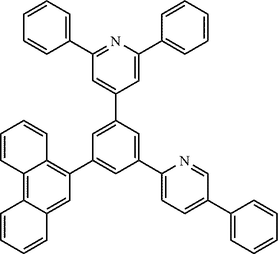 [SMILES: c1ccc(-c2ccc(-c3cc(-c4cc(-c5ccccc5)nc(-c5ccccc5)c4)cc(-c4cc5ccccc5c5ccccc45)c3)nc2)cc1]

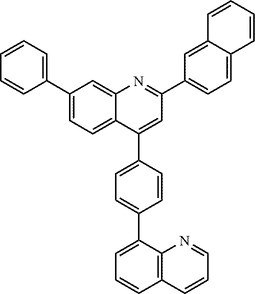 [SMILES: c1ccc(-c2ccc3c(-c4ccc(-c5cccc6cccnc56)cc4)cc(-c4ccc5ccccc5c4)nc3c2)cc1]

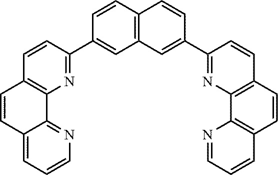 [SMILES: c1cnc2c(c1)ccc1ccc(-c3ccc4ccc(-c5ccc6ccc7cccnc7c6n5)cc4c3)nc12]

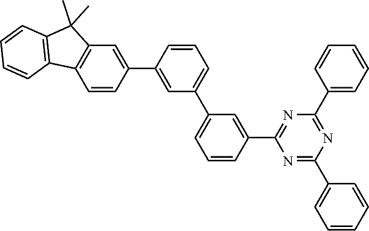 [SMILES: CC1(C)c2ccccc2-c2ccc(-c3cccc(-c4cccc(-c5nc(-c6ccccc6)nc(-c6ccccc6)n5)c4)c3)cc21]

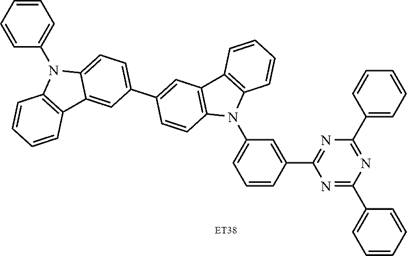

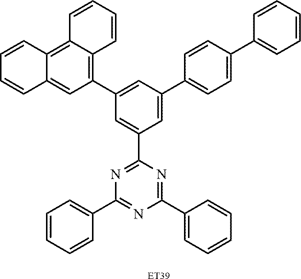

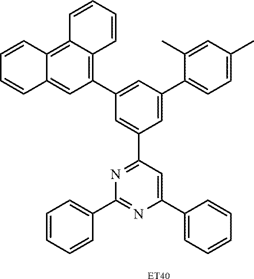

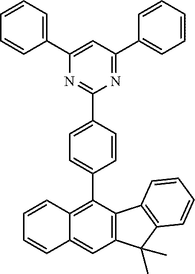 [SMILES: CC1(C)c2ccccc2-c2c1cc1ccccc1c2-c1ccc(-c2nc(-c3ccccc3)cc(-c3ccccc3)n2)cc1]

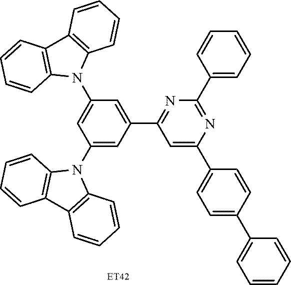

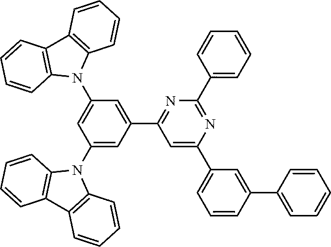 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

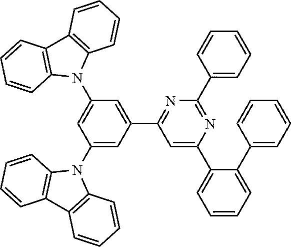 [SMILES: c1ccc(-c2nc(-c3cc(-n4c5ccccc5c5ccccc54)cc(-n4c5ccccc5c5ccccc54)c3)cc(-c3ccccc3-c3ccccc3)n2)cc1]

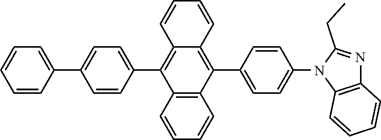 [SMILES: CCc1nc2ccccc2n1-c1ccc(-c2c3ccccc3c(-c3ccc(-c4ccccc4)cc3)c3ccccc23)cc1]

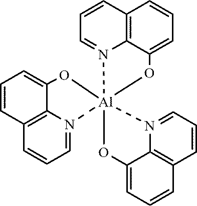

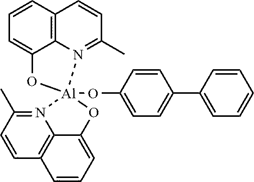

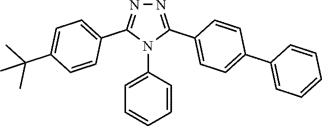 [SMILES: CC(C)(C)c1ccc(-c2nnc(-c3ccc(-c4ccccc4)cc3)n2-c2ccccc2)cc1]

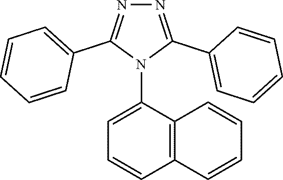 [SMILES: c1ccc(-c2nnc(-c3ccccc3)n2-c2cccc3ccccc23)cc1]

A thickness of the electron transport region may be in range of about 100 Å to about 5,000 Å, for example, about 160 Å to about 4,000 Å. When the electron transport region includes the hole blocking layer, the electron transport layer, or any combination thereof, a thickness of the hole blocking layer or electron transport layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å.When the thicknesses of the hole blocking layer and/or the electron transport layer are within the ranges described above, suitable or satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, the metal-containing material may include a Li complex. The Li complex may include, for example, Compound ET-D1 (LiQ) and/or ET-D2:

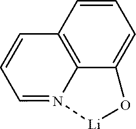

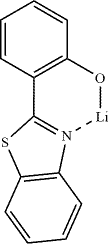

The electron transport region may include an electron injection layer that facilitates the injection of electrons from the second electrode 150. The electron injection layer may be in direct contact (e.g., physical contact) with the second electrode 150.

The electron injection layer may have i) a single-layered structure consisting of a single layer consisting of a single material, ii) a single-layered structure consisting of a single layer consisting of a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (for example, fluorides, chlorides, bromides, and/or iodides), and/or tellurides of the alkali metal, the alkaline earth metal, and the rare earth metal, or any combination thereof.

The alkali metal-containing compound may include: alkali metal oxides, such as Li₂O, Cs₂O, and/or K₂O; alkali metal halides, such as LiF, NaF, CsF, KF, Lil, Nal, Csl, and/or KI; or any combination thereof. The alkaline earth metal-containing compound may include an alkaline earth metal compound, such as BaO, SrO, CaO, BaₓSr₁₋ₓO (wherein x is a real number satisfying the condition of 0<x<1), BaₓCa₁₋ₓO (wherein x is a real number satisfying the condition of 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one selected from ions of the alkali metal, the alkaline earth metal, and the rare earth metal and ii), as a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In an embodiment, the electron injection layer may include (e.g., consist of) i) an alkali metal-containing compound (for example, alkali metal halide), ii) a) an alkali metal-containing compound (for example, alkali metal halide); and b) an alkali metal, an alkaline earth metal, a rare earth metal, or any combination thereof. For example, the electron injection layer may be a KI:Yb co-deposited layer, an Rbl:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å, for example, about 3 Å to about 90 Å. When the thickness of the electron injection layer is within the ranges described above, suitable or satisfactory electron injection characteristics may be obtained without a substantial increase in driving voltage.

### Second Electrode 150

The second electrode 150 may be on the interlayer 130 as described above. The second electrode 150 may be a cathode, which is an electron injection electrode, and a material for forming the second electrode 150 may include a metal, an alloy, an electrically conductive compound, or any combination thereof, each having a low-work function.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be outside the first electrode 110, and/or a second capping layer may be outside the second electrode 150. In more detail, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in the emission layer of the interlayer 130 of the light-emitting device 10 may be extracted toward the outside through the first electrode 110, which is a semi-transmissive electrode or a transmissive electrode, and the first capping layer. Light generated in the emission layer of the interlayer 130 of the light-emitting device 10 may be extracted toward the outside through the second electrode 150, which is a semi-transmissive electrode or a transmissive electrode, and the second capping layer.

The first capping layer and the second capping layer may increase external luminescence efficiency according to the principle of constructive interference. Accordingly, the light extraction efficiency of the light-emitting device 10 may be increased, so that the luminescence efficiency of the light-emitting device 10 may be improved.

Each of the first capping layer and the second capping layer may include a material having a refractive index of 1.6 or more (at a wavelength of 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent containing O, N, S, Se, Si, F, Cl, Br, I, or any combination thereof. In an embodiment, at least one selected from the first capping layer and the second capping layer may each independently include an amine group-containing compound.

For example, at least one selected from the first capping layer and the second capping layer may each independently include a compound represented by Formula 201, a compound represented by Formula 202, or any combination thereof.

In one or more embodiments, at least one selected from the first capping layer and the second capping layer may each independently include one selected from Compounds HT28 to HT33, one selected from Compounds CP1 to CP6, β-NPB, or any combination thereof:

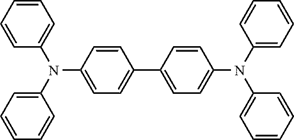 [SMILES: c1ccc(N(c2ccccc2)c2ccc(-c3ccc(N(c4ccccc4)c4ccccc4)cc3)cc2)cc1]

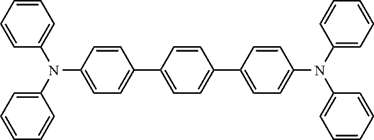 [SMILES: c1ccc(N(c2ccccc2)c2ccc(-c3ccc(-c4ccc(N(c5ccccc5)c5ccccc5)cc4)cc3)cc2)cc1]

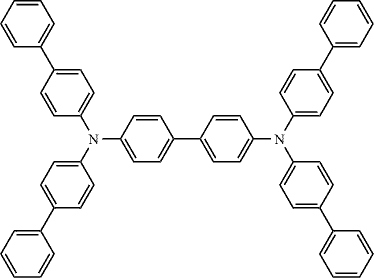 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

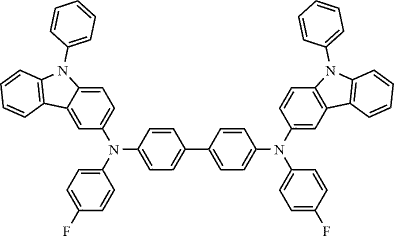 [SMILES: Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

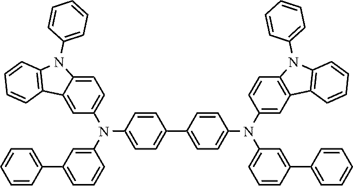 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

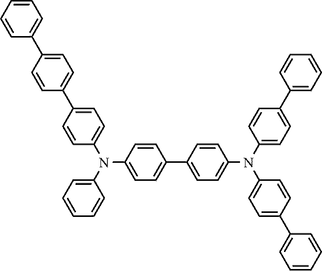 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

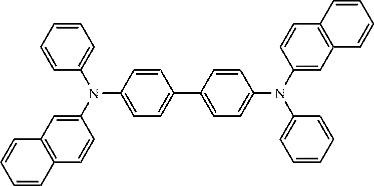 [SMILES: c1ccc(N(c2ccc(-c3ccc(N(c4ccccc4)c4ccc5ccccc5c4)cc3)cc2)c2ccc3ccccc3c2)cc1]

### Electronic Apparatus

The light-emitting device may be included in various suitable electronic apparatuses. For example, the electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be in at least one traveling direction of light emitted from the light-emitting device. For example, the light emitted from the light-emitting device may be blue light or white light. Additional details of the light-emitting device may be the same as described above. In an embodiment, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be located among the subpixel areas to define each of the subpixel areas.

The color filter may further include a plurality of color filter areas and light-shielding patterns located among the color filter areas, and the color conversion layer may further include a plurality of color conversion areas and light-shielding patterns located among the color conversion areas.

The color filter areas (or the color conversion areas) may include a first area emitting first color light, a second area emitting second color light, and/or a third area emitting third color light, and the first color light, the second color light, and/or the third color light may have different maximum emission wavelengths from one another. For example, the first color light may be red light, the second color light may be green light, and the third color light may be blue light. For example, the color filter areas (or the color conversion areas) may include quantum dots. In more detail, the first area may include red quantum dots, the second area may include green quantum dots, and the third area may not include a quantum dot. Additional details of the quantum dots may be the same as described in the present specification. The first area, the second area, and/or the third area may each further include a scatterer (e.g., a light scatterer).

For example, the light-emitting device may emit a first light, the first area may absorb the first light to emit a first-first color light, the second area may absorb the first light to emit a second-first color light, and the third area may absorb the first light to emit a third-first color light. In this regard, the first-first color light, the second-first color light, and the third-first color light may have different maximum emission wavelengths from one another. In more detail, the first light may be blue light, the first-first color light may be red light, the second-first color light may be green light, and the third-first color light may be blue light.

The electronic apparatus may further include a thin-film transistor, in addition to the light-emitting device as described above. The thin-film transistor may include a source electrode, a drain electrode, and an activation layer, wherein one selected from the source electrode and the drain electrode may be electrically connected to one selected from the first electrode and the second electrode of the light-emitting device.

The thin-film transistor may further include a gate electrode, a gate insulating film, and the like.

The activation layer may include crystalline silicon, amorphous silicon, an organic semiconductor, an oxide semiconductor, and/or the like.

The electronic apparatus may further include a sealing portion for sealing the light-emitting device. The sealing portion may be between the color conversion layer and/or color filter and the light-emitting device. The sealing portion allows light from the light-emitting device to be extracted to the outside, and concurrently (e.g., simultaneously) prevents or reduces penetration of ambient air and/or moisture into the light-emitting device. The sealing portion may be a sealing substrate including a transparent glass substrate and/or a plastic substrate. The sealing portion may be a thin-film encapsulation layer including at least one of an organic layer and/or an inorganic layer. When the sealing portion is a thin-film encapsulation layer, the electronic apparatus may be flexible.

Various suitable functional layers may be additionally on the sealing portion, in addition to the color filter and/or the color conversion layer, according to the use of the electronic apparatus. Examples of the functional layers may include a touch screen layer, a polarizing layer, and the like. The touch screen layer may be a pressure-sensitive touch screen layer, a capacitive touch screen layer, and/or an infrared touch screen layer. The authentication apparatus may be, for example, a biometric authentication apparatus that authenticates an individual by using biometric information of a living body (for example, fingertips, pupils, and/or the like).

The authentication apparatus may further include, in addition to the light-emitting device as described above, a biometric information collector.

The electronic apparatus may be applied to various suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, and/or endoscope displays), fish finders, various suitable measuring instruments, meters (for example, meters for a vehicle, an aircraft, and/or a vessel), projectors, and/or the like.

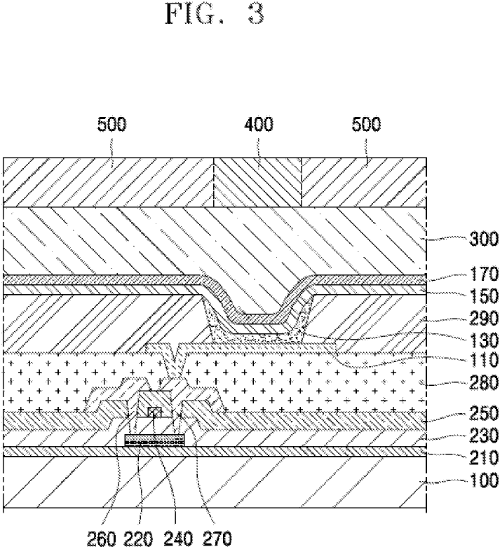

### Description of FIGS. 2 and 3

FIG. 2 is a cross-sectional view of an electronic apparatus according to an embodiment.

The electronic apparatus of FIG. 2 may include a substrate 100, a thin-film transistor (TFT), a light-emitting device, and an encapsulation portion 300 that seals the light-emitting device.

The substrate 100 may be a flexible substrate, a glass substrate, and/or a metal substrate. A buffer layer 210 may be on the substrate 100. The buffer layer 210 may prevent or reduce penetration of impurities through the substrate 100 and may provide a flat surface on the substrate 100.

A TFT may be on the buffer layer 210. The TFT may include an activation layer 220, a gate electrode 240, a source electrode 260, and a drain electrode 270.

The activation layer 220 may include an inorganic semiconductor, such as silicon and/or polysilicon, an organic semiconductor, and/or an oxide semiconductor, and may include a source region, a drain region, and a channel region.

A gate insulating film 230 for insulating the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be between the gate electrode 240 and the source electrode 260 and between the gate electrode 240 and the drain electrode 270 to provide insulation therebetween.

The source electrode 260 and the drain electrode 270 may be on the interlayer insulating film 250. The interlayer insulating film 250 and the gate insulating film 230 may expose the source region and the drain region of the activation layer 220, and the source electrode 260 and the drain electrode 270 may be in contact (e.g., physical contact) with the exposed portions of the source region and the drain region of the activation layer 220.

The TFT may be electrically connected to a light-emitting device to drive the light-emitting device, and may be covered by a passivation layer 280. The passivation layer 280 may include an inorganic insulating film, an organic insulating film, or any combination thereof. A light-emitting device may be provided on the passivation layer 280. The light-emitting device may include a first electrode 110, an interlayer 130, and a second electrode 150.

The first electrode 110 may be on the passivation layer 280. The passivation layer 280 may expose a portion of the drain electrode 270, not fully covering the drain electrode 270, and the first electrode 110 may be connected to the exposed portion of the drain electrode 270.

A pixel defining layer 290 including an insulating material may be on the first electrode 110. The pixel defining layer 290 may expose a portion of the first electrode 110, and the interlayer 130 may be in the exposed portion of the first electrode 110. The pixel defining layer 290 may be a polyimide organic film and/or a polyacrylic organic film. In some embodiments, at least some layers of the interlayer 130 may extend beyond the upper portion of the pixel defining layer 290 in the form of a common layer.

The second electrode 150 may be on the interlayer 130, and a capping layer 170 may be additionally on the second electrode 150. The capping layer 170 may cover the second electrode 150.

The encapsulation portion 300 may be on the capping layer 170. The encapsulation portion 300 may be on a light-emitting device to protect the light-emitting device from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or any combination thereof; or any combination of the inorganic films and the organic films.

FIG. 3 is a cross-sectional view of an electronic apparatus according to another embodiment.

The electronic apparatus of FIG. 3 is substantially the same as the electronic apparatus of FIG. 2, except that a light-shielding pattern 500 and a functional region 400 are additionally on the encapsulation portion 300. The functional region 400 may be i) a color filter area, ii) a color conversion area, or iii) a combination of the color filter area and the color conversion area. In an embodiment, the light-emitting device included in the electronic apparatus of FIG. 3 may be a tandem light-emitting device.

### Manufacturing Method

Respective layers included in the hole transport region, the emission layer, and respective layers included in the electron transport region may be formed in a certain region by using one or more suitable methods selected from vacuum deposition, spin coating, casting, Langmuir-Blodgett (LB) deposition, ink-jet printing, laser-printing, laser-induced thermal imaging, and the like.

When layers constituting the hole transport region, the emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

When layers constituting the hole transport region, the emission layer, and layers constituting the electron transport region are formed by spin coating, the spin coating may be performed at a coating speed of about 2,000 rpm to about 5,000 rpm and at a heat treatment temperature of about 80° C. to about 200° C. by taking into account a material to be included in a layer to be formed and the structure of a layer to be formed.

### Definition of Terms

The term “C₃-C₆₀ carbocyclic group,” as used herein, refers to a cyclic group consisting of carbon only as a ring-forming atom and having 3 to 60 carbon atoms, and the term “C₁-C₆₀ heterocyclic group,” as used herein, refers to a cyclic group that has 1 to 60 carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed together with each other. For example, the C₁-C₆₀ heterocyclic group may have 3 to 61 ring-forming atoms.

The term “cyclic group,” as used herein, may include both the C₃-C₆₀ carbocyclic group and the C₁-C₆₀ heterocyclic group.

The term “π electron-rich C₃-C₆₀ cyclic group,” as used herein, refers to a cyclic group that has 3 to 60 carbon atoms and does not include *—N═*’ as a ring-forming moiety, and the term “π electron-deficient nitrogen-containing C₁-C₆₀ cyclic group,” as used herein, refers to a heterocyclic group that has 1 to 60 carbon atoms and includes *—N═*’ as a ring-forming moiety.

For example,
the C₃-C₆₀ carbocyclic group may be i) a T1 group or ii) a condensed cyclic group in which at least two T1 groups are condensed together with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) a T2 group, ii) a condensed cyclic group in which at least two T2 groups are condensed together with each other, or iii) a condensed cyclic group in which at least one T2 group and at least one T1 group are condensed together with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, or the like),
the π electron-rich C₃-C₆₀ cyclic group may be i) a T1 group, ii) a condensed cyclic group in which at least two T1 groups are condensed together with each other, iii) a T3 group, iv) a condensed cyclic group in which at least two T3 groups are condensed together with each other, or v) a condensed cyclic group in which at least one T3 group and at least one T1 group are condensed together with each other (for example, the C₃-C₆₀ carbocyclic group, a 1 H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, or the like),
the π electron-deficient nitrogen-containing C₁-C₆₀ cyclic group may be i) a T4 group, ii) a condensed cyclic group in which at least two T4 groups are condensed together with each other, iii) a condensed cyclic group in which at least one T4 group and at least one T1 group are condensed together with each other, iv) a condensed cyclic group in which at least one T4 group and at least one T3 group are condensed together with each other, or v) a condensed cyclic group in which at least one T4 group, at least one T1 group, and at least one T3 group are condensed together with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, or the like),
the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,
the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the T3 group may be a furan group, a thiophene group, a 1H-pyrrole group, a silole group, or a borole group, and
the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group”, “C₃-C₆₀ carbocyclic group”, “C₁-C₆₀ heterocyclic group”, “π electron-rich C₃-C₆₀ cyclic group”, or “π electron-deficient nitrogen-containing C₁-C₆₀ cyclic group,” as used herein, may be a group condensed together with any suitable cyclic group, a monovalent group, or a polyvalent group (for example, a divalent group, a trivalent group, a quadrivalent group, and/or the like), depending on the structure of a formula to which the term is applied. For example, the “benzene group” may be a benzo group, a phenyl group, a phenylene group, or the like, which may be easily understood by one of ordinary skill in the art according to the structure of a formula including the “benzene group.”

Examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of the divalent C₃-C₆₀ carbocyclic group and the divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group,” as used herein, refers to a linear or branched aliphatic hydrocarbon monovalent group that has 1 to 60 carbon atoms, and examples thereof include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, and the like. The term “C₁-C₆₀ alkylene group,” as used herein, refers to a divalent group having substantially the same structure as the C₁-C₆₀ alkyl group.

The term “C₂-C₆₀ alkenyl group,” as used herein, refers to a monovalent hydrocarbon group having at least one carbon-carbon double bond at a main chain (e.g., in the middle) or at a terminal end (e.g., the terminus) of the C₂-C₆₀ alkyl group, and examples thereof include an ethenyl group, a propenyl group, a butenyl group, and the like. The term “C₂-C₆₀ alkenylene group,” as used herein, refers to a divalent group having substantially the same structure as the C₂-C₆₀ alkenyl group.

The term “C₂-C₆₀ alkynyl group,” as used herein, refers to a monovalent hydrocarbon group having at least one carbon-carbon triple bond at a main chain (e.g., in the middle) or at a terminal end (e.g., the terminus) of the C₂-C₆₀ alkyl group, and examples thereof include an ethynyl group, a propynyl group, and the like. The term “C₂-C₆₀ alkynylene group,” as used herein, refers to a divalent group having substantially the same structure as the C₂-C₆₀ alkynyl group.

The term “C₁-C₆₀ alkoxy group,” as used herein, refers to a monovalent group represented by -OA₁₀₁ (wherein A₁₀₁ is the C₁-C₆₀ alkyl group), and examples thereof include a methoxy group, an ethoxy group, an isopropyloxy group, and the like.

The term “C₃-C₁₀ cycloalkyl group,” as used herein, refers to a monovalent saturated hydrocarbon cyclic group having 3 to 10 carbon atoms, and examples thereof include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and the like. The term “C₃-C₁₀ cycloalkylene group,” as used herein, refers to a divalent group having substantially the same structure as the C₃-C₁₀ cycloalkyl group.

The term “C₁-C₁₀ heterocycloalkyl group,” as used herein, refers to a monovalent cyclic group of 1 to 10 carbon atoms, further including, in addition to carbon atoms, at least one heteroatom, as ring-forming atoms, and examples thereof include a 1,2,3,4-oxatriazolidinyl group, a tetrahydrofuranyl group, and a tetrahydrothiophenyl group. The term “C₁-C₁₀ heterocycloalkylene group,” as used herein, refers to a divalent group having substantially the same structure as the C₁-C₁₀ heterocycloalkyl group.

The term “C₃-C₁₀ cycloalkenyl group,” as used herein, refers to a monovalent cyclic group that has 3 to 10 carbon atoms and at least one carbon-carbon double bond in the ring thereof and no aromaticity (e.g., is not aromatic), and examples thereof include a cyclopentenyl group, a cyclohexenyl group, a cycloheptenyl group, and the like. The term “C₃-C₁₀ cycloalkenylene group,” as used herein, refers to a divalent group having substantially the same structure as the C₃-C₁₀ cycloalkenyl group.

The term “C₁-C₁₀ heterocycloalkenyl group,” as used herein, refers to a monovalent cyclic group of 1 to 10 carbon atoms, further including, in addition to carbon atoms, at least one heteroatom, as ring-forming atoms, and having at least one carbon-carbon double bond in the cyclic structure thereof. Examples of the C₁-C₁₀ heterocycloalkenyl group include a 4,5-dihydro-1,2,3,4-oxatriazolyl group, a 2,3-dihydrofuranyl group, a 2,3-dihydrothiophenyl group, and the like. The term “C₁-C₁₀ heterocycloalkenylene group,” as used herein, refers to a divalent group having substantially the same structure as the C₁-C₁₀ heterocycloalkenyl group.

The term “C₆-C₆₀ aryl group,” as used herein, refers to a monovalent group having a carbocyclic aromatic system of 6 to 60 carbon atoms, and the term “C₆-C₆₀ arylene group,” as used herein, refers to a divalent group having a carbocyclic aromatic system of 6 to 60 carbon atoms. Examples of the C₆-C₆₀ aryl group include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and the like. When the C₆-C₆₀ aryl group and the C₆-C₆₀ arylene group each include two or more rings, the two or more rings may be condensed together with each other.

The term “C₁-C₆₀ heteroaryl group,” as used herein, refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group,” as used herein, refers to a divalent group having a heterocyclic aromatic system of 1 to 60 carbon atoms, further including, in addition to carbon atoms, at least one heteroatom, as ring-forming atoms. Examples of the C₁-C₆₀ heteroaryl group include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and the like. When the C₁-C₆₀ heteroaryl group and the C₁-C₆₀ heteroarylene group each include two or more rings, the two or more rings may be condensed together with each other.

The term “monovalent non-aromatic condensed polycyclic group,” as used herein, refers to a monovalent group (for example, having 8 to 60 carbon atoms) having two or more rings condensed to each other, only carbon atoms as ring-forming atoms, and no aromaticity in its entire molecular structure (e.g., the entire molecular structure is not aromatic when considered as a whole). Examples of the monovalent non-aromatic condensed polycyclic group include an indenyl group, a fluorenyl group, a spiro-bifluorenyl group, a benzofluorenyl group, an indenophenanthrenyl group, an indenoanthracenyl group, and the like. The term “divalent non-aromatic condensed polycyclic group,” as used herein, refers to a divalent group having substantially the same structure as the monovalent non-aromatic condensed polycyclic group.

The term “monovalent non-aromatic condensed heteropolycyclic group,” as used herein, refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure (e.g., the entire molecular structure is not aromatic when considered as a whole). Examples of the monovalent non-aromatic condensed heteropolycyclic group include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and the like. The term “divalent non-aromatic condensed heteropolycyclic group,” as used herein, refers to a divalent group having substantially the same structure as the monovalent non-aromatic condensed heteropolycyclic group.

The term “C₆-C₆₀ aryloxy group,” as used herein, refers to -OA₁₀₂ (wherein A₁₀₂ is the C₆-C₆₀ aryl group), and the term “C₆-C₆₀ arylthio group,” as used herein, refers to -SA₁₀₃ (wherein A₁₀₃ is the C₆-C₆₀ aryl group).

The term “C₇-C₆₀ aryl alkyl group,” as used herein, refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroaryl alkyl group,” as used herein, refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ,” as used herein, refers to:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, -Si(Q₁₁)(Q₁₂)(Q₁₃), -N(Q₁₁)(Q₁₂), -B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, -Si(Q₂₁)(Q₂₂)(Q₂₃), -N(Q₂₁)(Q₂₂), -B(Q₂₁)(Q₂₂), —C(═O)(Q₂₁), —S(═O)₂(Q₂₁), —P(═O)(Q₂₁)(Q₂₂), or any combination thereof; or
-Si(Q₃₁)(Q₃₂)(Q₃₃), -N(Q₃₁)(Q₃₂), -B(Q₃₁)(Q₃₂), —C(═O)(Q₃₁), —S(═O)₂(Q₃₁), or —P(═O)(Q₃₁)(Q₃₂).
Q₁ to Q₃, Q₁₁ to Q₁₃, Q₂₁ to Q₂₃, and Q₃₁ to Q₃₃ used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom,” as used herein, refers to any atom other than a carbon atom. Examples of the heteroatom include O, S, N, P, Si, B, Ge, Se, or any combination thereof.

The term “the third-row transition metal,” as used herein, includes hafnium (Hf), tantalum (Ta), tungsten (W), rhenium (Re), osmium (Os), iridium (Ir), platinum (Pt), gold (Au), and the like.

The term “Ph”, as used herein, refers to a phenyl group, the term “Me,” as used herein. refers to a methyl group, the term “Et,” as used herein, refers to an ethyl group, the term “ter-Bu” or “Bu^{t},” as used herein, refers to a tert-butyl group, and the term “OMe,” as used herein, refers to a methoxy group.

The term “biphenyl group,” as used herein, refers to “a phenyl group substituted with a phenyl group”. In other words, the “biphenyl group” is a substituted phenyl group having a C₆-C₆₀ aryl group as a substituent.

The term “terphenyl group,” as used herein, refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

The maximum number of carbon atoms in this substituent definition section is an example only. For example, the maximum carbon number of 60 in the C₁-C₆₀ alkyl group is an example, and the definition of the alkyl group equally applies to a C₁-C₂₀ alkyl group. The same applies to other cases.

* and *’, as used herein, unless defined otherwise, each refer to a binding site to a neighboring atom in a corresponding formula.

Hereinafter, compounds according to embodiments and light-emitting devices according to embodiments will be described in more detail with reference to the following Synthesis Examples and Examples. The wording “B was used instead of A” used in describing Synthesis Examples means that an identical molar equivalent of B was used in place of A.

### EXAMPLES

### Synthesis Example: Synthesis of Compounds

### Synthesis Example 1. Synthesis of Compound 1

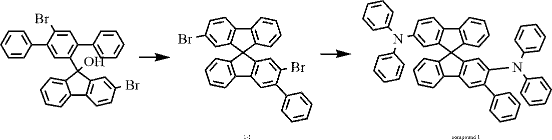

### Synthesis of Intermediate 1-1

5.71 g (10 mmol) of 2-bromo-9-(5′-bromo-[1,1′:4′,1″-terphenyl]-2′-yl)-9H-fluoren-9-ol and 5 mL of trifluoromethanesulfonic acid were dissolved in 100 mL of dichloromethane, and then stirred at room temperature for 2 hours. 40 mL of water was added to the resultant reaction solution, and the resultant mixture was extracted three times with 50 mL of ethyl ether. The collected ethyl ether was dried with MgSO₄ and the residue obtained by evaporating the solvent therefrom was separated and purified by silica gel column chromatography to obtain 4.40 g (yield: 80 %) of Intermediate 1-1. Intermediate 1-1 was identified by LC-MS. C31H18Br2 M+: 550.2

### Synthesis of Compound 1

4.40 g (8.0 mmol) of Intermediate 1-1, 2.70 g (16 mmol) of diphenyl amine, 1.60 g (0.8 mmol) of P(t-Bu)₃, 0.36 g (0.4 mmol) of tris(dibenzylideneacetone)dipalladium (0) (Pd₂dba₃), and 2.3 g (24 mmol) of sodium tert-butoxide were dissolved in 200 mL of toluene, and then stirred at 80° C. for 3 hours. After the resultant reaction solution was cooled to room temperature, 60 mL of water was added thereto, and the resultant mixture was extracted three times with 80 mL of ethyl ether. The collected ethyl ether was dried with MgSO₄ and the residue obtained by evaporating the solvent therefrom was separated and purified by silica gel column chromatography to obtain 3.26 g (yield: 56 %) of Compound 1. Compound 1 was identified by high-resolution mass spectrometry (HRMS). (yield: 56 %, HRMS (EI): calcd.: 726.3035; found: 726.3037.)

### Synthesis Example 2. Synthesis of Compound 3

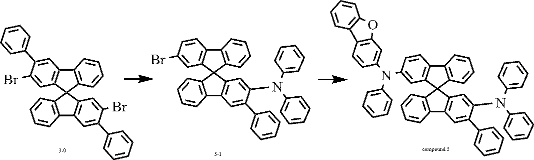

### Synthesis of Intermediate 3-1

0.93 mg (5.5 mmol) of diphenylamine, 3.69 g (6.7 mmol) of Intermediate 3-0, 0.31 g (0.55 mmol) of 1,1′-bis(diphenylphosphino)ferrocene (DPPF), 0.25 g (0.28 mmol) of Pd₂dba₃, and 1.6 g (16.6 mmol) of sodium tert-butoxide were dissolved in 100 mL of toluene, and then stirred at 80° C. for 3 hours. After the resultant reaction solution was cooled to room temperature, 30 mL of water was added thereto, and the resultant mixture was extracted three times with 40 mL of ethyl ether. The collected ethyl ether was dried with MgSO₄ and the residue obtained by evaporating the solvent therefrom was separated and purified by silica gel column chromatography to obtain 0.98 g (yield: 28 %) of Intermediate 3-1. Intermediate 3-1 was identified by LC-MS. C43H28BrN M+: 638.6

### Synthesis of Compound 3

11.8 g (18.5 mmol) of Intermediate 3-1, 6.2 g (24 mmol) of N-phenyldibenzo[b,d]furan-3-amine, 0.85 g (0.93 mmol) of Pd₂dba₃, 0.40 g (1.85 mmol) of P(t-Bu)₃, and 5.4 g (55 mmol) of sodium tert-butoxide were dissolved in 120 mL of toluene, and then stirred at 80° C. for 3 hours. After the resultant reaction solution was cooled to room temperature, 40 mL of water was added thereto, and the resultant mixture was extracted three times with 50 mL of ethyl ether. The collected ethyl ether was dried with MgSO₄ and the residue obtained by evaporating the solvent therefrom was separated and purified by silica gel column chromatography to obtain 9.1 g (yield: 60 %) of Compound 3. Compound 3 was identified by HRMS. (yield: 60 %, HRMS (EI): calcd.: 816.3141; found: 816.3143.)

### Synthesis Example 3. Synthesis of Compound 11

Compound 11 was synthesized in substantially the same manner as used to synthesize Compound 3, except that 9,9-dimethyl-N-phenyl-9H-fluoren-2-amine was used instead of N-phenyldibenzo[b,d]furan-3-amine. Compound 11 was identified by HRMS. (HRMS (EI): calcd.: 842.3661; found: 842.3663.)

### Synthesis Example 4. Synthesis of Compound 31

Compound 31 was synthesized in substantially the same manner as used to synthesize Compound 3, except that N-phenyl-[1,1′:3′,1″-terphenyl]-2′-amine was used instead of N-phenyldibenzo[b,d]furan-3-amine. Compound 31 was identified by HRMS. (HRMS (EI): calcd.: 878.3661; found: 878.3664.)

### Synthesis Example 5. Synthesis of Compound 42

Compound 42 was synthesized in substantially the same manner as used to synthesize Compound 11, except that 2,2′-dibromo-3,3′-diphenyl-9,9′-spirobi[fluorene] was used instead of 2,2′-dibromo-3-phenyl-9,9′-spirobi[fluorene]. Compound 42 was identified by HRMS. (HRMS (EI): calcd.: 918.3974; found: 918.3976.)

### Synthesis Example 6. Synthesis of Compound 88

Compound 88 was synthesized in substantially the same manner as used to synthesize Compound 11, except that 2,2′-dibromo-3,3′-diphenyl-9,9′-spirobi[fluorene] was used instead of 2,2′-dibromo-3-phenyl-9,9′-spirobi[fluorene]. Compound 88 was identified by HRMS. (HRMS (EI): calcd.: 994.4287; found: 994.4289.)

### Synthesis Example 7. Synthesis of Compound 109

Compound 109 was synthesized in substantially the same manner as used to synthesize Compound 11, except that 2,2′-dibromo-3-(4-cyclohexylphenyl)-3′-phenyl-9,9′-spirobi[fluorene] was used instead of 2,2′-dibromo-3-phenyl-9,9′-spirobi[fluorene]. Compound 109 was identified by HRMS. (HRMS (EI): calcd.: 1000.4756; found: 1000.4759.)

### Synthesis Example 8. Synthesis of Compound 118

Compound 118 was synthesized in substantially the same manner as used to synthesize Compound 11, except that 3-(4-(bicyclo[2.2.1]heptan-2-yl)phenyl)-2,2′-dibromo-3′-phenyl-9,9′-spirobi[fluorene] was used instead of 2,2′-dibromo-3-phenyl-9,9′-spirobi[fluorene]. Compound 118 was identified by HRMS. (HRMS (EI): calcd.: 1012.4756; found: 1012.4759.)

### Manufacture of Light-Emitting Device

### Comparative Example 1

As an anode, a glass substrate with 15 Ωcm² (1,200 Å) ITO thereon, which was manufactured by Corning Inc., was cut to a size of 50 mm×50 mm×0.7 mm, and the glass substrate was sonicated by using isopropyl alcohol and pure water for 5 minutes each, and then ultraviolet (UV) light was irradiated for 30 minutes thereto and ozone was exposed thereto for cleaning. Then, the resultant glass substrate was loaded onto a vacuum deposition apparatus.

2-TNATA was vacuum-deposited on the substrate to form a hole injection layer having a thickness of 600 Å, and then, 4,4′-bis[N-(1-naphthyl)-N-phenylamino]biphenyl (hereinafter, referred to as NPB) as a hole transport compound was vacuum-deposited thereon to form a hole transport layer having a thickness of 300 Å.

9,10-di(naphthalen-2-yl)anthracene (hereinafter, referred to as DNA) as a blue fluorescent host and 4,4′-bis[2-(4-(N,N-diphenylamino)phenyl)vinyl]biphenyl (hereinafter, referred to as DPAVBi) as a blue fluorescent dopant were co-deposited on the hole transport layer at a weight ratio of 98:2 to form an emission layer having a thickness of 300 Å.

Next, Alq₃ was deposited on the emission layer to form an electron transport having a thickness of 300 Å, LiF, which is a halogenated alkali metal, was deposited on the electron transport layer to form an electron injection layer having a thickness of 10 Å, and Al was vacuum-deposited thereon to form a cathode having a thickness of 3,000 Å, thereby completing the manufacture of a light-emitting device.

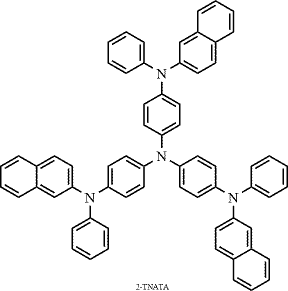

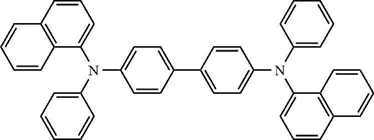 [SMILES: c1ccc(N(c2ccc(-c3ccc(N(c4ccccc4)c4cccc5ccccc45)cc3)cc2)c2cccc3ccccc23)cc1]

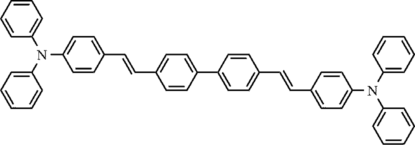 [SMILES: C(=C\c1ccc(N(c2ccccc2)c2ccccc2)cc1)/c1ccc(-c2ccc(/C=C/c3ccc(N(c4ccccc4)c4ccccc4)cc3)cc2)cc1]

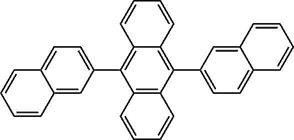 [SMILES: c1ccc2cc(-c3c4ccccc4c(-c4ccc5ccccc5c4)c4ccccc34)ccc2c1]

### Comparative Example 2

A light-emitting device was manufactured in substantially the same manner as in Comparative Example 1, except that Compound 100 was used instead of NPB in forming a hole transport layer.

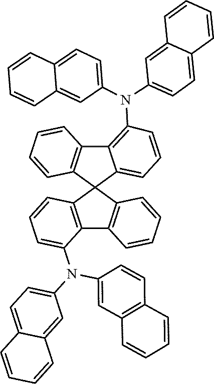 [SMILES: c1ccc2c(c1)-c1c(N(c3ccc4ccccc4c3)c3ccc4ccccc4c3)cccc1C21c2ccccc2-c2c(N(c3ccc4ccccc4c3)c3ccc4ccccc4c3)cccc21]

### Example 1

A light-emitting device was manufactured in substantially the same manner as in Comparative Example 1, except that Compound 1 was used instead of NPB in forming a hole transport layer.

### Example 2

A light-emitting device was manufactured in substantially the same manner as in Comparative Example 1, except that Compound 3 was used instead of NPB in forming a hole transport layer.

### Example 3

A light-emitting device was manufactured in substantially the same manner as in Comparative Example 1, except that Compound 11 was used instead of NPB in forming a hole transport layer.

### Example 4

A light-emitting device was manufactured in substantially the same manner as in Comparative Example 1, except that Compound 31 was used instead of NPB in forming a hole transport layer.

### Example 5

A light-emitting device was manufactured in substantially the same manner as in Comparative Example 1, except that Compound 42 was used instead of NPB in forming a hole transport layer.

### Example 6

A light-emitting device was manufactured in substantially the same manner as in Comparative Example 1, except that Compound 88 was used instead of NPB in forming a hole transport layer.

### Example 7

A light-emitting device was manufactured in substantially the same manner as in Comparative Example 1, except that Compound 109 was used instead of NPB in forming a hole transport layer.

### Example 8

A light-emitting device was manufactured in substantially the same manner as in Comparative Example 1, except that Compound 118 was used instead of NPB in forming a hole transport layer.

The driving voltage, efficiency, and lifespan of each of the light-emitting devices manufactured according to Comparative Example 1 and Examples 1 to 8 are shown in Table 1.

TABLE 1

| | Hole transport layer | Driving voltage (V) | Current density (mA/cm²) | Luminance (cd/m²) | Efficiency (cd/A) | Emission color | Half lifespan (hr @100 mA/cm²) |
|---|---|---|---|---|---|---|---|
| Comparative Example 1 | NPB | 7.01 | 50 | 2645 | 5.29 | Blue | 258 |
| Comparative Example 2 | 100 | 6.05 | 50 | 2865 | 5.73 | Blue | 350 |
| Example 1 | Compound 1 | 5.03 | 50 | 3105 | 6.21 | Blue | 570 |
| Example 2 | Compound 3 | 5.08 | 50 | 3205 | 6.41 | Blue | 600 |
| Example 3 | Compound 11 | 4.98 | 50 | 3100 | 6.20 | Blue | 580 |
| Example 4 | Compound 31 | 5.12 | 50 | 3310 | 6.62 | Blue | 480 |
| Example 5 | Compound 42 | 5.05 | 50 | 3275 | 6.55 | Blue | 530 |
| Example 6 | Compound 88 | 5.10 | 50 | 3090 | 6.18 | Blue | 540 |
| Example 7 | Compound 109 | 4.99 | 50 | 3185 | 6.37 | Blue | 490 |
| Example 8 | Compound 118 | 5.02 | 50 | 3260 | 6.52 | Blue | 520 |

Referring to Table 1, it can be seen that the light-emitting devices of Examples 1 to 8 exhibit excellent results compared to the light-emitting device of Comparative Example 1.

The diamine-based compound represented by Formula 1 according to an embodiment may further include one or more substituents represented by Formulae 2-1 to 2-4. Accordingly, the diamine-based compound represented by Formula 1 may have a more bulky molecular structure. Furthermore, the diamine-based compound may maintain a suitable or optimal intermolecular density.

In addition, amine moieties included in the diamine-based compound represented by Formula 1 may have different electrochemical environments. As a result, energy levels such as HOMO, LUMO, T1, and S1 may be finely adjusted, and hole mobility may be easily controlled.

As a result, the hole mobility and heat resistance may be evenly improved, and an electronic device, for example, an organic light-emitting device, including the diamine-based compound may have a low driving voltage, high efficiency, and a long lifespan.

For example, it can be seen that the compounds of Formula 1 have greater steric hindrance than Compound 100 of Comparative Example 2, and thus, the light-emitting devices of the Examples exhibited better results than the light-emitting device of Comparative Example 2.

As described above, according to the one or more embodiments, a light-emitting device including a compound represented by Formula 1 may exhibit excellent efficiency and improved lifespan.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the present disclosure as defined by the following claims, and equivalents thereof.

What is claimed is:

1. A light-emitting device comprising:
a first electrode;
a second electrode facing the first electrode; and
an interlayer between the first electrode and the second electrode and comprising an emission layer,
wherein the interlayer comprises a layer comprising a compound represented by Formula 1: 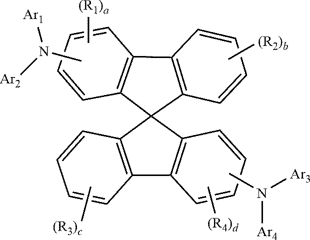
wherein, in Formula 1,
Ar₁ to Ar₄ and R₁ to R₄ are each independently selected from hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₈-C₆₀ monovalent non-aromatic condensed polycyclic group unsubstituted or substituted with at least one R₁₀ₐ, a C₁-C₆₀ monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), and —P(═S)(Q₁)(Q₂),
a and d are each independently an integer from 1 to 3,
b and c are each independently an integer from 1 to 4,
the sum of a and d is an integer of 1 or more, and a case in which each of R₁ and R₄ is hydrogen or deuterium is excluded,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q11)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The light-emitting device of claim 1, wherein the first electrode is an anode,
the second electrode is a cathode, and
the interlayer further comprises an electron transport region between the second electrode and the emission layer and comprising a hole blocking layer, an electron injection layer, an electron transport layer, or any combination thereof.

3. The light-emitting device of claim 1, wherein the first electrode is an anode,
the second electrode is a cathode, and
the interlayer further comprises a hole transport region between the first electrode and the emission layer, the hole transport region comprising a hole transport layer; and an electron blocking layer, a hole injection layer, or any combination thereof.

4. The light-emitting device of claim 1, wherein, in Formula 1, moiety —NAr₁Ar₂ and R₁ are adjacent to each other.

5. The light-emitting device of claim 1, wherein, in Formula 1, moiety —NAr₃Ar₄ and R₄ are adjacent to each other.

6. The light-emitting device of claim 1, wherein Formula 1 is represented by one selected from Formulae 2-1 to 2-4: 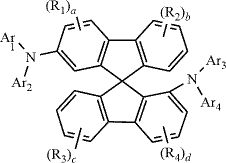 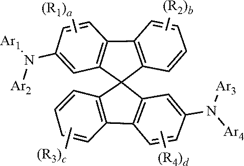 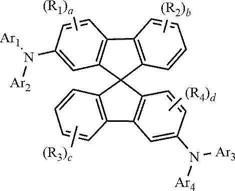 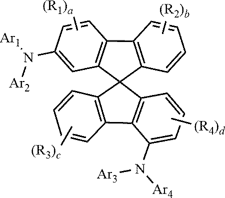
wherein, in Formulae 2-1 to 2-4, R₁ to R₄, a to d, and Ar₁ to Ar₄ are respectively the same as those described with respect to Formula 1.

7. The light-emitting device of claim 1, wherein Formula 1 is represented by one selected from Formulae 3-1 and 3-2: 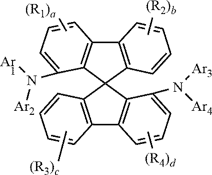 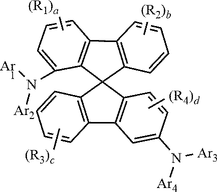
wherein, in Formulae 3-1 and 3-2, R₁ to R₄, a to d, and Ar₁ to Ar₄ are respectively the same as those described with respect to Formula 1.

8. The light-emitting device of claim 1, wherein Formula 1 is represented by one selected from Formulae 4-1 and 4-2: 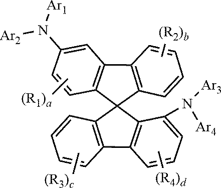 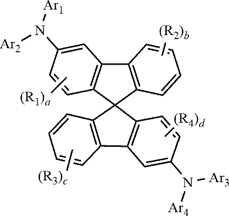
wherein, in Formulae 4-1 and 4-2, R₁ to R₄, a to d, and Ar₁ to Ar₄ are respectively the same as those described with respect to Formula 1.

9. The light-emitting device of claim 1, wherein Formula 1 is represented by one selected from Formulae 5-1 to 5-3: 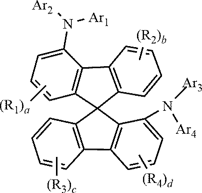 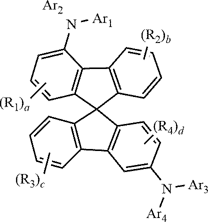 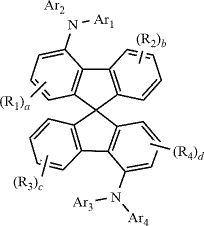
wherein, in Formulae 5-1 to 5-3, R₁ to R₄, a to d, and Ar₁ to Ar₄ are respectively the same as those described with respect to Formula 1.

10. The light-emitting device of claim 1, wherein Ar₁ to Ar₄ are each independently selected from groups represented by Formulae 6-1 to 6-3: 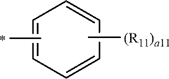 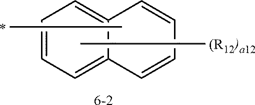 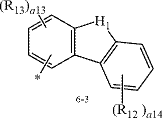
wherein, in Formulae 6-1 to 6-3, H₁ represents O, S, NR₂₁, or CR₂₂R₂₃, and R₁₁ to R₁₄ and R₂₁ to R₂₃ are each independently selected from hydrogen, deuterium, —F, —CI, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), and —P(═O)(Q₁)(Q₂),
a11 is an integer from 1 to 5,
a12 is an integer from 1 to 7,
a13 is an integer from 1 to 3,
a14 is an integer from 1 to 4, and
Q₁ to Q₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

11. The light-emitting device of claim 1, wherein R₁ to R₄ are each independently selected from a C₁-C₆₀ alkyl group and groups represented by Formulae 7-1 to 7-4: 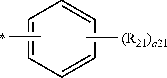 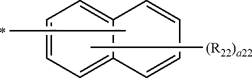 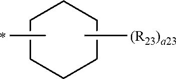 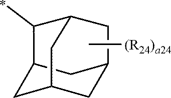
wherein, in Formulae 7-1 to 7-4, R₂₁ to R₂₄ are each independently selected from hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), and —P(═O)(Q₁)(Q₂),
a21 and a23 are each independently an integer from 1 to 5,
a22 is an integer from 1 to 7,
a24 is an integer from 1 to 9, and
Q₁ to Q₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

12. The light-emitting device of claim 1, wherein the compound represented by Formula 1 is selected from the following compounds: 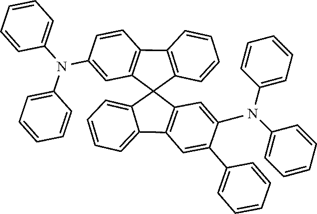 [SMILES: c1ccc(-c2cc3c(cc2N(c2ccccc2)c2ccccc2)C2(c4ccccc4-c4ccc(N(c5ccccc5)c5ccccc5)cc42)c2ccccc2-3)cc1] 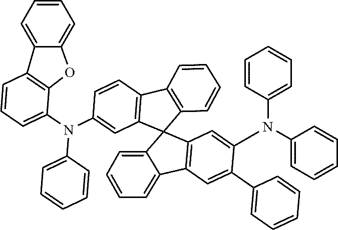 [SMILES: c1ccc(-c2cc3c(cc2N(c2ccccc2)c2ccccc2)C2(c4ccccc4-c4ccc(N(c5ccccc5)c5cccc6c5oc5ccccc56)cc42)c2ccccc2-3)cc1] 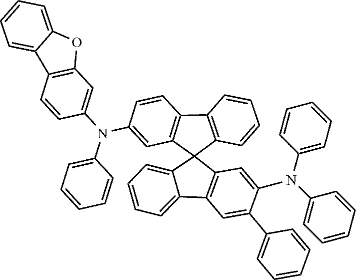 [SMILES: c1ccc(-c2cc3c(cc2N(c2ccccc2)c2ccccc2)C2(c4ccccc4-c4ccc(N(c5ccccc5)c5ccc6c(c5)oc5ccccc56)cc42)c2ccccc2-3)cc1] 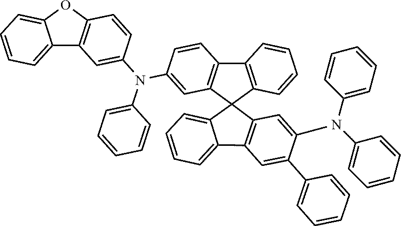 [SMILES: c1ccc(-c2cc3c(cc2N(c2ccccc2)c2ccccc2)C2(c4ccccc4-c4ccc(N(c5ccccc5)c5ccc6oc7ccccc7c6c5)cc42)c2ccccc2-3)cc1] 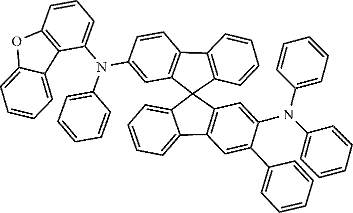 [SMILES: c1ccc(-c2cc3c(cc2N(c2ccccc2)c2ccccc2)C2(c4ccccc4-c4ccc(N(c5ccccc5)c5cccc6oc7ccccc7c56)cc42)c2ccccc2-3)cc1] 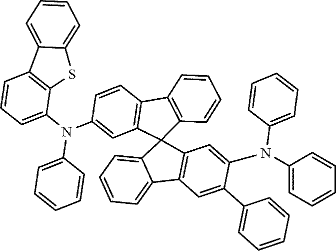 [SMILES: c1ccc(-c2cc3c(cc2N(c2ccccc2)c2ccccc2)C2(c4ccccc4-c4ccc(N(c5ccccc5)c5cccc6c5sc5ccccc56)cc42)c2ccccc2-3)cc1] 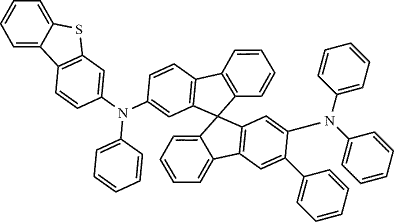 [SMILES: c1ccc(-c2cc3c(cc2N(c2ccccc2)c2ccccc2)C2(c4ccccc4-c4ccc(N(c5ccccc5)c5ccc6c(c5)sc5ccccc56)cc42)c2ccccc2-3)cc1] 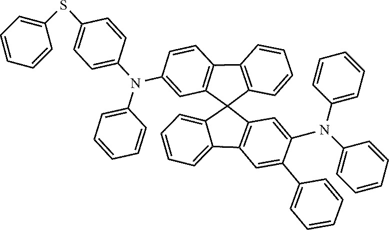 [SMILES: c1ccc(Sc2ccc(N(c3ccccc3)c3ccc4c(c3)C3(c5ccccc5-4)c4ccccc4-c4cc(-c5ccccc5)c(N(c5ccccc5)c5ccccc5)cc43)cc2)cc1] 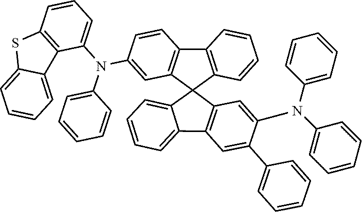 [SMILES: c1ccc(-c2cc3c(cc2N(c2ccccc2)c2ccccc2)C2(c4ccccc4-c4ccc(N(c5ccccc5)c5cccc6sc7ccccc7c56)cc42)c2ccccc2-3)cc1] 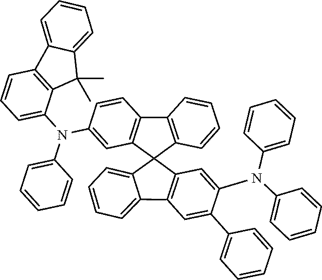 [SMILES: CC1(C)c2ccccc2-c2cccc(N(c3ccccc3)c3ccc4c(c3)C3(c5ccccc5-4)c4ccccc4-c4cc(-c5ccccc5)c(N(c5ccccc5)c5ccccc5)cc43)c21] 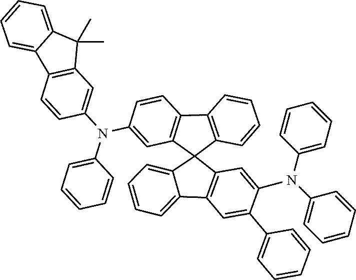 [SMILES: CC1(C)c2ccccc2-c2ccc(N(c3ccccc3)c3ccc4c(c3)C3(c5ccccc5-4)c4ccccc4-c4cc(-c5ccccc5)c(N(c5ccccc5)c5ccccc5)cc43)cc21] 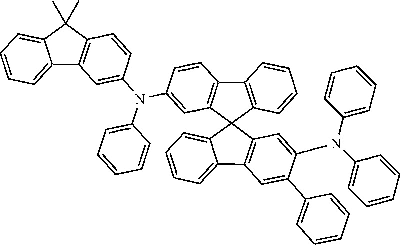 [SMILES: CC1(C)c2ccccc2-c2cc(N(c3ccccc3)c3ccc4c(c3)C3(c5ccccc5-4)c4ccccc4-c4cc(-c5ccccc5)c(N(c5ccccc5)c5ccccc5)cc43)ccc21] 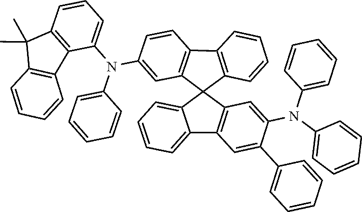 [SMILES: CC1(C)c2ccccc2-c2c(N(c3ccccc3)c3ccc4c(c3)C3(c5ccccc5-4)c4ccccc4-c4cc(-c5ccccc5)c(N(c5ccccc5)c5ccccc5)cc43)cccc21] 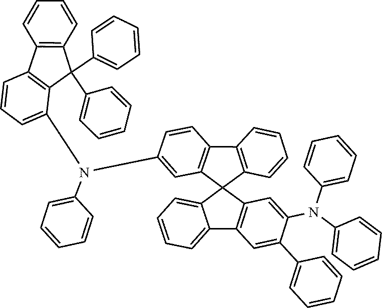 [SMILES: c1ccc(-c2cc3c(cc2N(c2ccccc2)c2ccccc2)C2(c4ccccc4-c4ccc(N(c5ccccc5)c5cccc6c5C(c5ccccc5)(c5ccccc5)c5ccccc5-6)cc42)c2ccccc2-3)cc1] 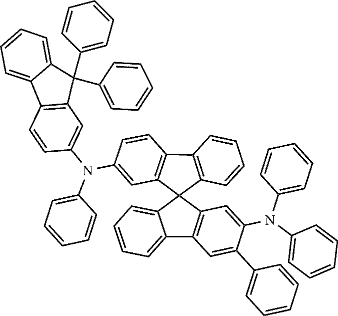 [SMILES: c1ccc(-c2cc3c(cc2N(c2ccccc2)c2ccccc2)C2(c4ccccc4-c4ccc(N(c5ccccc5)c5ccc6c(c5)C(c5ccccc5)(c5ccccc5)c5ccccc5-6)cc42)c2ccccc2-3)cc1] 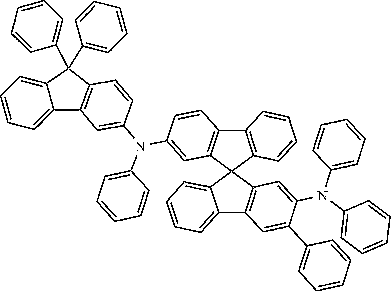 [SMILES: c1ccc(-c2cc3c(cc2N(c2ccccc2)c2ccccc2)C2(c4ccccc4-c4ccc(N(c5ccccc5)c5ccc6c(c5)-c5ccccc5C6(c5ccccc5)c5ccccc5)cc42)c2ccccc2-3)cc1] 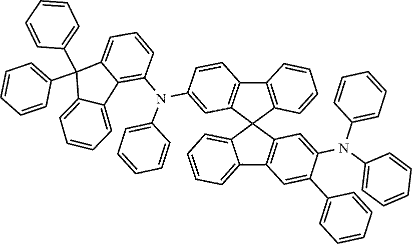 [SMILES: c1ccc(-c2cc3c(cc2N(c2ccccc2)c2ccccc2)C2(c4ccccc4-c4ccc(N(c5ccccc5)c5cccc6c5-c5ccccc5C6(c5ccccc5)c5ccccc5)cc42)c2ccccc2-3)cc1] 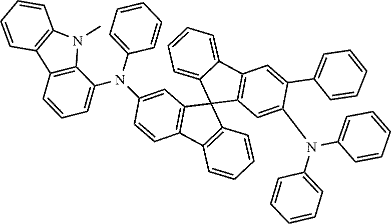 [SMILES: Cn1c2ccccc2c2cccc(N(c3ccccc3)c3ccc4c(c3)C3(c5ccccc5-4)c4ccccc4-c4cc(-c5ccccc5)c(N(c5ccccc5)c5ccccc5)cc43)c21] 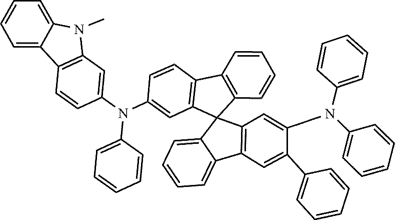 [SMILES: Cn1c2ccccc2c2ccc(N(c3ccccc3)c3ccc4c(c3)C3(c5ccccc5-4)c4ccccc4-c4cc(-c5ccccc5)c(N(c5ccccc5)c5ccccc5)cc43)cc21] 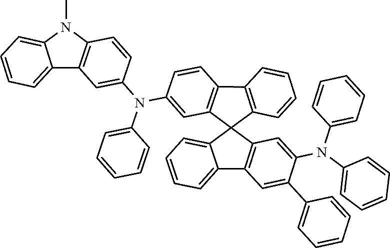 [SMILES: Cn1c2ccccc2c2cc(N(c3ccccc3)c3ccc4c(c3)C3(c5ccccc5-4)c4ccccc4-c4cc(-c5ccccc5)c(N(c5ccccc5)c5ccccc5)cc43)ccc21] 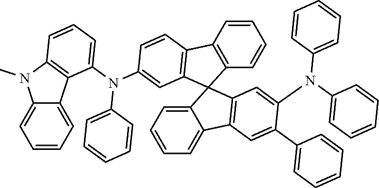 [SMILES: Cn1c2ccccc2c2c(N(c3ccccc3)c3ccc4c(c3)C3(c5ccccc5-4)c4ccccc4-c4cc(-c5ccccc5)c(N(c5ccccc5)c5ccccc5)cc43)cccc21] 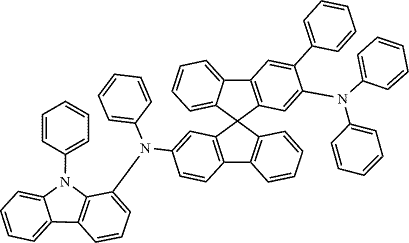 [SMILES: c1ccc(-c2cc3c(cc2N(c2ccccc2)c2ccccc2)C2(c4ccccc4-c4ccc(N(c5ccccc5)c5cccc6c7ccccc7n(-c7ccccc7)c56)cc42)c2ccccc2-3)cc1] 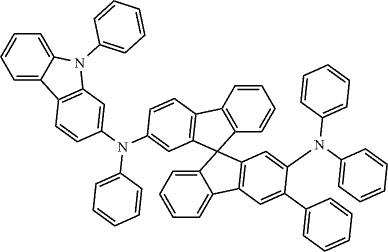 [SMILES: c1ccc(-c2cc3c(cc2N(c2ccccc2)c2ccccc2)C2(c4ccccc4-c4ccc(N(c5ccccc5)c5ccc6c7ccccc7n(-c7ccccc7)c6c5)cc42)c2ccccc2-3)cc1] 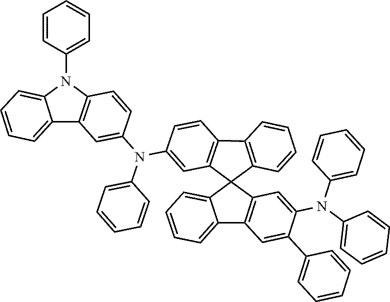 [SMILES: c1ccc(-c2cc3c(cc2N(c2ccccc2)c2ccccc2)C2(c4ccccc4-c4ccc(N(c5ccccc5)c5ccc6c(c5)c5ccccc5n6-c5ccccc5)cc42)c2ccccc2-3)cc1] 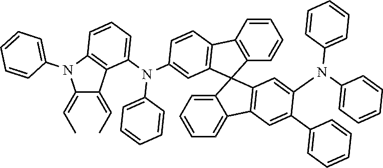 [SMILES: C/C=c1\c(=C/C)n(-c2ccccc2)c2cccc(N(c3ccccc3)c3ccc4c(c3)C3(c5ccccc5-4)c4ccccc4-c4cc(-c5ccccc5)c(N(c5ccccc5)c5ccccc5)cc43)c12] 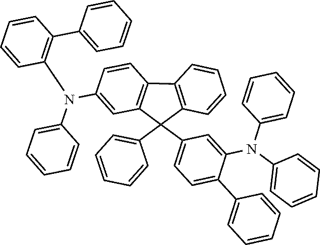 [SMILES: c1ccc(-c2ccccc2N(c2ccccc2)c2ccc3c(c2)C(c2ccccc2)(c2ccc(-c4ccccc4)c(N(c4ccccc4)c4ccccc4)c2)c2ccccc2-3)cc1] 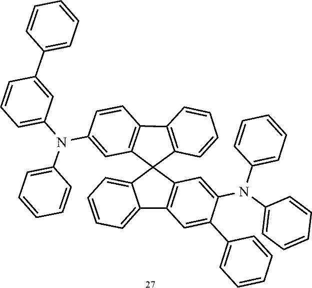 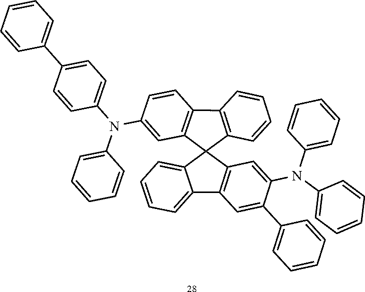 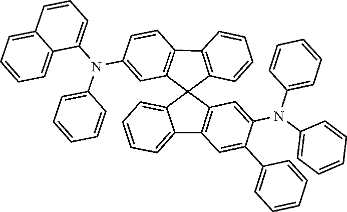 [SMILES: c1ccc(-c2cc3c(cc2N(c2ccccc2)c2ccccc2)C2(c4ccccc4-c4ccc(N(c5ccccc5)c5cccc6ccccc56)cc42)c2ccccc2-3)cc1] 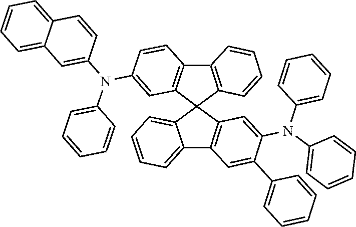 [SMILES: c1ccc(-c2cc3c(cc2N(c2ccccc2)c2ccccc2)C2(c4ccccc4-c4ccc(N(c5ccccc5)c5ccc6ccccc6c5)cc42)c2ccccc2-3)cc1] 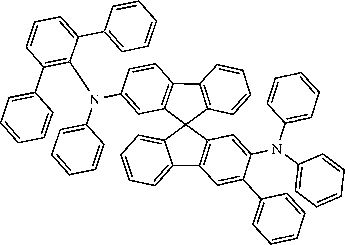 [SMILES: c1ccc(-c2cc3c(cc2N(c2ccccc2)c2ccccc2)C2(c4ccccc4-c4ccc(N(c5ccccc5)c5c(-c6ccccc6)cccc5-c5ccccc5)cc42)c2ccccc2-3)cc1] 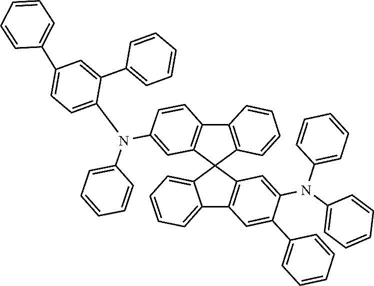 [SMILES: c1ccc(-c2ccc(N(c3ccccc3)c3ccc4c(c3)C3(c5ccccc5-4)c4ccccc4-c4cc(-c5ccccc5)c(N(c5ccccc5)c5ccccc5)cc43)c(-c3ccccc3)c2)cc1] 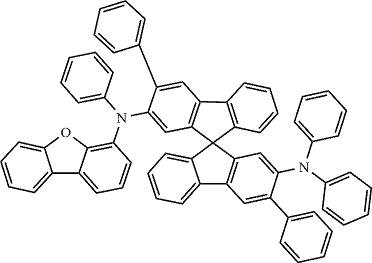 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4cccc5c4oc4ccccc45)cc32)cc1] 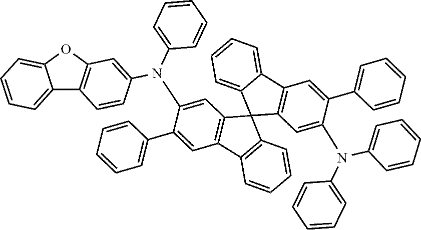 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5c(c4)oc4ccccc45)cc32)cc1] 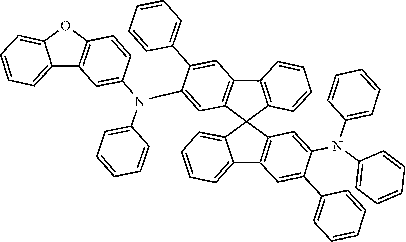 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5oc6ccccc6c5c4)cc32)cc1] 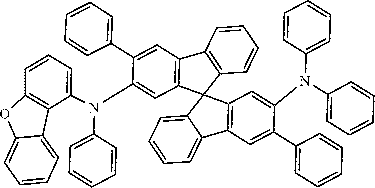 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4cccc5oc6ccccc6c45)cc32)cc1] 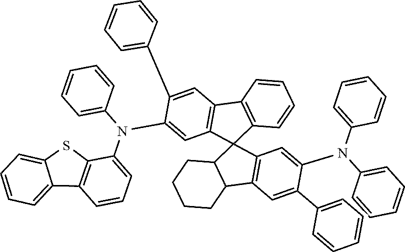 [SMILES: c1ccc(-c2cc3c(cc2N(c2ccccc2)c2ccccc2)C2(c4ccccc4-c4cc(-c5ccccc5)c(N(c5ccccc5)c5cccc6c5sc5ccccc56)cc42)C2CCCCC32)cc1] 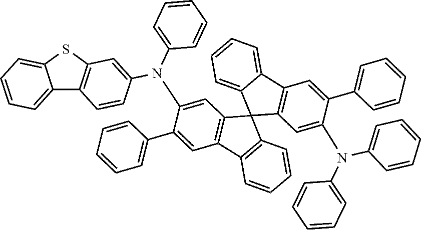 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5c(c4)sc4ccccc45)cc32)cc1] 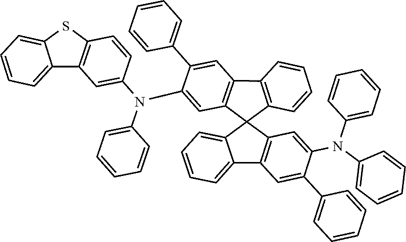 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5sc6ccccc6c5c4)cc32)cc1] 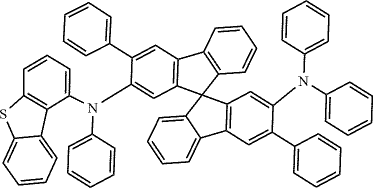 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4cccc5sc6ccccc6c45)cc32)cc1] 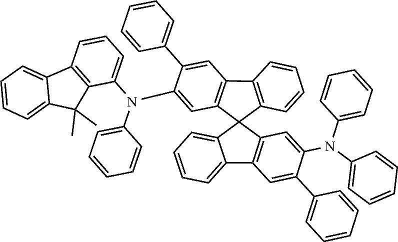 [SMILES: CC1(C)c2ccccc2-c2cccc(N(c3ccccc3)c3cc4c(cc3-c3ccccc3)-c3ccccc3C43c4ccccc4-c4cc(-c5ccccc5)c(N(c5ccccc5)c5ccccc5)cc43)c21] 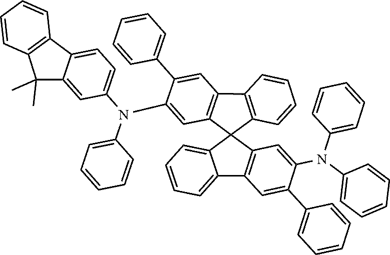 [SMILES: CC1(C)c2ccccc2-c2ccc(N(c3ccccc3)c3cc4c(cc3-c3ccccc3)-c3ccccc3C43c4ccccc4-c4cc(-c5ccccc5)c(N(c5ccccc5)c5ccccc5)cc43)cc21] 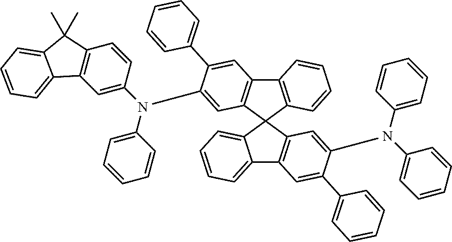 [SMILES: CC1(C)c2ccccc2-c2cc(N(c3ccccc3)c3cc4c(cc3-c3ccccc3)-c3ccccc3C43c4ccccc4-c4cc(-c5ccccc5)c(N(c5ccccc5)c5ccccc5)cc43)ccc21] 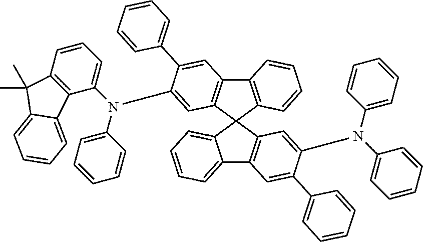 [SMILES: CC1(C)c2ccccc2-c2c(N(c3ccccc3)c3cc4c(cc3-c3ccccc3)-c3ccccc3C43c4ccccc4-c4cc(-c5ccccc5)c(N(c5ccccc5)c5ccccc5)cc43)cccc21] 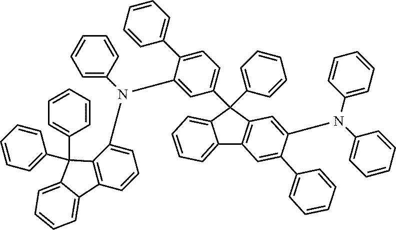 [SMILES: c1ccc(-c2cc3c(cc2N(c2ccccc2)c2ccccc2)C(c2ccccc2)(c2ccc(-c4ccccc4)c(N(c4ccccc4)c4cccc5c4C(c4ccccc4)(c4ccccc4)c4ccccc4-5)c2)c2ccccc2-3)cc1] 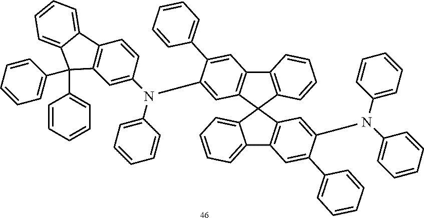 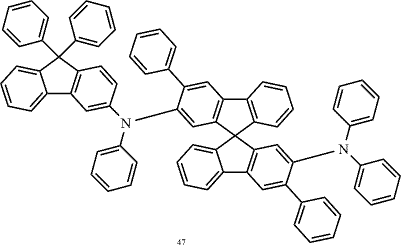 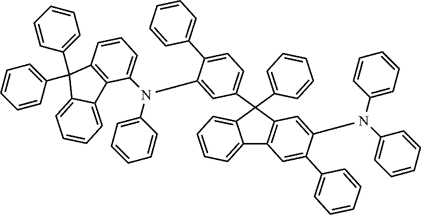 [SMILES: c1ccc(-c2cc3c(cc2N(c2ccccc2)c2ccccc2)C(c2ccccc2)(c2ccc(-c4ccccc4)c(N(c4ccccc4)c4cccc5c4-c4ccccc4C5(c4ccccc4)c4ccccc4)c2)c2ccccc2-3)cc1] 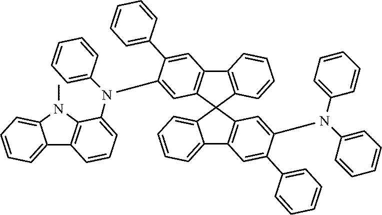 [SMILES: Cn1c2ccccc2c2cccc(N(c3ccccc3)c3cc4c(cc3-c3ccccc3)-c3ccccc3C43c4ccccc4-c4cc(-c5ccccc5)c(N(c5ccccc5)c5ccccc5)cc43)c21] 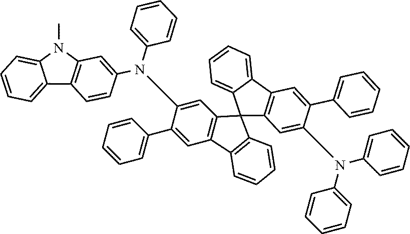 [SMILES: Cn1c2ccccc2c2ccc(N(c3ccccc3)c3cc4c(cc3-c3ccccc3)-c3ccccc3C43c4ccccc4-c4cc(-c5ccccc5)c(N(c5ccccc5)c5ccccc5)cc43)cc21] 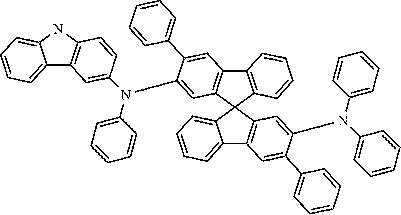 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5[nH]c6ccccc6c5c4)cc32)cc1] 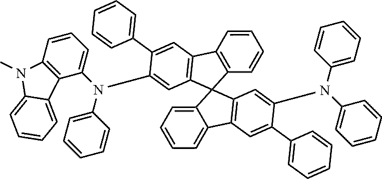 [SMILES: Cn1c2ccccc2c2c(N(c3ccccc3)c3cc4c(cc3-c3ccccc3)-c3ccccc3C43c4ccccc4-c4cc(-c5ccccc5)c(N(c5ccccc5)c5ccccc5)cc43)cccc21] 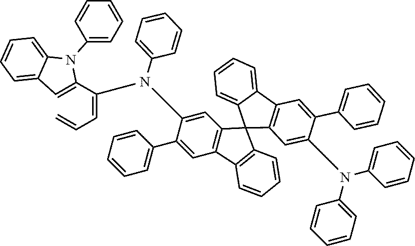 [SMILES: C=C/C=C(\c1cc2ccccc2n1-c1ccccc1)N(c1ccccc1)c1cc2c(cc1-c1ccccc1)-c1ccccc1C21c2ccccc2-c2cc(-c3ccccc3)c(N(c3ccccc3)c3ccccc3)cc21] 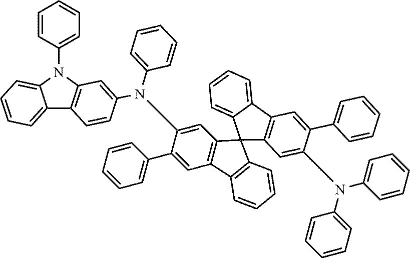 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5c6ccccc6n(-c6ccccc6)c5c4)cc32)cc1] 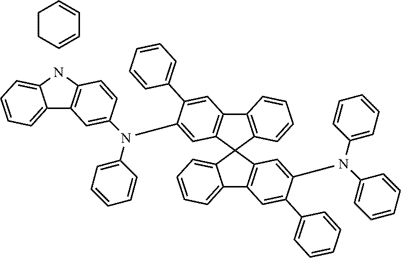 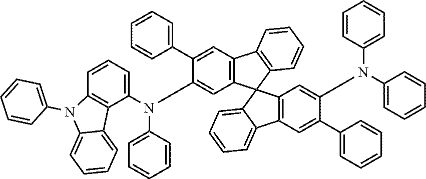 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4cccc5c4c4ccccc4n5-c4ccccc4)cc32)cc1] 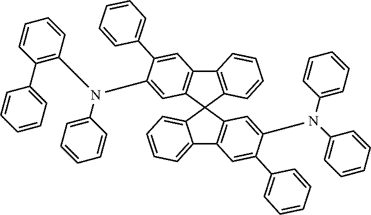 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccccc4-c4ccccc4)cc32)cc1] 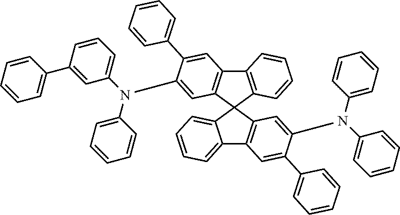 [SMILES: c1ccc(-c2cccc(N(c3ccccc3)c3cc4c(cc3-c3ccccc3)-c3ccccc3C43c4ccccc4-c4cc(-c5ccccc5)c(N(c5ccccc5)c5ccccc5)cc43)c2)cc1] 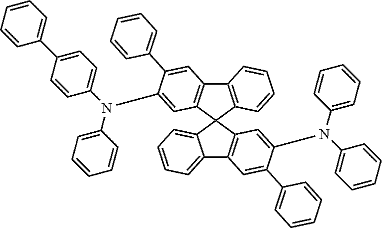 [SMILES: c1ccc(-c2ccc(N(c3ccccc3)c3cc4c(cc3-c3ccccc3)-c3ccccc3C43c4ccccc4-c4cc(-c5ccccc5)c(N(c5ccccc5)c5ccccc5)cc43)cc2)cc1] 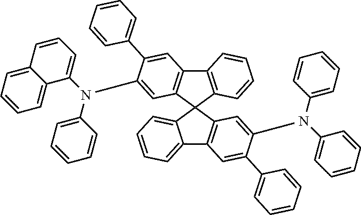 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4cccc5ccccc45)cc32)cc1] 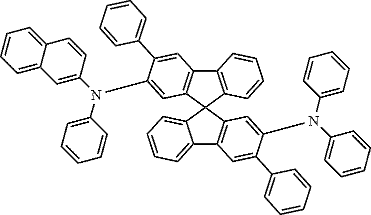 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4ccc5ccccc5c4)cc32)cc1] 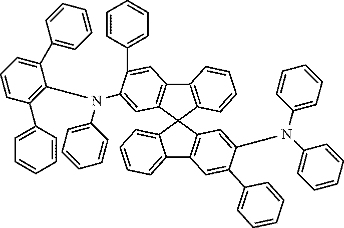 [SMILES: c1ccc(-c2cc3c(cc2N(c2ccccc2)c2ccccc2)C2(c4ccccc4-3)c3ccccc3-c3cc(-c4ccccc4)c(N(c4ccccc4)c4c(-c5ccccc5)cccc4-c4ccccc4)cc32)cc1] 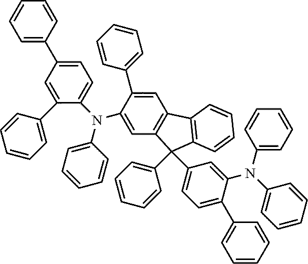 [SMILES: c1ccc(-c2ccc(N(c3ccccc3)c3cc4c(cc3-c3ccccc3)-c3ccccc3C4(c3ccccc3)c3ccc(-c4ccccc4)c(N(c4ccccc4)c4ccccc4)c3)c(-c3ccccc3)c2)cc1] 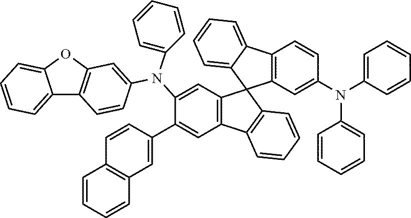 [SMILES: c1ccc(N(c2ccccc2)c2ccc3c(c2)C2(c4ccccc4-3)c3ccccc3-c3cc(-c4ccc5ccccc5c4)c(N(c4ccccc4)c4ccc5c(c4)oc4ccccc45)cc32)cc1] 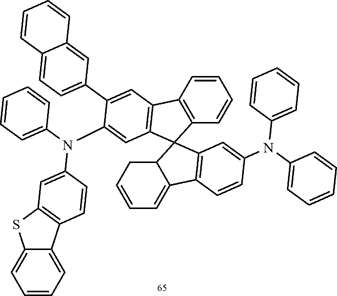 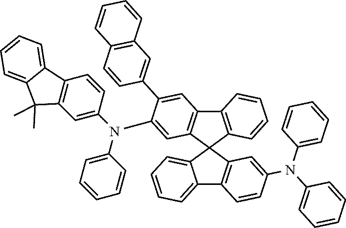 [SMILES: CC1(C)c2ccccc2-c2ccc(N(c3ccccc3)c3cc4c(cc3-c3ccc5ccccc5c3)-c3ccccc3C43c4ccccc4-c4ccc(N(c5ccccc5)c5ccccc5)cc43)cc21] 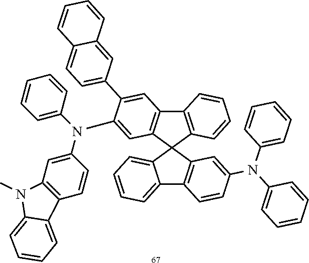 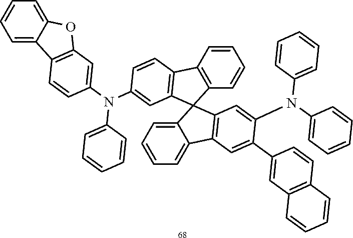 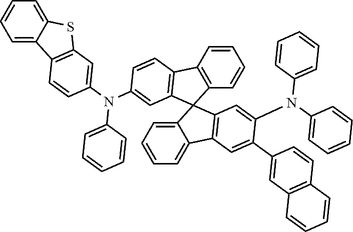 [SMILES: c1ccc(N(c2ccc3c(c2)C2(c4ccccc4-3)c3ccccc3-c3cc(-c4ccc5ccccc5c4)c(N(c4ccccc4)c4ccccc4)cc32)c2ccc3c(c2)sc2ccccc23)cc1] 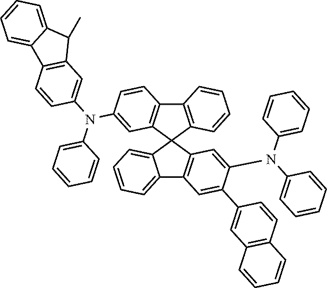 [SMILES: CC1c2ccccc2-c2ccc(N(c3ccccc3)c3ccc4c(c3)C3(c5ccccc5-4)c4ccccc4-c4cc(-c5ccc6ccccc6c5)c(N(c5ccccc5)c5ccccc5)cc43)cc21] 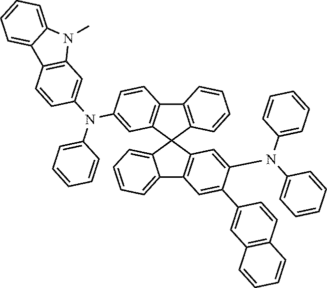 [SMILES: Cn1c2ccccc2c2ccc(N(c3ccccc3)c3ccc4c(c3)C3(c5ccccc5-4)c4ccccc4-c4cc(-c5ccc6ccccc6c5)c(N(c5ccccc5)c5ccccc5)cc43)cc21] 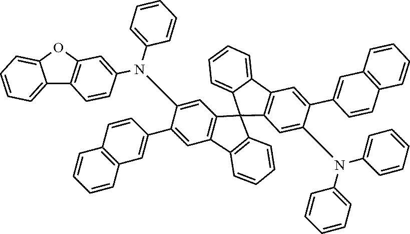 [SMILES: c1ccc(N(c2ccccc2)c2cc3c(cc2-c2ccc4ccccc4c2)-c2ccccc2C32c3ccccc3-c3cc(-c4ccc5ccccc5c4)c(N(c4ccccc4)c4ccc5c(c4)oc4ccccc45)cc32)cc1] 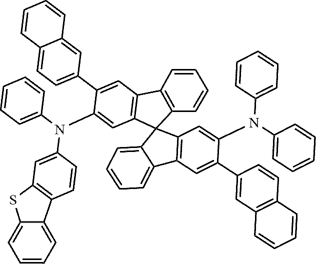 [SMILES: c1ccc(N(c2ccccc2)c2cc3c(cc2-c2ccc4ccccc4c2)-c2ccccc2C32c3ccccc3-c3cc(-c4ccc5ccccc5c4)c(N(c4ccccc4)c4ccc5c(c4)sc4ccccc45)cc32)cc1] 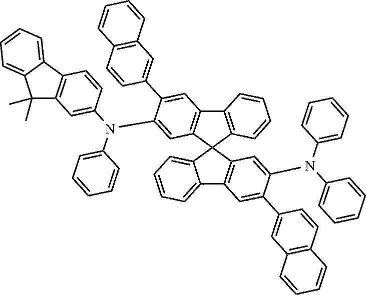 [SMILES: CC1(C)c2ccccc2-c2ccc(N(c3ccccc3)c3cc4c(cc3-c3ccc5ccccc5c3)-c3ccccc3C43c4ccccc4-c4cc(-c5ccc6ccccc6c5)c(N(c5ccccc5)c5ccccc5)cc43)cc21] 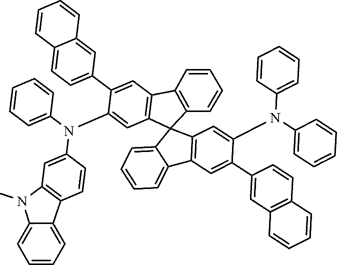 [SMILES: Cn1c2ccccc2c2ccc(N(c3ccccc3)c3cc4c(cc3-c3ccc5ccccc5c3)-c3ccccc3C43c4ccccc4-c4cc(-c5ccc6ccccc6c5)c(N(c5ccccc5)c5ccccc5)cc43)cc21] 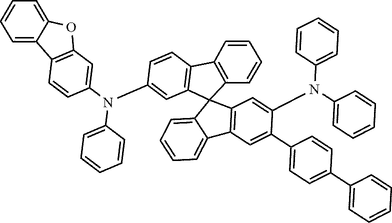 [SMILES: c1ccc(-c2ccc(-c3cc4c(cc3N(c3ccccc3)c3ccccc3)C3(c5ccccc5-c5ccc(N(c6ccccc6)c6ccc7c(c6)oc6ccccc67)cc53)c3ccccc3-4)cc2)cc1] 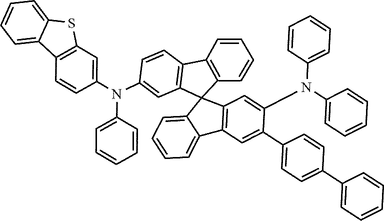 [SMILES: c1ccc(-c2ccc(-c3cc4c(cc3N(c3ccccc3)c3ccccc3)C3(c5ccccc5-c5ccc(N(c6ccccc6)c6ccc7c(c6)sc6ccccc67)cc53)c3ccccc3-4)cc2)cc1] 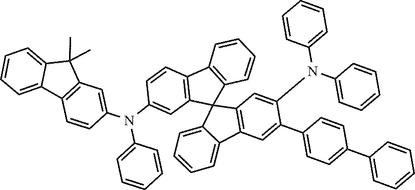 [SMILES: CC1(C)c2ccccc2-c2ccc(N(c3ccccc3)c3ccc4c(c3)C3(c5ccccc5-4)c4ccccc4-c4cc(-c5ccc(-c6ccccc6)cc5)c(N(c5ccccc5)c5ccccc5)cc43)cc21] 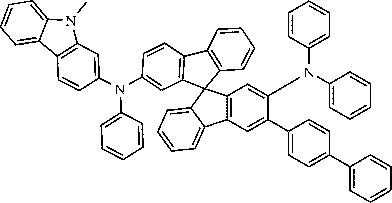 [SMILES: Cn1c2ccccc2c2ccc(N(c3ccccc3)c3ccc4c(c3)C3(c5ccccc5-4)c4ccccc4-c4cc(-c5ccc(-c6ccccc6)cc5)c(N(c5ccccc5)c5ccccc5)cc43)cc21] 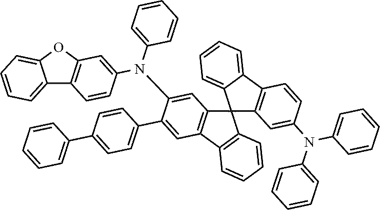 [SMILES: c1ccc(-c2ccc(-c3cc4c(cc3N(c3ccccc3)c3ccc5c(c3)oc3ccccc35)C3(c5ccccc5-c5ccc(N(c6ccccc6)c6ccccc6)cc53)c3ccccc3-4)cc2)cc1] 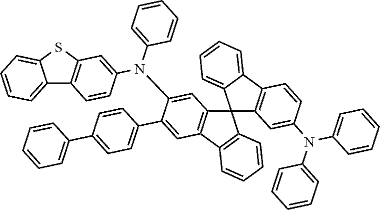 [SMILES: c1ccc(-c2ccc(-c3cc4c(cc3N(c3ccccc3)c3ccc5c(c3)sc3ccccc35)C3(c5ccccc5-c5ccc(N(c6ccccc6)c6ccccc6)cc53)c3ccccc3-4)cc2)cc1] 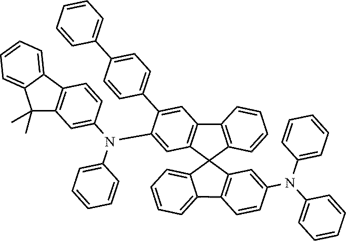 [SMILES: CC1(C)c2ccccc2-c2ccc(N(c3ccccc3)c3cc4c(cc3-c3ccc(-c5ccccc5)cc3)-c3ccccc3C43c4ccccc4-c4ccc(N(c5ccccc5)c5ccccc5)cc43)cc21] 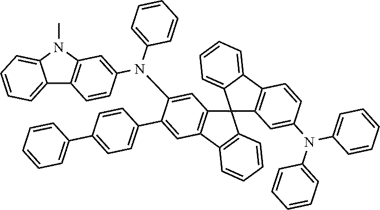 [SMILES: Cn1c2ccccc2c2ccc(N(c3ccccc3)c3cc4c(cc3-c3ccc(-c5ccccc5)cc3)-c3ccccc3C43c4ccccc4-c4ccc(N(c5ccccc5)c5ccccc5)cc43)cc21] 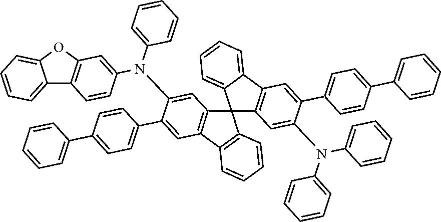 [SMILES: c1ccc(-c2ccc(-c3cc4c(cc3N(c3ccccc3)c3ccccc3)C3(c5ccccc5-4)c4ccccc4-c4cc(-c5ccc(-c6ccccc6)cc5)c(N(c5ccccc5)c5ccc6c(c5)oc5ccccc56)cc43)cc2)cc1] 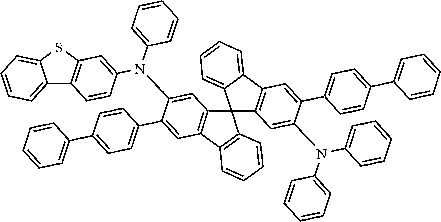 [SMILES: c1ccc(-c2ccc(-c3cc4c(cc3N(c3ccccc3)c3ccccc3)C3(c5ccccc5-4)c4ccccc4-c4cc(-c5ccc(-c6ccccc6)cc5)c(N(c5ccccc5)c5ccc6c(c5)sc5ccccc56)cc43)cc2)cc1] 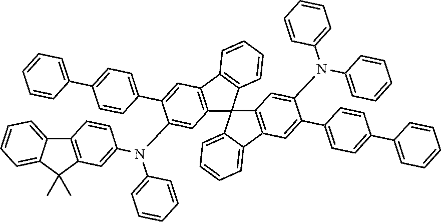 [SMILES: CC1(C)c2ccccc2-c2ccc(N(c3ccccc3)c3cc4c(cc3-c3ccc(-c5ccccc5)cc3)-c3ccccc3C43c4ccccc4-c4cc(-c5ccc(-c6ccccc6)cc5)c(N(c5ccccc5)c5ccccc5)cc43)cc21] 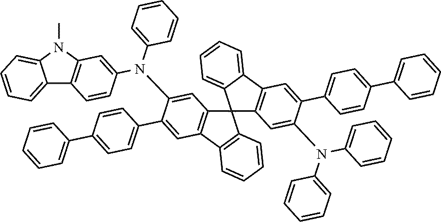 [SMILES: Cn1c2ccccc2c2ccc(N(c3ccccc3)c3cc4c(cc3-c3ccc(-c5ccccc5)cc3)-c3ccccc3C43c4ccccc4-c4cc(-c5ccc(-c6ccccc6)cc5)c(N(c5ccccc5)c5ccccc5)cc43)cc21] 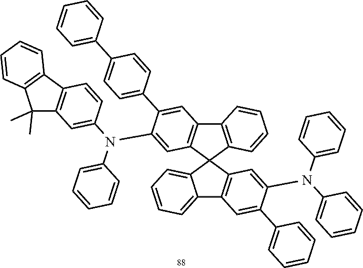 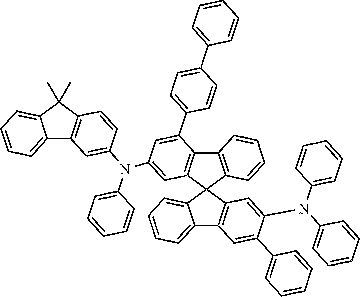 [SMILES: CC1(C)c2ccccc2-c2cc(N(c3ccccc3)c3cc(-c4ccc(-c5ccccc5)cc4)c4c(c3)C3(c5ccccc5-c5cc(-c6ccccc6)c(N(c6ccccc6)c6ccccc6)cc53)c3ccccc3-4)ccc21] 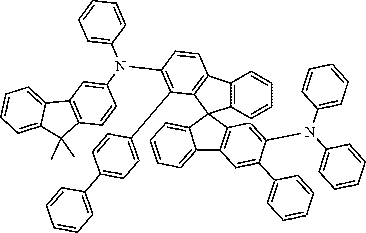 [SMILES: CC1(C)c2ccccc2-c2cc(N(c3ccccc3)c3ccc4c(c3-c3ccc(-c5ccccc5)cc3)C3(c5ccccc5-c5cc(-c6ccccc6)c(N(c6ccccc6)c6ccccc6)cc53)c3ccccc3-4)ccc21] 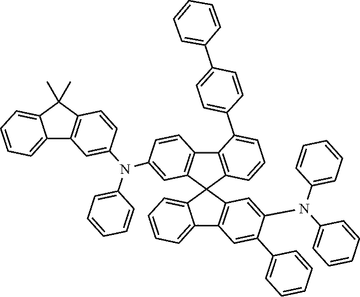 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3cccc(-c5ccc(-c6ccccc6)cc5)c3-4)ccc21] 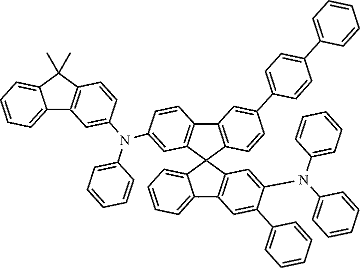 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3ccc(-c5ccc(-c6ccccc6)cc5)cc3-4)ccc21] 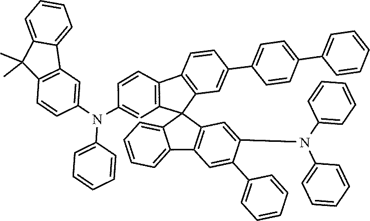 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3cc(-c5ccc(-c6ccccc6)cc5)ccc3-4)ccc21] 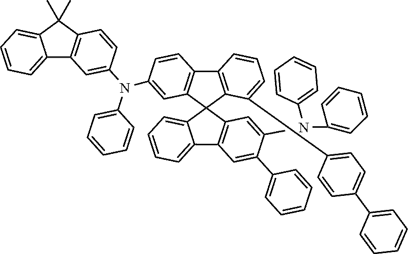 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3c(-c5ccc(-c6ccccc6)cc5)cccc3-4)ccc21] 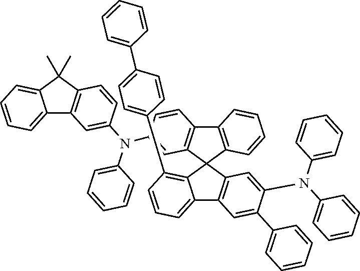 [SMILES: CC1(C)c2ccccc2-c2cc(N(c3ccccc3)c3ccc4c(c3)C3(c5ccccc5-4)c4cc(N(c5ccccc5)c5ccccc5)c(-c5ccccc5)cc4-c4cccc(-c5ccc(-c6ccccc6)cc5)c43)ccc21] 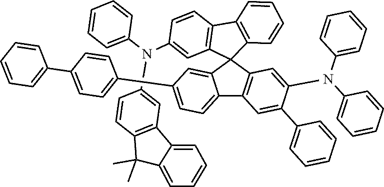 [SMILES: CC1(C)c2ccccc2-c2cc(N(c3ccccc3)c3ccc4c(c3)C3(c5ccccc5-4)c4cc(-c5ccc(-c6ccccc6)cc5)ccc4-c4cc(-c5ccccc5)c(N(c5ccccc5)c5ccccc5)cc43)ccc21] 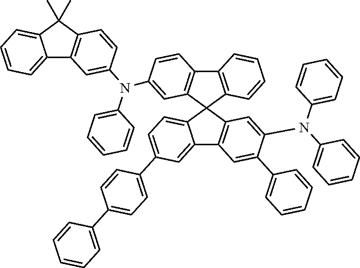 [SMILES: CC1(C)c2ccccc2-c2cc(N(c3ccccc3)c3ccc4c(c3)C3(c5ccccc5-4)c4ccc(-c5ccc(-c6ccccc6)cc5)cc4-c4cc(-c5ccccc5)c(N(c5ccccc5)c5ccccc5)cc43)ccc21] 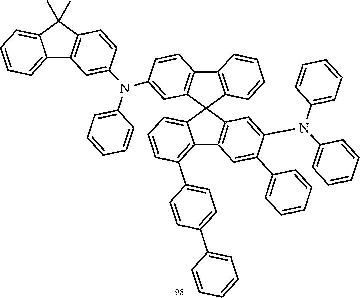 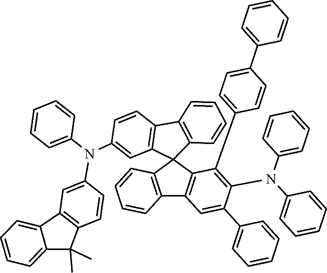 [SMILES: CC1(C)c2ccccc2-c2cc(N(c3ccccc3)c3ccc4c(c3)C3(c5ccccc5-4)c4ccccc4-c4cc(-c5ccccc5)c(N(c5ccccc5)c5ccccc5)c(-c5ccc(-c6ccccc6)cc5)c43)ccc21] 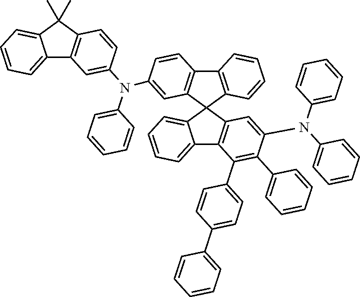 [SMILES: CC1(C)c2ccccc2-c2cc(N(c3ccccc3)c3ccc4c(c3)C3(c5ccccc5-4)c4ccccc4-c4c3cc(N(c3ccccc3)c3ccccc3)c(-c3ccccc3)c4-c3ccc(-c4ccccc4)cc3)ccc21] 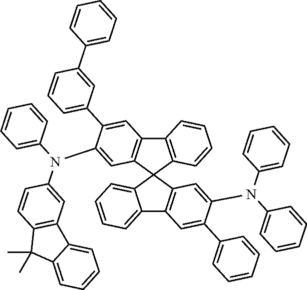 [SMILES: CC1(C)c2ccccc2-c2cc(N(c3ccccc3)c3cc4c(cc3-c3cccc(-c5ccccc5)c3)-c3ccccc3C43c4ccccc4-c4cc(-c5ccccc5)c(N(c5ccccc5)c5ccccc5)cc43)ccc21] 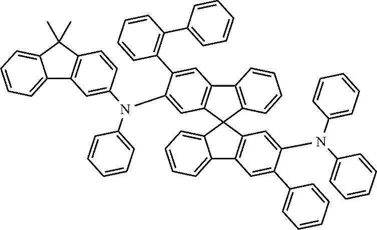 [SMILES: CC1(C)c2ccccc2-c2cc(N(c3ccccc3)c3cc4c(cc3-c3ccccc3-c3ccccc3)-c3ccccc3C43c4ccccc4-c4cc(-c5ccccc5)c(N(c5ccccc5)c5ccccc5)cc43)ccc21] 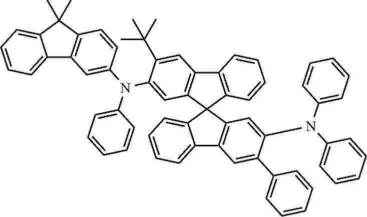 [SMILES: CC(C)(C)c1cc2c(cc1N(c1ccccc1)c1ccc3c(c1)-c1ccccc1C3(C)C)C1(c3ccccc3-c3cc(-c4ccccc4)c(N(c4ccccc4)c4ccccc4)cc31)c1ccccc1-2] 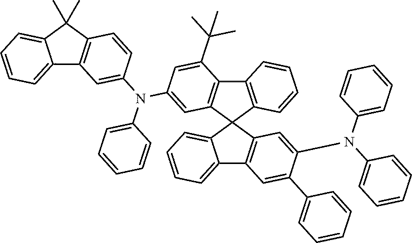 [SMILES: CC(C)(C)c1cc(N(c2ccccc2)c2ccc3c(c2)-c2ccccc2C3(C)C)cc2c1-c1ccccc1C21c2ccccc2-c2cc(-c3ccccc3)c(N(c3ccccc3)c3ccccc3)cc21] 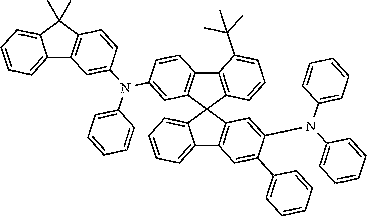 [SMILES: CC(C)(C)c1cccc2c1-c1ccc(N(c3ccccc3)c3ccc4c(c3)-c3ccccc3C4(C)C)cc1C21c2ccccc2-c2cc(-c3ccccc3)c(N(c3ccccc3)c3ccccc3)cc21] 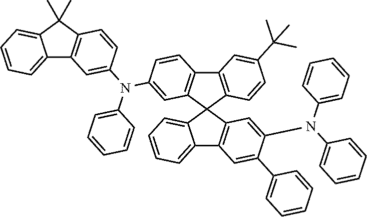 [SMILES: CC(C)(C)c1ccc2c(c1)-c1ccc(N(c3ccccc3)c3ccc4c(c3)-c3ccccc3C4(C)C)cc1C21c2ccccc2-c2cc(-c3ccccc3)c(N(c3ccccc3)c3ccccc3)cc21] 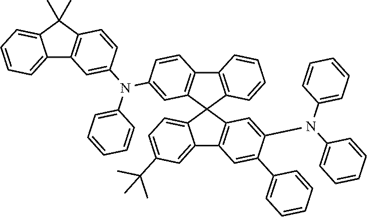 [SMILES: CC(C)(C)c1ccc2c(c1)-c1cc(-c3ccccc3)c(N(c3ccccc3)c3ccccc3)cc1C21c2ccccc2-c2ccc(N(c3ccccc3)c3ccc4c(c3)-c3ccccc3C4(C)C)cc21] 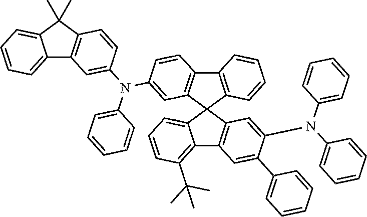 [SMILES: CC(C)(C)c1cccc2c1-c1cc(-c3ccccc3)c(N(c3ccccc3)c3ccccc3)cc1C21c2ccccc2-c2ccc(N(c3ccccc3)c3ccc4c(c3)-c3ccccc3C4(C)C)cc21] 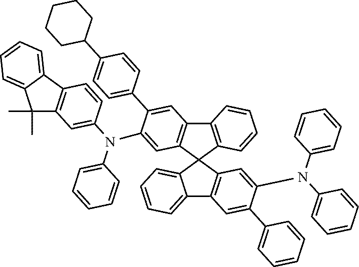 [SMILES: CC1(C)c2ccccc2-c2ccc(N(c3ccccc3)c3cc4c(cc3-c3ccc(C5CCCCC5)cc3)-c3ccccc3C43c4ccccc4-c4cc(-c5ccccc5)c(N(c5ccccc5)c5ccccc5)cc43)cc21] 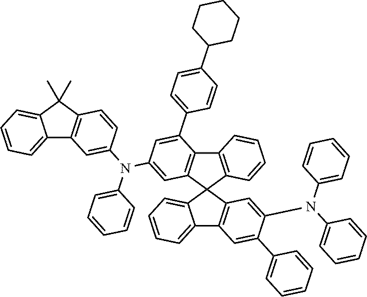 [SMILES: CC1(C)c2ccccc2-c2cc(N(c3ccccc3)c3cc(-c4ccc(C5CCCCC5)cc4)c4c(c3)C3(c5ccccc5-c5cc(-c6ccccc6)c(N(c6ccccc6)c6ccccc6)cc53)c3ccccc3-4)ccc21] 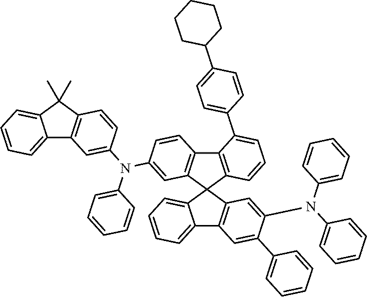 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3cccc(-c5ccc(C6CCCCC6)cc5)c3-4)ccc21] 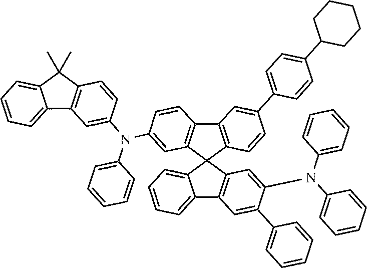 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3ccc(-c5ccc(C6CCCCC6)cc5)cc3-4)ccc21] 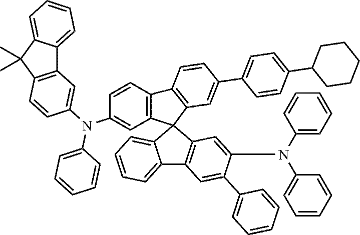 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3cc(-c5ccc(C6CCCCC6)cc5)ccc3-4)ccc21] 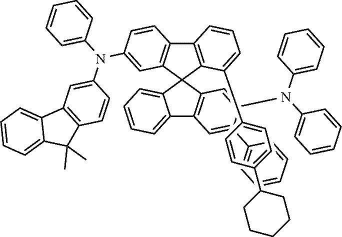 [SMILES: CC1(C)c2ccccc2-c2cc(N(c3ccccc3)c3ccc4c(c3)C3(c5ccccc5-c5cc(-c6ccccc6)c(N(c6ccccc6)c6ccccc6)cc53)c3c(-c5ccc(C6CCCCC6)cc5)cccc3-4)ccc21] 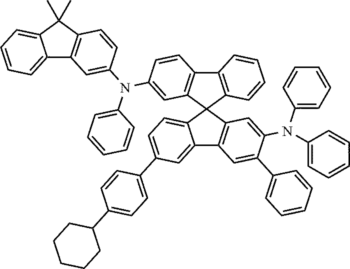 [SMILES: CC1(C)c2ccccc2-c2cc(N(c3ccccc3)c3ccc4c(c3)C3(c5ccccc5-4)c4ccc(-c5ccc(C6CCCCC6)cc5)cc4-c4cc(-c5ccccc5)c(N(c5ccccc5)c5ccccc5)cc43)ccc21] 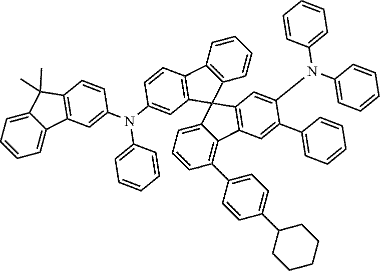 [SMILES: CC1(C)c2ccccc2-c2cc(N(c3ccccc3)c3ccc4c(c3)C3(c5ccccc5-4)c4cc(N(c5ccccc5)c5ccccc5)c(-c5ccccc5)cc4-c4c(-c5ccc(C6CCCCC6)cc5)cccc43)ccc21] 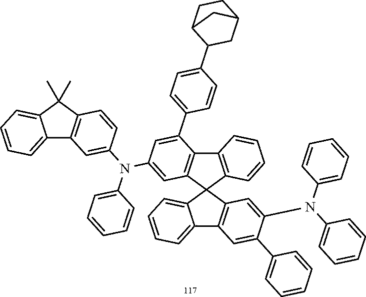 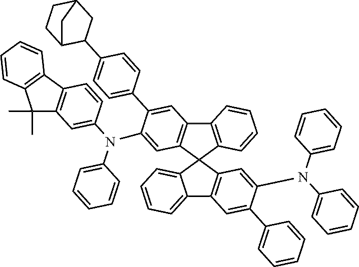 [SMILES: CC1(C)c2ccccc2-c2ccc(N(c3ccccc3)c3cc4c(cc3-c3ccc(C5CC6CCC5C6)cc3)-c3ccccc3C43c4ccccc4-c4cc(-c5ccccc5)c(N(c5ccccc5)c5ccccc5)cc43)cc21] 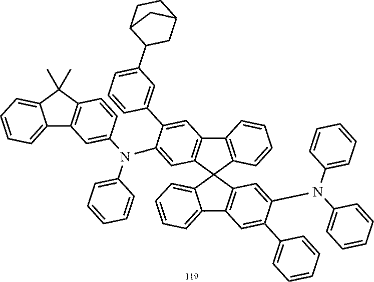 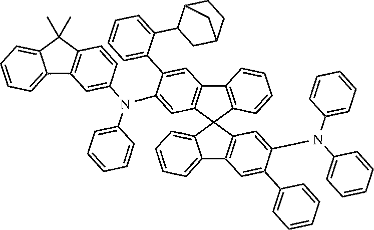 [SMILES: CC1(C)c2ccccc2-c2cc(N(c3ccccc3)c3cc4c(cc3-c3ccccc3C3CC5CCC3C5)-c3ccccc3C43c4ccccc4-c4cc(-c5ccccc5)c(N(c5ccccc5)c5ccccc5)cc43)ccc21] 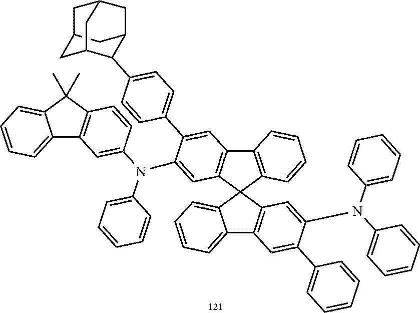 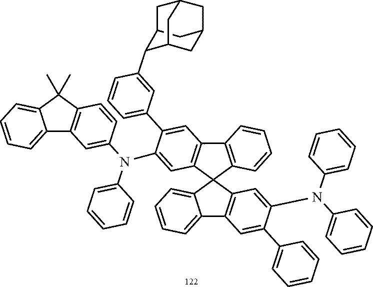 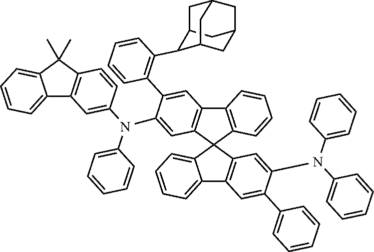 [SMILES: CC1(C)c2ccccc2-c2cc(N(c3ccccc3)c3cc4c(cc3-c3ccccc3C3C5CC6CC(C5)CC3C6)-c3ccccc3C43c4ccccc4-c4cc(-c5ccccc5)c(N(c5ccccc5)c5ccccc5)cc43)ccc21] 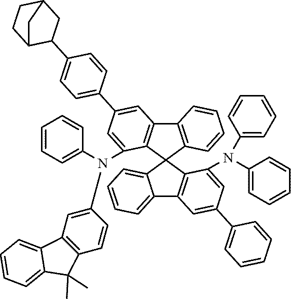 [SMILES: CC1(C)c2ccccc2-c2cc(N(c3ccccc3)c3cc(-c4ccc(C5CC6CCC5C6)cc4)cc4c3C3(c5ccccc5-c5cc(-c6ccccc6)cc(N(c6ccccc6)c6ccccc6)c53)c3ccccc3-4)ccc21] 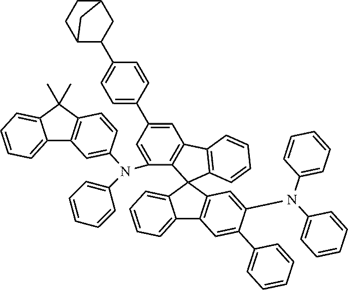 [SMILES: CC1(C)c2ccccc2-c2cc(N(c3ccccc3)c3cc(-c4ccc(C5CC6CCC5C6)cc4)cc4c3C3(c5ccccc5-c5cc(-c6ccccc6)c(N(c6ccccc6)c6ccccc6)cc53)c3ccccc3-4)ccc21] 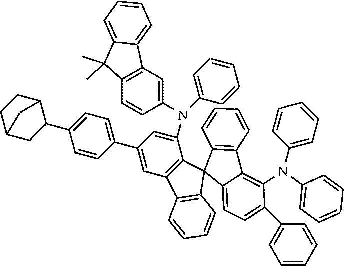 [SMILES: CC1(C)c2ccccc2-c2cc(N(c3ccccc3)c3cc(-c4ccc(C5CC6CCC5C6)cc4)cc4c3C3(c5ccccc5-4)c4ccccc4-c4c3ccc(-c3ccccc3)c4N(c3ccccc3)c3ccccc3)ccc21] 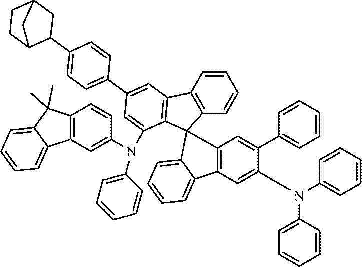 [SMILES: CC1(C)c2ccccc2-c2cc(N(c3ccccc3)c3cc(-c4ccc(C5CC6CCC5C6)cc4)cc4c3C3(c5ccccc5-c5cc(N(c6ccccc6)c6ccccc6)c(-c6ccccc6)cc53)c3ccccc3-4)ccc21] 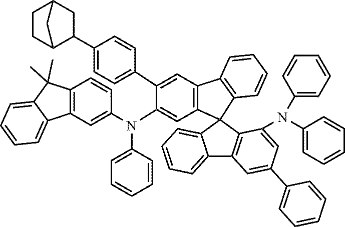 [SMILES: CC1(C)c2ccccc2-c2cc(N(c3ccccc3)c3cc4c(cc3-c3ccc(C5CC6CCC5C6)cc3)-c3ccccc3C43c4ccccc4-c4cc(-c5ccccc5)cc(N(c5ccccc5)c5ccccc5)c43)ccc21] 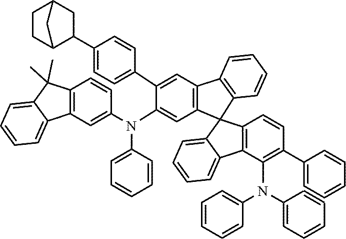 [SMILES: CC1(C)c2ccccc2-c2cc(N(c3ccccc3)c3cc4c(cc3-c3ccc(C5CC6CCC5C6)cc3)-c3ccccc3C43c4ccccc4-c4c3ccc(-c3ccccc3)c4N(c3ccccc3)c3ccccc3)ccc21] 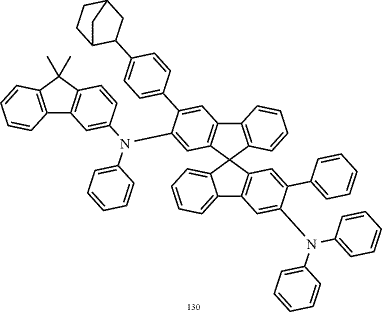 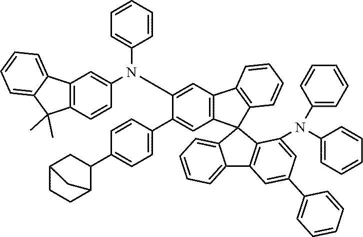 [SMILES: CC1(C)c2ccccc2-c2cc(N(c3ccccc3)c3cc4c(cc3-c3ccc(C5CC6CCC5C6)cc3)C3(c5ccccc5-4)c4ccccc4-c4cc(-c5ccccc5)cc(N(c5ccccc5)c5ccccc5)c43)ccc21] 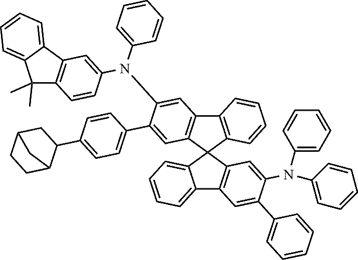 [SMILES: CC1(C)c2ccccc2-c2cc(N(c3ccccc3)c3cc4c(cc3-c3ccc(C5CC6CCC5C6)cc3)C3(c5ccccc5-4)c4ccccc4-c4cc(-c5ccccc5)c(N(c5ccccc5)c5ccccc5)cc43)ccc21] 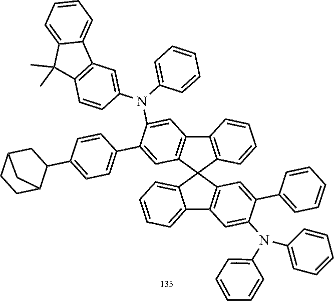 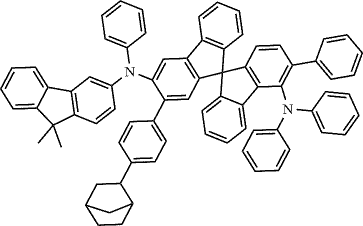 [SMILES: CC1(C)c2ccccc2-c2cc(N(c3ccccc3)c3cc4c(cc3-c3ccc(C5CC6CCC5C6)cc3)C3(c5ccccc5-4)c4ccccc4-c4c3ccc(-c3ccccc3)c4N(c3ccccc3)c3ccccc3)ccc21] 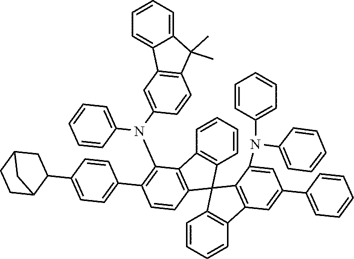 [SMILES: CC1(C)c2ccccc2-c2cc(N(c3ccccc3)c3c(-c4ccc(C5CC6CCC5C6)cc4)ccc4c3-c3ccccc3C43c4ccccc4-c4cc(-c5ccccc5)cc(N(c5ccccc5)c5ccccc5)c43)ccc21] 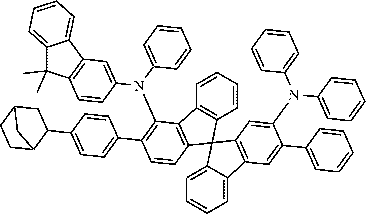 [SMILES: CC1(C)c2ccccc2-c2cc(N(c3ccccc3)c3c(-c4ccc(C5CC6CCC5C6)cc4)ccc4c3-c3ccccc3C43c4ccccc4-c4cc(-c5ccccc5)c(N(c5ccccc5)c5ccccc5)cc43)ccc21] 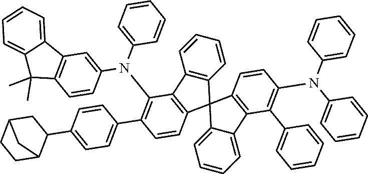 [SMILES: CC1(C)c2ccccc2-c2cc(N(c3ccccc3)c3c(-c4ccc(C5CC6CCC5C6)cc4)ccc4c3-c3ccccc3C43c4ccccc4-c4c3ccc(N(c3ccccc3)c3ccccc3)c4-c3ccccc3)ccc21] 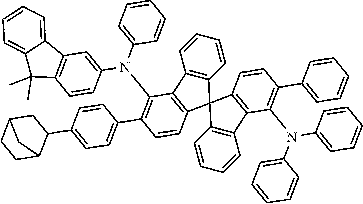 [SMILES: CC1(C)c2ccccc2-c2cc(N(c3ccccc3)c3c(-c4ccc(C5CC6CCC5C6)cc4)ccc4c3-c3ccccc3C43c4ccccc4-c4c3ccc(-c3ccccc3)c4N(c3ccccc3)c3ccccc3)ccc21] 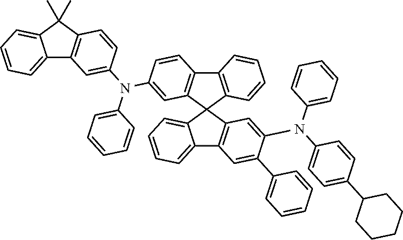 [SMILES: CC1(C)c2ccccc2-c2cc(N(c3ccccc3)c3ccc4c(c3)C3(c5ccccc5-4)c4ccccc4-c4cc(-c5ccccc5)c(N(c5ccccc5)c5ccc(C6CCCCC6)cc5)cc43)ccc21] 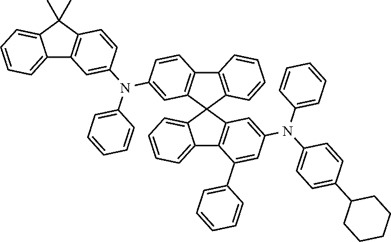 [SMILES: CC1(C)c2ccccc2-c2cc(N(c3ccccc3)c3ccc4c(c3)C3(c5ccccc5-4)c4ccccc4-c4c(-c5ccccc5)cc(N(c5ccccc5)c5ccc(C6CCCCC6)cc5)cc43)ccc21] 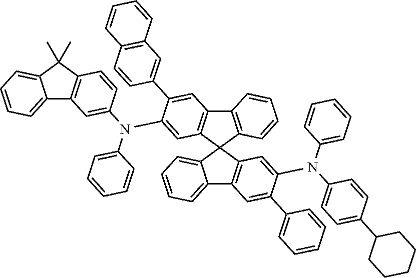 [SMILES: CC1(C)c2ccccc2-c2cc(N(c3ccccc3)c3cc4c(cc3-c3ccc5ccccc5c3)-c3ccccc3C43c4ccccc4-c4cc(-c5ccccc5)c(N(c5ccccc5)c5ccc(C6CCCCC6)cc5)cc43)ccc21] 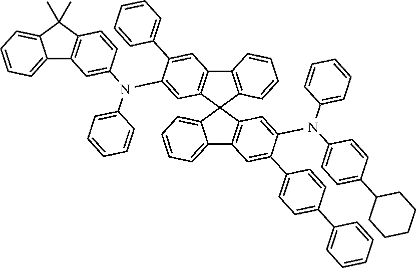 [SMILES: CC1(C)c2ccccc2-c2cc(N(c3ccccc3)c3cc4c(cc3-c3ccccc3)-c3ccccc3C43c4ccccc4-c4cc(-c5ccc(-c6ccccc6)cc5)c(N(c5ccccc5)c5ccc(C6CCCCC6)cc5)cc43)ccc21] 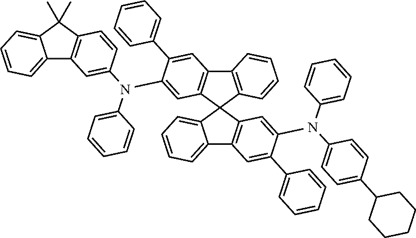 [SMILES: CC1(C)c2ccccc2-c2cc(N(c3ccccc3)c3cc4c(cc3-c3ccccc3)-c3ccccc3C43c4ccccc4-c4cc(-c5ccccc5)c(N(c5ccccc5)c5ccc(C6CCCCC6)cc5)cc43)ccc21] 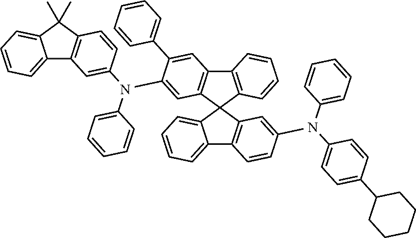 [SMILES: CC1(C)c2ccccc2-c2cc(N(c3ccccc3)c3cc4c(cc3-c3ccccc3)-c3ccccc3C43c4ccccc4-c4ccc(N(c5ccccc5)c5ccc(C6CCCCC6)cc5)cc43)ccc21] 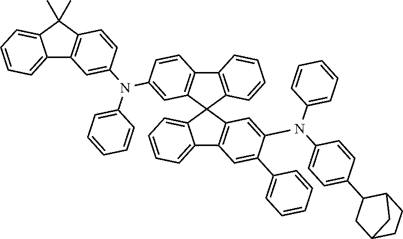 [SMILES: CC1(C)c2ccccc2-c2cc(N(c3ccccc3)c3ccc4c(c3)C3(c5ccccc5-4)c4ccccc4-c4cc(-c5ccccc5)c(N(c5ccccc5)c5ccc(C6CC7CCC6C7)cc5)cc43)ccc21] 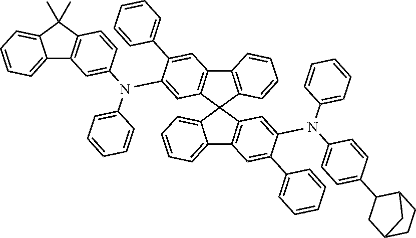 [SMILES: CC1(C)c2ccccc2-c2cc(N(c3ccccc3)c3cc4c(cc3-c3ccccc3)-c3ccccc3C43c4ccccc4-c4cc(-c5ccccc5)c(N(c5ccccc5)c5ccc(C6CC7CCC6C7)cc5)cc43)ccc21] 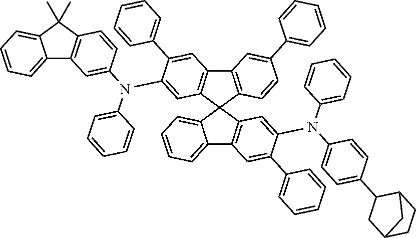 [SMILES: CC1(C)c2ccccc2-c2cc(N(c3ccccc3)c3cc4c(cc3-c3ccccc3)-c3cc(-c5ccccc5)ccc3C43c4ccccc4-c4cc(-c5ccccc5)c(N(c5ccccc5)c5ccc(C6CC7CCC6C7)cc5)cc43)ccc21] 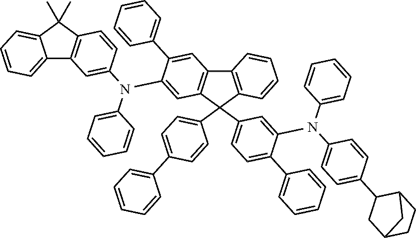 [SMILES: CC1(C)c2ccccc2-c2cc(N(c3ccccc3)c3cc4c(cc3-c3ccccc3)-c3ccccc3C4(c3ccc(-c4ccccc4)cc3)c3ccc(-c4ccccc4)c(N(c4ccccc4)c4ccc(C5CC6CCC5C6)cc4)c3)ccc21] 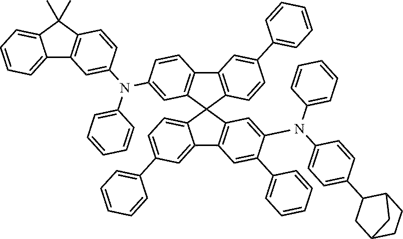 [SMILES: CC1(C)c2ccccc2-c2cc(N(c3ccccc3)c3ccc4c(c3)C3(c5ccc(-c6ccccc6)cc5-4)c4ccc(-c5ccccc5)cc4-c4cc(-c5ccccc5)c(N(c5ccccc5)c5ccc(C6CC7CCC6C7)cc5)cc43)ccc21] 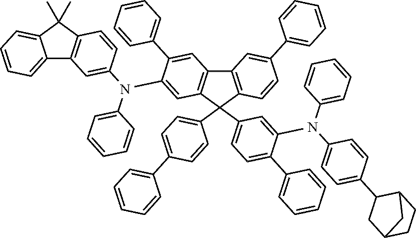 [SMILES: CC1(C)c2ccccc2-c2cc(N(c3ccccc3)c3cc4c(cc3-c3ccccc3)-c3cc(-c5ccccc5)ccc3C4(c3ccc(-c4ccccc4)cc3)c3ccc(-c4ccccc4)c(N(c4ccccc4)c4ccc(C5CC6CCC5C6)cc4)c3)ccc21] 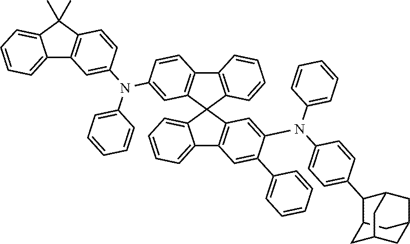 [SMILES: CC1(C)c2ccccc2-c2cc(N(c3ccccc3)c3ccc4c(c3)C3(c5ccccc5-4)c4ccccc4-c4cc(-c5ccccc5)c(N(c5ccccc5)c5ccc(C6C7CC8CC(C7)CC6C8)cc5)cc43)ccc21] 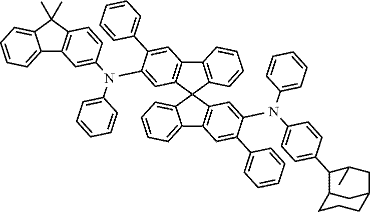 [SMILES: CC1CC2CCCC(C2)C1c1ccc(N(c2ccccc2)c2cc3c(cc2-c2ccccc2)-c2ccccc2C32c3ccccc3-c3cc(-c4ccccc4)c(N(c4ccccc4)c4ccc5c(c4)-c4ccccc4C5(C)C)cc32)cc1] 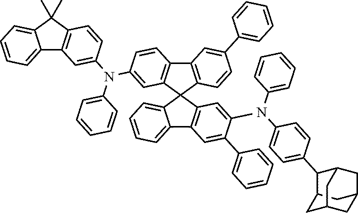 [SMILES: CC1(C)c2ccccc2-c2cc(N(c3ccccc3)c3ccc4c(c3)C3(c5ccccc5-c5cc(-c6ccccc6)c(N(c6ccccc6)c6ccc(C7C8CC9CC(C8)CC7C9)cc6)cc53)c3ccc(-c5ccccc5)cc3-4)ccc21] 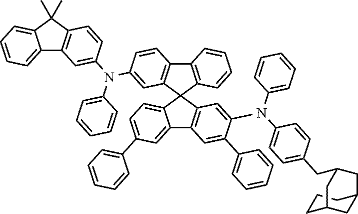 [SMILES: CC1(C)c2ccccc2-c2cc(N(c3ccccc3)c3ccc4c(c3)C3(c5ccccc5-4)c4ccc(-c5ccccc5)cc4-c4cc(-c5ccccc5)c(N(c5ccccc5)c5ccc(CC6CC7CCCC(C7)C6)cc5)cc43)ccc21] 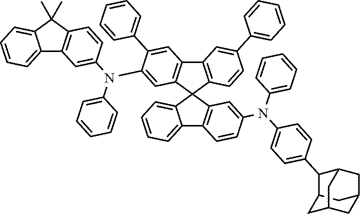 [SMILES: CC1(C)c2ccccc2-c2cc(N(c3ccccc3)c3cc4c(cc3-c3ccccc3)-c3cc(-c5ccccc5)ccc3C43c4ccccc4-c4ccc(N(c5ccccc5)c5ccc(C6C7CC8CC(C7)CC6C8)cc5)cc43)ccc21] 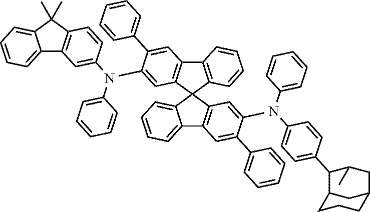 [SMILES: CC1CC2CCCC(C2)C1c1ccc(N(c2ccccc2)c2cc3c(cc2-c2ccccc2)-c2ccccc2C32c3ccccc3-c3cc(-c4ccccc4)c(N(c4ccccc4)c4ccc5c(c4)-c4ccccc4C5(C)C)cc32)cc1] 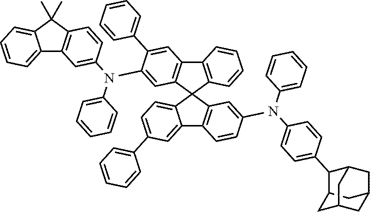 [SMILES: CC1(C)c2ccccc2-c2cc(N(c3ccccc3)c3cc4c(cc3-c3ccccc3)-c3ccccc3C43c4ccc(-c5ccccc5)cc4-c4ccc(N(c5ccccc5)c5ccc(C6C7CC8CC(C7)CC6C8)cc5)cc43)ccc21] 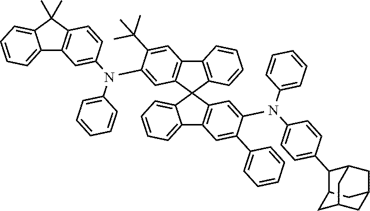 [SMILES: CC(C)(C)c1cc2c(cc1N(c1ccccc1)c1ccc3c(c1)-c1ccccc1C3(C)C)C1(c3ccccc3-c3cc(-c4ccccc4)c(N(c4ccccc4)c4ccc(C5C6CC7CC(C6)CC5C7)cc4)cc31)c1ccccc1-2] 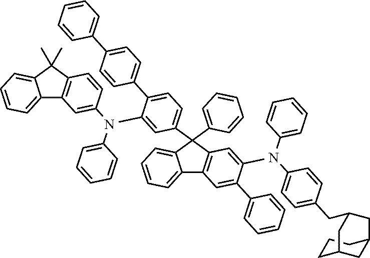 [SMILES: CC1(C)c2ccccc2-c2cc(N(c3ccccc3)c3cc(C4(c5ccccc5)c5ccccc5-c5cc(-c6ccccc6)c(N(c6ccccc6)c6ccc(CC7CC8CCCC(C8)C7)cc6)cc54)ccc3-c3ccc(-c4ccccc4)cc3)ccc21] 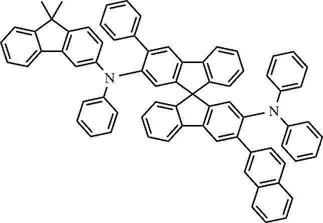 [SMILES: CC1(C)c2ccccc2-c2cc(N(c3ccccc3)c3cc4c(cc3-c3ccccc3)-c3ccccc3C43c4ccccc4-c4cc(-c5ccc6ccccc6c5)c(N(c5ccccc5)c5ccccc5)cc43)ccc21] 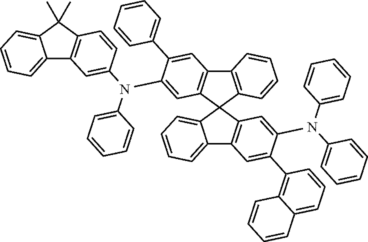 [SMILES: CC1(C)c2ccccc2-c2cc(N(c3ccccc3)c3cc4c(cc3-c3ccccc3)-c3ccccc3C43c4ccccc4-c4cc(-c5cccc6ccccc56)c(N(c5ccccc5)c5ccccc5)cc43)ccc21] 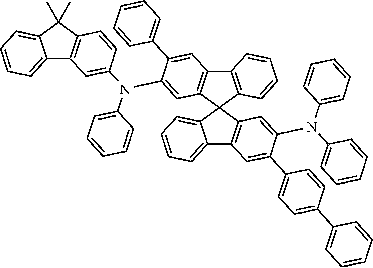 [SMILES: CC1(C)c2ccccc2-c2cc(N(c3ccccc3)c3cc4c(cc3-c3ccccc3)-c3ccccc3C43c4ccccc4-c4cc(-c5ccc(-c6ccccc6)cc5)c(N(c5ccccc5)c5ccccc5)cc43)ccc21] 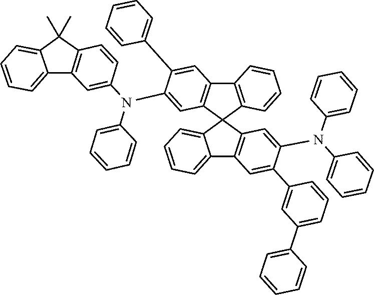 [SMILES: CC1(C)c2ccccc2-c2cc(N(c3ccccc3)c3cc4c(cc3-c3ccccc3)-c3ccccc3C43c4ccccc4-c4cc(-c5cccc(-c6ccccc6)c5)c(N(c5ccccc5)c5ccccc5)cc43)ccc21] 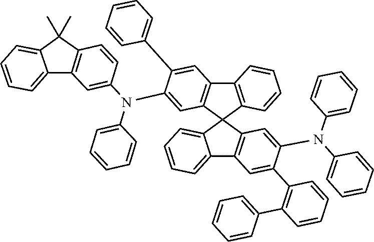 [SMILES: CC1(C)c2ccccc2-c2cc(N(c3ccccc3)c3cc4c(cc3-c3ccccc3)-c3ccccc3C43c4ccccc4-c4cc(-c5ccccc5-c5ccccc5)c(N(c5ccccc5)c5ccccc5)cc43)ccc21] 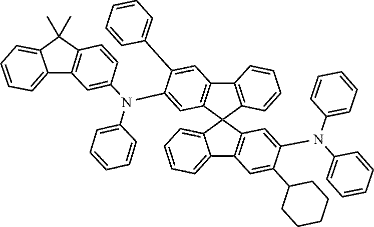 [SMILES: CC1(C)c2ccccc2-c2cc(N(c3ccccc3)c3cc4c(cc3-c3ccccc3)-c3ccccc3C43c4ccccc4-c4cc(C5CCCCC5)c(N(c5ccccc5)c5ccccc5)cc43)ccc21] 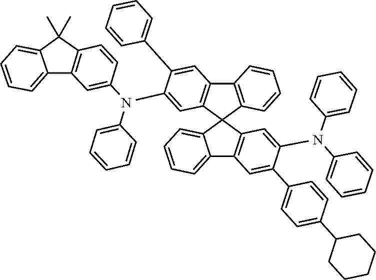 [SMILES: CC1(C)c2ccccc2-c2cc(N(c3ccccc3)c3cc4c(cc3-c3ccccc3)-c3ccccc3C43c4ccccc4-c4cc(-c5ccc(C6CCCCC6)cc5)c(N(c5ccccc5)c5ccccc5)cc43)ccc21] 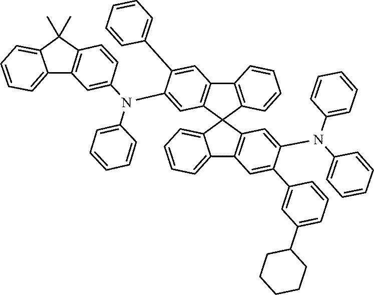 [SMILES: CC1(C)c2ccccc2-c2cc(N(c3ccccc3)c3cc4c(cc3-c3ccccc3)-c3ccccc3C43c4ccccc4-c4cc(-c5cccc(C6CCCCC6)c5)c(N(c5ccccc5)c5ccccc5)cc43)ccc21] 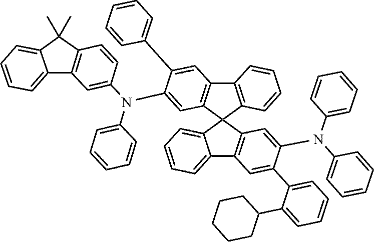 [SMILES: CC1(C)c2ccccc2-c2cc(N(c3ccccc3)c3cc4c(cc3-c3ccccc3)-c3ccccc3C43c4ccccc4-c4cc(-c5ccccc5C5CCCCC5)c(N(c5ccccc5)c5ccccc5)cc43)ccc21] 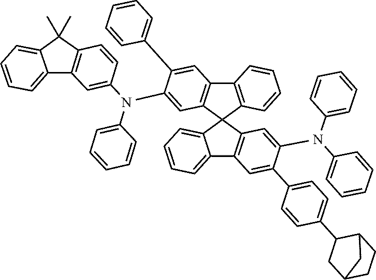 [SMILES: CC1(C)c2ccccc2-c2cc(N(c3ccccc3)c3cc4c(cc3-c3ccccc3)-c3ccccc3C43c4ccccc4-c4cc(-c5ccc(C6CC7CCC6C7)cc5)c(N(c5ccccc5)c5ccccc5)cc43)ccc21] 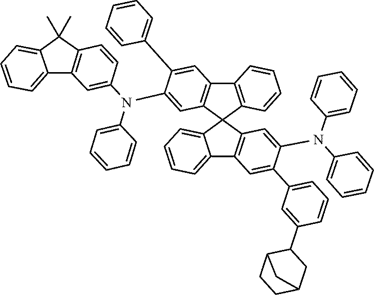 [SMILES: CC1(C)c2ccccc2-c2cc(N(c3ccccc3)c3cc4c(cc3-c3ccccc3)-c3ccccc3C43c4ccccc4-c4cc(-c5cccc(C6CC7CCC6C7)c5)c(N(c5ccccc5)c5ccccc5)cc43)ccc21] 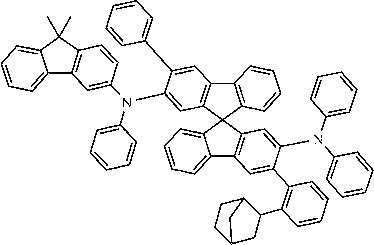 [SMILES: CC1(C)c2ccccc2-c2cc(N(c3ccccc3)c3cc4c(cc3-c3ccccc3)-c3ccccc3C43c4ccccc4-c4cc(-c5ccccc5C5CC6CCC5C6)c(N(c5ccccc5)c5ccccc5)cc43)ccc21] 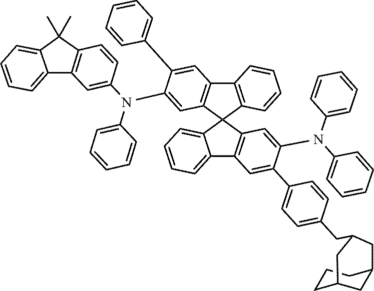 [SMILES: CC1(C)c2ccccc2-c2cc(N(c3ccccc3)c3cc4c(cc3-c3ccccc3)-c3ccccc3C43c4ccccc4-c4cc(-c5ccc(CC6CC7CCCC(C7)C6)cc5)c(N(c5ccccc5)c5ccccc5)cc43)ccc21] 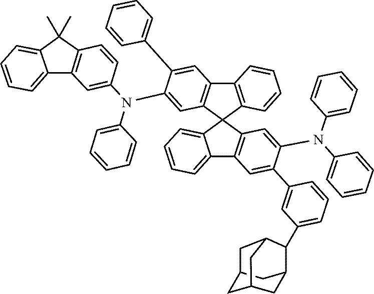 [SMILES: CC1(C)c2ccccc2-c2cc(N(c3ccccc3)c3cc4c(cc3-c3ccccc3)-c3ccccc3C43c4ccccc4-c4cc(-c5cccc(C6C7CC8CC(C7)CC6C8)c5)c(N(c5ccccc5)c5ccccc5)cc43)ccc21] 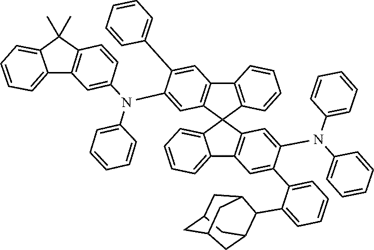 [SMILES: CC1(C)c2ccccc2-c2cc(N(c3ccccc3)c3cc4c(cc3-c3ccccc3)-c3ccccc3C43c4ccccc4-c4cc(-c5ccccc5C5C6CC7CC(C6)CC5C7)c(N(c5ccccc5)c5ccccc5)cc43)ccc21] 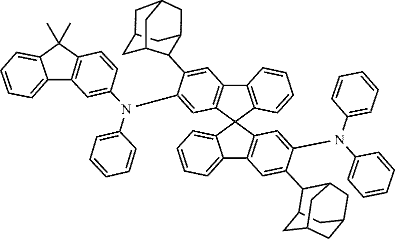 [SMILES: CC1(C)c2ccccc2-c2cc(N(c3ccccc3)c3cc4c(cc3C3C5CC6CC(C5)CC3C6)-c3ccccc3C43c4ccccc4-c4cc(C5C6CC7CC(C6)CC5C7)c(N(c5ccccc5)c5ccccc5)cc43)ccc21]
.

13. The light-emitting device of claim 1, wherein the layer is a hole transport layer.

14. The light-emitting device of claim 1, wherein the emission layer emits blue light.

15. The light-emitting device of claim 1, further comprising a capping layer.

16. The light-emitting device of claim 15, wherein the capping layer is outside the first electrode and/or outside the second electrode.

17. An electronic apparatus comprising the light-emitting device of claim 1.

18. The electronic apparatus of claim 17, further comprising a thin-film transistor,
wherein the thin-film transistor comprises a source electrode and a drain electrode, and
the first electrode of the light-emitting device is electrically connected to at least one selected from the source electrode and the drain electrode of the thin-film transistor.

19. The electronic apparatus of claim 17, further comprising a color filter, a color conversion layer, a touch screen layer, a polarizing layer, or any combination thereof.

20. A compound represented by Formula 1: 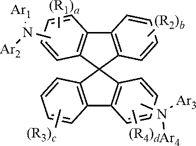
wherein, in Formula 1,
Ar₁ to Ar₄ and R₁ to R₄ are each independently selected from hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₈-C₆₀ monovalent non-aromatic condensed polycyclic group unsubstituted or substituted with at least one R₁₀ₐ, a C₁-C₆₀ monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), and —P(═S)(Q₁)(Q₂),
a and d are each independently an integer from 1 to 3,
b and c are each independently an integer from 1 to 4,
the sum of a and d is an integer of 1 or more, and a case in which each of R₁ and R₄ is hydrogen or deuterium is excluded,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.
